(19) United States
(12) Patent Application Publication
(10) Pub. No.: US 2006/0172019 A1
(43) Pub. Date: Aug. 3, 2006

(54) Cannabinoid receptor inverse agonists and neutral antagonists as therapeutic agents for the treatment of bone disorders

(76) Inventors: Stuart Hamilton Ralston, Aberdeen (GB); Ian Robert Greig, Aberdeen (GB); Ruth Alexandra Ross, Aberdeen (GB); Aymen Ibrahim Idris Mohamed, Aberdeen (GB); Robert Jurgen Van't Hof, Aberdeen (GB)

Correspondence Address: NIXON & VANDERHYE, PC, 901 NORTH GLEBE ROAD, 11TH FLOOR, ARLINGTON, VA, 22203, US

(21) Appl. No.: 10/548,198

(22) PCT Filed: Mar. 2, 2004
(86) PCT No.: PCT/GB04/00858

§ 371 Date: Sep. 7, 2005

(30) Foreign Application Priority Data
Mar. 7, 2003 (GB) 03053436
Jul. 23, 2003 (GB) 03172418
Oct. 16, 2003 (GB) 03242831

(51) Int. Cl. A61K 36/185; A61K 31/5377; A61K 31/4709; A61K 31/454; A61K 31/415

(52) U.S. Cl. 424/725; 514/326; 514/406; 514/232.5; 514/312

(57) ABSTRACT

The present invention pertains to cannabinoid (CB) receptor inverse agonists and neutral antagonists, and especially CB1 and CB2 inverse agonists and neutral antagonists; such as, for example, certain pyrazole compounds; their use in the inhibition of osteoclasts (for example, the inhibition of the survival, formation, and/or activity of osteoclasts), and/or in the inhibition of bone resorption; their use in connection with treatment of bone disorders, such as conditions mediated by osteoclasts (e.g., increased osteoclast activity) and/or characterised by (e.g., increased) bone resorption, such as osteoporosis (e.g., osteoporosis not associated with inflammation; e.g., osteoporosis associated with a genetic predisposition, sex hormone deficiency, or ageing), cancer associated bone disease, and Paget's disease of bone.

### RELATED APPLICATIONS

This application is related to: United Kingdom patent application GB 0305343.6 filed 7 Mar. 2003; United Kingdom patent application GB 0317241.8 filed 23 Jul. 2003; and United Kingdom patent application GB 0324283.1 filed 16 Oct. 2003; the contents of each of which are incorporated herein by reference in their entirety.

### TECHNICAL FIELD

The present invention pertains to cannabinoid (CB) receptor inverse agonists and neutral antagonists, and especially CB1 and CB2 inverse agonists and neutral antagonists; such as, for example, certain pyrazole compounds; their use in the inhibition of osteoclasts (for example, the inhibition of the survival, formation, and/or activity of osteoclasts), and/or in the inhibition of bone resorption; their use in connection with treatment of bone disorders, such as conditions mediated by osteoclasts (e.g., increased osteoclast activity) and/or characterised by (e.g., increased) bone resorption, such as osteoporosis (e.g., osteoporosis not associated with inflammation; e.g., osteoporosis associated with a genetic predisposition, sex hormone deficiency, or ageing), cancer associated bone disease, and Paget's disease of bone.

### BACKGROUND

Throughout this specification, including any claims which follow, unless the context requires otherwise, the word “comprise,” and variations such as “comprises” and “comprising,” will be understood to imply the inclusion of a stated integer or step or group of integers or steps, but not the exclusion of any other integer or step or group of integers or steps.

It must be noted that, as used in the specification and any appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Functions of Bone

The function of bone is to provide mechanical support for joints, tendons and ligaments, to protect vital organs from damage and to act as a reservoir for calcium and phosphate in the preservation of normal mineral homeostasis. Diseases of bone compromise these functions, leading to clinical problems such as bone pain, bone deformity, fracture and abnormalities of calcium and phosphate homeostasis.

Types of Bone

The normal skeleton contains two types of bone; cortical or compact bone, which makes up most of shafts (diaphysis) of the long bones such as the femur and tibia, and trabecular or spongy bone which makes up most of the vertebral bodies and the ends of the long bones.

Trabecular bone has a greater surface area than cortical bone and because of this is remodeled more rapidly. This means that conditions associated with increased bone turnover tend to affect trabecular bone more quickly and more profoundly than cortical bone. Cortical bone is arranged in so-called Haversian systems which consists of a series of concentric lamellae of collagen fibres surrounding a central canal that contains blood vessels. Nutrients reach the central parts of the bone by an interconnecting system of canaliculi that run between osteocytes buried deep within bone matrix and lining cells on the bone surface. Trabecular bone has a similar structure, but here the lamellae run in parallel to the bone surface, rather than concentrically as in cortical bone.

Bone Composition

The organic component of bone matrix comprises mainly of type I collagen; a fibrillar protein formed from three protein chains, wound together in a triple helix. Collagen type I is laid down by bone forming cells (osteoblasts) in organised parallel sheets (lamellae) and subsequently the collagen chains become cross-linked by specialised covalent bonds which help to give bone its tensile strength. When bone is formed rapidly (for example in Paget's disease, or in bone metastases), the lamellae are laid down in a disorderly fashion giving rise to “woven bone”, which is mechanically weak and easily fractured. Bone matrix also contains small amounts of other collagens and several non-collagenous proteins and glycoproteins. Some of these, such as osteocalcin, are specific to bone, whereas others, such as osteopontin and fibronectin and various peptide growth factors are also found in other connective tissues. The function of non-collagenous bone proteins is unclear, but it is thought that they are involved in mediating the attachment of bone cells to bone matrix, and in regulating bone cell activity during the process of bone remodelling. The organic component of bone forms a framework upon which mineralisation occurs. During bone formation, osteoblasts lay down uncalcified bone matrix (osteoid) which contains the components described above and small amounts of other proteins, which are adsorbed from extracellular fluid. After a lag phase of about 10 days, the matrix becomes mineralised, as hydroxyapatite ((Ca₁₀(PO₄)₆(OH)₂) crystals are deposited in the spaces between collagen fibrils. Mineralisation confers upon bone the property of mechanical rigidity, which complements the tensile strength, and elasticity derived from bone collagen.

Bone Cell Function and Bone Remodelling

The mechanical integrity of the skeleton is maintained by the process of bone remodelling, which occurs throughout life, in order that damaged bone can be replaced by new bone. Remodelling can be divided into four phases; resorption; reversal, formation and quiescence (see, e.g., Raisz, 1988; Mundy, 1996). At any one time approximately 10% of bone surface in the adult skeleton is undergoing active remodeled whereas the remaining 90% is quiescent.

Osteoclast Formation and Differentiation

Remodelling commences with attraction of bone resorbing cells (osteoclasts) to the site, which is to be resorbed. These are multinucleated phagocytic cells, rich in the enzyme tartrate-resistant acid phosphatase, which are formed by fusion of precursors derived from the cells of monocyte/macrophage lineage. Recent work has identified several molecules that are of key importance in the regulation of osteoclast differentiation (see, e.g., Ralston, 1997). The transcription factor PU-1 which is expressed in early osteoclast precursors is necessary for the initial stages of osteoclast and monocyte differentiation, whereas other transcription factors including c-fos and NFkB play an essential role in stimulating differentiation of committed precursors to mature osteoclasts. Osteoclast formation and activation is also dependent on close contact between osteoclast precursors and bone marrow stromal cells. Stromal cells secrete the cytokine M-CSF (macrophage colony stimulating factor), which is essential for differentiation of both osteoclasts and macrophages from a common precursor. Stromal cells also express a molecule called RANK ligand (RANKL) on the cell surface, which interacts with another cell surface receptor present on osteoclast precursors called RANK (Receptor Activator of Nuclear Factor Kappa B) to promote differentiation of osteoclast precursors to mature osteoclasts. The RANK-RANKL interaction is blocked by another molecule called Osteoprotegerin (OPG), which is a “decoy” ligand for RANK and which acts a potent inhibitor of osteoclast formation (see, e.g., Kong et al., 1999; Yasuda et al., 1998). Recent work suggests that many of the factors that promote osteoclast formation and bone resorption do so by regulating expression of these molecules.

Mature osteoclasts form a tight seal over the bone surface and resorb bone by secreting hydrochloric acid and proteolytic enzymes through the “ruffled border” into a space beneath the osteoclast (Howship's lacuna). Formation of this ruffled border is critically dependent on the presence of c-src, a cell membrane associated signalling protein. The hydrochloric acid secreted by osteoclasts dissolves hydroxyapatite and allows proteolytic enzymes (mainly Cathepsin K and matrix metalloproteinases) to degrade collagen and other matrix proteins. Molecules which have been identified as being important in regulating osteoclast activity include; carbonic anhydrase II (Ca-II) which catalyses formation of hydrogen ions within osteoclasts; TCIRG1, which encodes a subunit of the osteoclast proton pump, and Cathepsin K which degrades collagen and other non-collagenous proteins. Deficiency of these proteins causes osteopetrosis which is a disease associated with increased bone density and osteoclast dysfunction. After resorption is completed osteoclasts undergo programmed cell death (apoptosis), in the so-called reversal phase which heralds the start of bone formation. It has recently been discovered that many of the drugs, which are used clinically to inhibit bone resorption, such as bisphosphonates and oestrogen do so by promoting osteoclast apoptosis (see, e.g., Hughes et al., 1997).

Osteoblast Formation and Differentiation

Bone formation begins with attraction of osteoblast precursors, which are derived from mesenchymal stem cells in the bone marrow, to the bone surface. Although these cells have the potential to differentiate into many cell types including adipocytes, myocytes, and chondrocytes it is now known that the key trigger for osteoblast differentiation is expression of a regulatory molecule called Cbfa1 in pre-osteoblasts (see, e.g., Rodan et al., 1997). Cbfa1 is a transcription factor that activates co-ordinated expression of genes characteristic of the osteoblast phenotype such as osteocalcin, type I collagen and alkaline phosphatase. In contrast, expression of the transcription factor PPAR gamma promotes the cells towards adipocyte differentiation. It is currently thought that some cases of osteoporosis may occur because there is an imbalance between the rate of osteoblast and adipocyte differentiation in bone. Mature osteoblasts are plump cuboidal cells, which are responsible for the production of bone matrix. They are rich in the enzyme alkaline phosphatase and the protein osteocalcin, which are used clinically as serum markers of osteoblast activity. Osteoblasts-lay down-bone matrix which is initially unmineralised (osteoid), but which subsequently becomes calcified after about 10 days to form mature bone. During bone formation, some osteoblasts become trapped within the matrix and differentiate into osteocytes, whereas others differentiate into flattened “lining cells” which cover the bone surface. Osteocytes connect with one another and with lining cells on the bone surface by an intricate network of cytoplasmic processes, running through cannaliculi in bone matrix. Osteocytes appear to act as sensors of mechanical strain in the skeleton, and release signalling molecules such as prostaglandins and nitric oxide (NO), which modulate the function of neighbouring bone cells.

Bone remodelling is a highly organised process, but the mechanisms which determine where and when remodelling occurs are poorly understood. Mechanical stimuli and areas of micro-damage are likely to be important in determining the sites at which remodelling occurs in the normal skeleton. Increased bone remodelling may result from local or systemic release of inflammatory cytokines like interleukin-1 and tumour necrosis factor in inflammatory diseases. Calciotropic hormones such as parathyroid hormone (PTH) and 1,25-dihydroxyvitamin D, act together to increase bone remodelling on a systemic basis allowing skeletal calcium to be mobilised for maintenance of plasma calcium homeostasis. Bone remodelling is also increased by other hormones such as thyroid hormone and growth hormone, but suppressed by oestrogen, androgens and calcitonin.

Common Bone Diseases

Osteoporosis is a common disease characterised by reduced bone density, deterioration of bone tissue and increase risk of fracture. Many factors contribute to the pathogenesis of osteoporosis including poor diet, lack of exercise, smoking and excessive alcohol intake. Osteoporosis may also arise in association with inflammatory diseases such as rheumatoid arthritis, endocrine diseases such as thyrotoxicosis and with certain drug treatments such as glucocorticoids. However one of the most important factors in the pathogenesis of osteoporosis is heredity.

Paget's disease of bone is a common condition of unknown cause, characterised by increased bone turnover and disorganised bone remodeling, with areas of increased osteoclastic and osteoblast activity. Although Pagetic bone is often denser than normal, the abnormal architecture causes the bone to be mechanically weak, resulting in bone deformity and increased susceptibility to pathological fracture.

Bone involvement is a feature of many types of cancer (see, e.g., Guise & Mundy, 1998). Cancer-associated bone disease can be manifest by the occurrence of hypercalcaemia or the development of osteolytic and/or ostesclerotic metastases. Increased osteoclastic bone resorption plays a key role in the pathogenesis of both conditions. Whilst almost any cancer can be complicated by bone metastases, the most common causes are multiple myeloma, breast carcinoma, and prostate carcinoma. The most common tumours associated with hypercalcaemia are multiple myeloma, breast carcinoma, and lung carcinoma.

Known Methods of Treatment

Accelerated osteoclastic bone resorption plays an key role in the pathogenesis of common bone diseases such as osteoporosis, Paget's disease of bone, cancer-associated bone disease and periarticular bone loss in inflammatory disease states such as rheumatoid arthritis (see, e.g., Rodan et al., 2000). Because of this, most drugs which are used for the prevention and treatment of these diseases have inhibitory effects on osteoclast differentiation and/or function.

At present the most commonly used types of drugs used to suppress osteoclast activity in these diseases are bisphophonates (BPs).

Bisphosphonates (previously known as diphosphonates) are an important class of drugs used in the treatment of bone diseases involving excessive bone destruction or resorption. Bisphosphonates are structural analogues of naturally occurring pyrophosphate. Whereas pyrophosphate consists of two phosphate groups linked by an oxygen atom (P—O—P), bisphosphonates have two phosphate groups linked by a carbon atom (P—C—P). This makes bisphosphonates very stable and resistant to degradation. Furthermore, like pyrophosphate, bisphosphonates have very high affinity for calcium and therefore target to bone mineral in vivo. The carbon atom that links the two phosphate groups has two side chains attached to it, which can be altered in structure. This gives rise to a multitude of bisphosphonate compounds with different anti-resorptive potencies.

Bisphosphonates such as etidronate, clodronate, tiludronate, alendronate, risedronate, and zoledronate are highly effective agents for the treatment of osteoporosis, Paget's disease and cancer-associated bone disease. These agents can be divided into two broad classes depending on their mechanism of action.

Simple bisphosphonates such as etidronate, clodronate and tiludronate target to bone mineral and are taken up by resorbing osteoclasts. These drugs then become incorporated into non-hydrolysable analogues of adenosine triphosphate (ATP) and these metabolites inhibit osteoclast activity by interfering with essential metabolic functions (see, e.g., Frith et al., 2001; Rogers et al., 1999).

Amino bisphosphonates are also taken up by resorbing osteoclasts where they inhibit the farnesyl synthase enzyme (FPP synthase) (see, e.g., Dunford et al., 2001). This is responsible for lipid modification (prenylation) of small GTP binding proteins such as Ras, Rac, cdc42 and Rho that play a critical role in osteoclast function (see, e.g., Rogers et al., 1999). In the absence of prenylation, these signaling proteins are unable to target properly to the plasma membrane causing impairment of osteoclast function (see, e.g., Coxon et al., 2000).

Calcitonin has also been successfully used in the treatment of bone diseases such as hypercalcaemia of malignancy and osteoporosis (see, e.g., Chesnut et al., 2000; Chambers et al., 1982). Calcitonin exerts a direct inhibitory effect on bone resorption by interacting with the G-protein coupled calcitonin receptor which is highly expressed on mature osteoclasts.

Hormone replacement therapy with oestrogen is highly effective in preventing postmenopausal bone loss, but has not been studied in other diseases associated with increased osteoclastic bone resorption such as Paget's disease, hypercalcaemia and metastatic bone disease. The mechanism by which oestrogen inhibits bone resorption is incompletely understood, but it is believed to involve inhibition of expression of bone-resorbing cytokines such as IL-1, TNF and IL-6 and modulation of Osteoprotegerin and RANK ligand production within the bone microenvironment (see, e.g., Pacifici, 1996). Related compounds which act on the oestrogen receptor such as raloxifene and tibolone have similarly been used in the prevention and treatment of post-menopausal bone loss, but are not thought to be effective in treating osteoclastic bone resorption in other conditions.

Inhibitors of p38 MAP kinases have been suggested to have possible utility as inhibitors of bone resorption based on their inhibitory effects on cytokine production (see, e.g., Weier et al.). The effect of these agents on bone resorption has not been studied however and, in any case, the role of p38 MAP kinase activation in the pathogenesis of increased bone resorption in common bone diseases such as osteoporosis, Paget's disease, cancer associated bone disease and inflammation induced bone disease has not been established.

Whilst all of the above treatments are effective, each has specific drawbacks. Gastrointestinal intolerance is a problem with amino bisphosphonates and intestinal absorption of all bisphosphonates is poor. There is also a concern that long-term accumulation of bisphosphonates may occur in the skeleton, leading to impaired healing of microfractures and decreased bone quality. Calcitonin is less effective than the bisphosphonates, has to be given parenterally and has a relatively short duration of action. Hormone replacement therapy, raloxifene and tibolone are effective for the treatment of post-menopausal bone loss and osteoporosis, but not for the treatment of other bone diseases.

In view of this, there remains a clinical need for new drugs for the treatment of bone disorders which offer, for example, one or more the following benefits:

(a) improved activity;

(b) improved efficacy;

(c) improved specificity;

(d) reduced toxicity (e.g., cytotoxicity);

(e) complement the activity of other treatments (e.g., chemotherapeutic agents);

(f) reduced intensity of undesired side-effects;

(g) fewer undesired side-effects;

(h) simpler methods of administration (e.g., route, timing, compliance);

(i) reduction in required dosage amounts;

(j) reduction in required frequency of administration;

(k) increased ease of synthesis, purification, handling, storage, etc.;

(l) reduced cost of synthesis, purification, handling, storage, etc.

New Treatments

In the search for new treatments for bone disease, attention has traditionally focussed on metabolic pathways and receptor systems which affect cellular function in the bone microenvironment.

Recent studies have shown, however, that the central nervous system also plays an important role in the regulation of bone mass. For example, mice with leptin deficiency have increased bone mass which can be reversed by intracerebral infusion of low concentrations of leptin (see, e.g., Ducy et al., 2000).

The inventors realized that bone mass may be influenced by the neural pathways. This is supported by recent work that identified the neuropeptide Y2 receptor as one component of a hypothalamic relay which regulates bone mass (see, e.g., Baldock et al., 2002).

The inventor have determined that certain ligands of the endocannabinoid system (specifically, cannabinoid receptor inverse agonists and neutral antagonists) play a role in regulating osteoclast activity and bone mass, and hence are of value in the prevention and treatment of bone disorders, including those mediated by osteoclasts (e.g., increased osteoclast activity) and/or characterised by (e.g., increased) bone resorption.

Cannabinoid receptor modulators have been investigated as a possible treatment for some of the symptoms of multiple sclerosis, including spasticity and neuropathic pain; in the prevention and treatment of nausea and vomiting associated with chemotherapy; and in the treatment of anorexia associated with wasting diseases.

Localisation of CB2 receptors on cells of the immune system has led to the suggestion that cannabinoid receptor ligands may be of value as immunosuppressive and anti-inflammatory agents. Studies in animal models have shown that both CB2 selective agonists (see, e.g., Hanus et al., 1999) and CB1 selective agonists (see, e.g., Clayton et al., 2002; Smith et al., 2001) have anti-inflammatory effects. These anti-inflammatory effects are blocked by CB2 selective inverse agonists (Hanus et al, 1999; Conti et al., 2002) and by CB1 selective inverse agonists (Clayton et al., 2002) respectively.

Some publications have asserted that certain cannabinoid receptor ligands are useful in the treatment of inflammatory and autoimmune diseases; however, these documents do not refer to osteoporosis. See, for example: Inaba et al., 2003, which describes 2-oxoquinoline compounds as peripheral type cannabinoid immunomodulators; Barth et al., 1999, which describes certain 3-pyrazolecarboxamide derivatives having cannabinoid receptor affinity as potent immunomodulators; Barth et al., 2002b, which describes certain pyrazolecarboxylic acid derivatives for the treatment of obesity; Martin et al., 2003a which describes pyrazole cannabinoid agonist and antagonists.

Osteoporosis is a well-known complication of inflammatory diseases including rheumatoid arthritis, ankylosing spondylitis and inflammatory bowel disease (see, e.g., Sambrook et al., 1988; Compston et al., 1994; Croucher et al., 1993; Will et al., 1989). However, most instances of osteoporosis occurring in clinical practice are unrelated to inflammatory diseases and are instead associated with a genetic predisposition, sex hormone deficiency, or ageing. Furthermore, many of the drugs used to treat inflammatory and autoimmune diseases, such as steroids and NSAIDS, are a cause of osteoporosis, and so are contra-indicated.

Some publications have asserted, usually without any supporting evidence or data, that certain cannabinoid receptor agonists are useful in the-treatment of inflammatory and autoimmune diseases including osteoporosis or bone disease which occurs as a complication of an inflammatory disease. See, for example: Barth et al., 2002a, which describes certain 3-arylindoles as CB2 receptor agonists; Bender et al., 1998, which describes pyrazoles as CB2 receptor agonists for the treatment of immune disorders, inflammation and osteoporosis; Bender et al., 1999, which describes adamantyl phenols as cannabinoid receptor agonists in the treatment of immunologically-mediated inflammatory diseases; Kozlowski et al., 2002, which describes compounds capable of stimulating cannabinoid CB2 receptors to treat conditions characterised by inflammation immunomodulatory irregularities; Mittendorf et al., 1999, which describes agonists of the CB1 and CB2 receptor for the treatment of autoimmunologically related diseases of the bone.

These documents do not mention bone diseases associated with abnormalities of bone turnover such as Paget's disease or metastatic bone disease, or those associated with sex hormone deficiency or ageing. Furthermore, nowhere in these documents is there any teaching or suggestion that the cannabinoid receptor ligands have modulatory effects on bone formation or bone resorption, or that they might be useful in the treatment of osteoporosis which is not associated with inflammatory disease, or in the treatment of other bone diseases. It is generally recognized that such modulatory effects (either inhibition of bone resorption or stimulation of bone formation) are required for the treatment of osteoporosis and other diseases characterised by abnormalities of bone remodelling such as Paget's disease and cancer-associated bone disease.

Chackalamanil et al., 2001, and Xiang et al., 1998, assert that certain compounds, which allegedly are CB2 receptor modulators (e.g., antagonists and agonists) may have value in the treatment of inflammatory diseases and therefore be useful in the treatment of osteoporosis. No biological data is provided: instead, a prophetic assay and anticipated results are described. Xiang et al., 1998, also asserts, without any supporting evidence, that the compounds modulate bone formation/resorption (see page 12, lines 21-24 therein). Note that the “hypothesis” of both of these patents (that cannabinoid CB2 receptor antagonists would be of value in the treatment of various inflammatory conditions and associated osteoporosis) is contradicted by the teaching of many other documents (see, e.g., Bender et al., 1999, where it is asserted that cannabinoid CB2 receptor agonists would be of value in the treatment of the same range of conditions). Thus, in the absence of any supporting data, the “hypothesis” of both of these patents would not be seriously considered by the skilled worker.

At best, the above publications might suggest that cannabinoid receptor agonists might possess the desired activity, and even then, only in the context of osteoporosis associated with inflammatory diseases. None of these publications demonstrate that cannabinoid (e.g., CB1 or CB2) receptor inverse agonists or neutral antagonists have the desired therapeutic utility. Certainly none of the publications teach or suggest that cannabinoid (e.g., CB1 or CB2) receptor inverse agonists or neutral antagonists modulate osteoclast or osteoblast function, or might be useful in the treatment of osteoporosis not associated with inflammation, or in the treatment of other bone diseases.

### SUMMARY OF THE INVENTION

One aspect of the present invention pertains to use of a cannabinoid receptor inverse agonist or a cannabinoid receptor neutral antagonist for the manufacture of a medicament for the treatment of a bone disorder.

Another aspect of the present invention pertains to use of a cannabinoid receptor inverse agonist for the manufacture of a medicament for the treatment of a bone disorder

Another aspect of the present invention pertains to use of a cannabinoid receptor neutral antagonist for the manufacture of a medicament for the treatment of a bone disorder

One aspect of the present invention pertains to a method of treating a bone disorder comprising administering to a patient in need of treatment thereof a therapeutically effective amount of a cannabinoid receptor inverse agonist or a cannabinoid receptor neutral antagonist.

Another aspect of the present invention pertains to a method of treating a bone disorder comprising administering to a patient in need of treatment thereof a therapeutically effective amount of a cannabinoid receptor inverse agonist.

Another aspect of the present invention pertains to a method of treating a bone disorder comprising administering to a patient in need of treatment thereof a therapeutically effective amount of a cannabinoid receptor neutral antagonist.

One aspect of the present invention pertains to use of a compound for the manufacture of a medicament for the treatment of a bone disorder, wherein said compound is selected from compounds having a chemical formula as described herein.

One aspect of the present invention pertains to a method of treating a bone disorder comprising administering to a patient in need of treatment thereof a therapeutically effective amount of a compound selected from compounds having a chemical formula as described herein.

In one embodiment, the bone disorder is osteoporosis (e.g., osteoporosis not associated with inflammation; e.g., osteoporosis associated with a genetic predisposition, sex hormone deficiency, or ageing), cancer associated bone disease, or Paget's disease of bone.

In one embodiment, the bone disorder is: osteoporosis which is not associated with inflammation; cancer associated bone disease; or Paget's disease of bone.

In one embodiment, the bone disorder is: osteoporosis associated with a genetic predisposition, sex hormone deficiency, or ageing.

One aspect of the invention pertains to use of compounds as described herein (e.g., certain pyrazoles, etc.) for the manufacture of a medicament for the treatment of a bone disorder.

Another aspect of the present invention pertains to a method of treating a bone disorder comprising administering to a patient in need of treatment thereof a therapeutically effective amount of a compound as described herein (e.g., certain pyrazoles, etc.).

As will be appreciated by one of skill in the art, features and preferred embodiments of one aspect of the invention will also pertain to other aspects of the invention.

### BRIEF DESCRIPTION OF THE DRAWINGS

FIG. 1 is a graph showing J774 survival (% control) versus concentration of drug, for compounds AM630 (▪) and AM251 (□) after 72 hours, by Alamar Blue assay (n=3).

FIG. 2 is a bar-graph of TRAcP MNC for control, AM251 (at 10 μM) (p<0.03), and AM251 (at 20 μM) (p<0.01).

FIG. 3 is a bar-graph of resorbed area for control, AM251 (at 10 μM) (p<0.03), and AM251 (at 20 μM) (p<0.01).

FIG. 4 is a graph of TRAcP MNC versus concentration of AM251 (▪) and SR144528 (“SR144”) (Δ) (n=3).

FIG. 5 is a graph of resorbed area versus concentration of AM251 (▪) and SR144528 (“SR144”) (Δ) (n=3).

FIG. 6 is a graph of Alamar Blue J774 murine macrophage survival (% control) versus concentration of drug, for compounds AM251 (▪), SR144528 (“SR144”) (Δ) and JTE-907 (“JTE”) (●) (n=3).

FIG. 7 is a graph of osteoblast survival (% control) versus concentration of drug, for compounds SR144528 (“SR144”) (Δ), AM251 (▪) and JTE-907 (“JTE”) (●) (n=3).

FIG. 8 is a graph of Alamar Blue J774 murine macrophage survival (% control) versus concentration of drug, for three analogs of SR144528: “methyl” (♦), “piperidinyl” (▪) (n=3), and “benzodioxo” (Δ).

FIG. 9 is a bar graph showing percent changes in trabecular density, for: (a) Sham operation, no drug; (b) Sham operation, AM251 (6 mg/kg); (c) OVX operation, no drug; and (d) OVX operation, AM251 (6 mg/kg).

FIG. 10 is a bar graph showing percent changes in trabecular density, for: (a) Sham operation, no drug; (b) Sham operation, SR144528 (“SR144”) (6 mg/kg); (c) OVX operation, no drug; and (d) OVX operation, SR144528 (“SR144”) (6 mg/kg).

FIG. 11 is a bar graph showing percent changes in femoral bone mineral content, for: (a) Sham operation, no drug; (b) Sham operation, SR144528 (“SR144”) (6 mg/kg); (c) OVX operation, no drug; and (d) OVX operation, SR144528 (“SR144”) (6 mg/kg) as measured by dual energy x-ray absorptiometry.

FIG. 12 is a bar graph showing percent changes in femoral bone mineral density for (a) Sham operation, no drug; (b) Sham operation, SR144528 (“SR144”) (6 mg/kg); (c) OVX operation, no drug; and (d) OVX operation, SR144528 (“SR144”) (6 mg/kg) as measured by dual energy x-ray absorptiometry.

### DETAILED DESCRIPTION

One aspect of the present invention pertains to the use of cannabinoid receptor inverse agonists and cannabinoid receptor neutral antagonists, in the inhibition of osteoclasts (for example, the inhibition of the survival, formation, and/or activity of osteoclasts), and/or in the inhibition of bone resorption; and in connection with treatment of bone disorders, such as conditions mediated by osteoclasts (e.g., increased osteoclast activity) and/or characterised by (e.g., increased) bone resorption.

Cannabis and Cannabinoids

The plant species Cannabis sativa L., also known as cannabis, marijuana, and Indian hemp, is of the genus Cannabis L. (hemp) and the family Cannabaceae (also Cannabidaceae) (hemp family). Two sub-species are known, ssp. indica and ssp. sativa, as well as several varieties of the latter (e.g., Purple Haze).

Cannabis is a source of fiber (hemp), oil, medicines, and narcotics (psychotropics). Most varieties contain biologically active terpenoid derivatives, such as cannabinol, isomeric tetrahydrocannabinols, and cannabidiol, collectively referred to as “cannabinoids.” A variety of derivatives and analogs of these compounds have been prepared and studied. Both the natural product Δ⁹-THC (also known as Dronabinol® and Marinol®) and the synthetic analogue Cesamet® (also known as Nabilone®) are licensed for use in the United Kingdom as antiemetics. See, for example, Goutopoulos et al., 2002.

Cannabinoid Binding Sites

Specific cannabinoid binding sites for exogenous cannabinoids were first demonstrated in 1988. Since then, two distinct cannabinoid binding site receptors have been identified: the CB1 receptor and the CB2 receptor. CB1 is a ubiquitous receptor found in the central nervous system (CNS) and the periphery, and in both neural and non-neural tissues. The CB2 receptor has a more limited distribution, principally in cells associated with the immune system. Recently, another cannabinoid receptor has been characterised in the brain which binds anandamide and SR141716A, but not other cannabinoid receptor ligands (see, e.g., Breivogel et al., 2001). In addition, SR144528 may interact with a CB2-like receptor located on immune cells (Pertree and Ross, 2002).

The endogenous cannabinoid (endocannabinoid) system comprises at least two receptors (CB1 and CB2), each with different localisations and functions; a family of endogenous ligands; and a specific molecular machinery for the synthesis, transport, and inactivation of these ligands. This system has been shown to have a huge range of effects in the nervous, immune and cardiovascular systems (see, e.g., Lichtman et al., 2002; Parolaro et al., 2002; Rice et al., 2002).

The existence of the CB1 and CB2 binding sites strongly suggested the existence of one or more endogenous ligands (endogenous cannabinoids, endocannabinoids) that exert their physiological activity upon binding to these receptors.

In 1992, the first endogenous ligand (endogenous cannabinoid, endocannabinoid), arachidonyl ethanolamide (AEA), also known as anandamide, which binds to CB1, was isolated from human brain tissue. Subsequently, a number of endogenous cannabinoids (e.g., such as those shown below) have been identified and shown to be involved in the control of various physiological functions including pain transmission, inflammation, appetite, motor function, learning and memory (see, e.g., Pertwee et al., 2002).

Cannabinoid receptor modulators are currently being investigated as a possible treatment for some of the symptoms of multiple sclerosis, neuropathic and inflammatory pain, the prevention and treatment of nausea and vomiting associated with chemotherapy and the treatment of anorexia associated with wasting diseases.

Localisation of CB2 receptors on cells of the immune system has led to the suggestion that cannabinoid agonists may also play a role as immunosuppressive and anti-inflammatory agents. In fact, CB2 receptors have been implicated in the anti-inflammatory actions of endocananbinoids and a CB2-selective agonist has been shown to be a potent anti-inflammatory compound (see, e.g., Hanus et al., 1999).

Using DNA microarray technology it has recently been shown that, activation of CB2 receptors in promyelocytic cells (HL-60) induces an up-regulation of 5 genes involved in cytokine production and regulation (IL-8, MCP-1, MIP-1β, TNFα, A20) and 4 genes involved in transcription and cell cycling (Jun B, Aldose C, BTG2, IκB-α) (Derocq et al, 2000). These changes are highly sensitive to cannabinoids, since significant alterations in gene expression are induced by low concentrations of agonist (10 nM). The transcriptional events reported are implicated in the cell differentiation program and suggest that CB2 receptors are important in control of the initialisation of cell maturation. Furthermore, CB2 receptor activation appears to induce conditions that promote the transition of HL-60 cells to a more monocytic/granulocytic phenotype. In addition to the observed agonist-induced enhancement of mRNA expression, a decrease in the basal levels mRNA expression was observed in the presence of the inverse agonist SR144528.

Despite the pleiotropic effects of cannabinoids, there have been no prior suggestions or data to support the hypothesis that the endocannabinoid system may play a role in bone metabolism.

Cannabinoid Receptor Inverse Agonists and Neutral Antagonists

Thus, one aspect of the present invention pertains to use of a cannabinoid receptor inverse agonist or a cannabinoid receptor neutral antagonist for the manufacture of a medicament for the treatment of a bone disorder.

Another aspect of the present invention pertains to a method of treating a bone disorder comprising administering to a patient in need of treatment thereof a therapeutically effective amount of a cannabinoid receptor inverse agonist or a cannabinoid receptor neutral antagonist.

In one embodiment, the cannabinoid receptor is CB1.

In one embodiment, the cannabinoid receptor is CB2.

In one embodiment, the cannabinoid receptor is CB1 or CB2.

In one embodiment, the cannabinoid receptor inverse agonist or cannabinoid receptor neutral antagonist is a CB1 inverse agonist or a CB1 neutral antagonist.

In one embodiment, the cannabinoid receptor inverse agonist or cannabinoid receptor neutral antagonist is a CB2 inverse agonist or a CB2 neutral antagonist.

In one embodiment, the cannabinoid receptor inverse agonist or cannabinoid receptor neutral antagonist is a CB1 inverse agonist or a CB1 neutral antagonist or a CB2 inverse agonist or a CB2 neutral antagonist.

In one embodiment, the cannabinoid receptor inverse agonist or cannabinoid receptor neutral antagonist is a CB1 inverse agonist or a CB1 neutral antagonist; and is also a CB2 inverse agonist or a CB2 neutral antagonist.

In one embodiment, the cannabinoid receptor inverse agonist or cannabinoid receptor neutral antagonist is a CB1 inverse agonist or a CB1 neutral antagonist; but is not a CB2 inverse agonist or a CB2 neutral antagonist.

In one embodiment, the cannabinoid receptor inverse agonist or cannabinoid receptor neutral antagonist is a CB2 inverse agonist or a CB2 neutral antagonist; but is not a CB1 inverse agonist or a CB1 neutral antagonist.

Thus, one aspect of the present invention pertains to use of a cannabinoid receptor inverse agonist for the manufacture of a medicament for the treatment of a bone disorder Another aspect of the present invention pertains to a method of treating a bone disorder comprising administering to a patient in need of treatment thereof a therapeutically effective amount of a cannabinoid receptor inverse agonist.

In one embodiment, the cannabinoid receptor inverse agonist is a CB1 inverse agonist.

In one embodiment, the cannabinoid receptor inverse agonist is a CB2 inverse agonist.

In one embodiment, the cannabinoid receptor inverse agonist is a CB1 inverse agonist or a CB2 inverse agonist.

In one embodiment, the cannabinoid receptor inverse agonist is a CB1 inverse agonist; and is also a CB2 inverse agonist or a CB2 neutral antagonist.

In one embodiment, the cannabinoid receptor inverse agonist is a CB2 inverse agonist; and is also a CB1 inverse agonist or a CB1 neutral antagonist.

In one embodiment, the cannabinoid receptor inverse agonist is a CB1 inverse agonist; but is not a CB2 inverse agonist or a CB2 neutral antagonist.

In one embodiment, the cannabinoid receptor inverse agonist is a CB2 inverse agonist; but is not a CB1 inverse agonist or a CB1 neutral antagonist.

Thus, one aspect of the present invention pertains to use of a cannabinoid receptor neutral antagonist for the manufacture of a medicament for the treatment of a bone disorder

Another aspect of the present invention pertains to a method of treating a bone disorder comprising administering to a patient in need of treatment thereof a therapeutically effective amount of a cannabinoid receptor neutral antagonist.

In one embodiment, the cannabinoid receptor neutral antagonist is a CB1 neutral antagonist.

In one embodiment, the cannabinoid receptor neutral antagonist is a CB2 neutral antagonist.

In one embodiment, the cannabinoid receptor neutral antagonist is a CB1 neutral antagonist or a CB2 neutral antagonist.

In one embodiment, the cannabinoid receptor neutral antagonist is a CB1 neutral antagonist; and is also a CB2 inverse agonist or a CB2 neutral antagonist.

In one embodiment, the cannabinoid receptor neutral antagonist is a CB2 neutral antagonist; and is also a CB1 inverse agonist or a CB1 neutral antagonist.

In one embodiment, the cannabinoid receptor neutral antagonist is a CB1 neutral antagonist; but is not a CB2 inverse agonist or a CB2 neutral antagonist.

In one embodiment, the cannabinoid receptor neutral antagonist is a CB2 neutral antagonist; but is not a CB1 inverse agonist or a CB1 neutral antagonist.

In one embodiment, the cannabinoid receptor inverse agonist or cannabinoid receptor neutral antagonist is (additionally) CB1 selective.

In one embodiment, the cannabinoid receptor inverse agonist or cannabinoid receptor neutral antagonist is (additionally) CB2 selective.

Cannabinoid (CB) Receptor Ligands—Affinity

In general, a particular ligand which binds to a particular receptor is said to have affinity for that receptor.

A measure of affinity is often determined using a binding assay, for example, a competition or displacement assay, in which a candidate ligand competes with, or displaces, a known (or reference) ligand with a known (or reference) affinity. Such assays yield an inhibition constant (Ki) for the candidate ligand. The Ki value is inversely proportional to the affinity of the candidate ligand for the receptor. Thus, a low Ki value signifies a high affinity. In general, a Ki value of 10 μM (10,000 nM) or less is considered to be a meaningful affinity for the receptor, and indicates that the candidate compounds is in fact a ligand for that receptor.

Assays for determining cannabinoid receptor affinity are well known. See, for example, Ross et al., 1999a; Ross et al., 1999b; Huffman et al., 2000; Huffman et al., 2001. For example, radio-ligand displacement assays using tissues that contain the CB1 receptor (brain, CB1 transfected cell lines) or the CB2 receptor (spleen, CB2 transfected cell lines) are common. Examples of suitable radio-labelled known (reference) ligands include tritium-labeled SR14716A (a CB1 specific receptor inverse agonist), tritium-labeled CP55940 (a CB1/CB2 receptor agonist). One suitable method is described below.

In one embodiment, the cannabinoid receptor inverse agonist or cannabinoid receptor neutral antagonist has a cannabinoid receptor inhibition constant (Ki) of 10 μM or less.

In one embodiment, range is: 1 μM or less; 500 nM or less; 100 nM or less; 50 nM or less; 25 nM or less; 10 nM or less; 5 nM or less; 2 nM or less; or 1 nM or less.

In one embodiment, the range is: from 0.001 nM to 10 μM; from 0.001 nM to 1 μM; from 0.001 nM to 500 nM; from 0.001 nM to 100 nM; from 0.001 nM to 50 nM; from 0.001 nM to 25 nM; from 0.001 nM to 10 nM; from 0.001 nM to 5 nM; from 0.001 nM to 2 nM; or from 0.001 nM to 1 nM.

In one embodiment, the cannabinoid receptor inverse agonist or cannabinoid receptor neutral antagonist has a CB1 inhibition constant (Ki) as defined above.

In one embodiment, the cannabinoid receptor inverse agonist or cannabinoid receptor neutral antagonist has a CB2 inhibition constant (Ki) as defined above.

In one embodiment, the a cannabinoid receptor inverse agonist or a cannabinoid receptor neutral antagonist has a CB1 inhibition constant (Ki) as defined above, and a CB2 inhibition constant (Ki) as defined above.

Cannabinoid Receptor Binding Assays

Cannabinoid receptor binding (and thus ligand affinity) can readily be determined by looking for displacement of a suitable known ligand by a test ligand from mouse brain and spleen membranes. Examples of suitable known ligands include tritium labeled SR141716A (a CB1-specific receptor inverse agonist) and CP55940 (a CB1/CB2 receptor agonist).

One suitable method is described next. MF1 mice are killed by cervical dislocation and the desired tissues (brain and spleen) dissected out and placed into cold centrifugation buffer (320 mM sucrose, 2 mM Tris EDTA, 5 mM MgCl₂) on ice. Tissue is then homogenized with an ultra-turrax polytron homogeniser. The homogenate is centrifuged at 1600×g for 10 minutes, the supernatant saved on ice and the pellet re-suspended in cold centrifugation buffer and centrifuged at 1600×g for 10 minutes. The supernatants are combined and centrifuged at 32000×g for 20 minutes. This supernatant is discarded and the pellet resuspended in 50 mL of Tris Buffer (50 mM Tris, 2 mM EDTA and 5 mM MgCl₂), incubated at 37° C. for 10 minutes and centrifuged at 23000×g for 20 minutes. The final pellet is resuspended in 40 mL Tris Buffer and left to stand at room temperature for 40 minutes. This solution is then centrifuged at 11000×g for 15 minutes and the pellet resuspended in assay buffer (1 mM MgCl₂, 50 mM Tris, 1 mM EDTA) to a concentration of 1 mg/mL as determined by the Lowry method (Bio-Rad Dc kit).

Radioligand binding assays are performed, for example, with the CB1 receptor inverse agonist [³H]SR141716A (0.5 nM) (brain membranes) or [³H]CP55940 (0.5 nM) (spleen membranes) in assay buffer containing 1 mg/mL BSA, the total assay volume being 500 μL. Binding is initiated by the addition of membranes (100 μg). The vehicle concentration of 0.1% DMSO is kept constant throughout. Assays are carried out at 37° C. for 60 minutes before termination by addition of ice-cold wash buffer (50 mM Tris buffer, 1 mg/mL BSA) and vacuum filtration using a 12-well sampling manifold (Brandel Cell Harvester) and Whatman GF/B glass-fibre filters that had been soaked in wash buffer at 4° C. for 24 hours. Each reaction tube is washed five times with a 4 mL aliquot of buffer. The filters are oven-dried for 60 minutes and then placed in 5 mL of scintillation fluid (Ultima Gold XR, Packard), and radioactivity quantitated by liquid scintillation spectrometry.

Specific binding is defined as the difference between the binding that occurred in the presence and absence of 1 μM unlabelled ligand and reported as a percentage of the total radio-ligand bound in brain and spleen respectively.

The concentrations of competing ligands (test compounds) to produce 50% displacement of the radioligand (IC50) from specific binding sites is calculated, for example, using GraphPad Prism (GraphPad Software, San Diego). Inhibition constant (Ki) values are calculated using the equation of Cheng & Prusoff (see, e.g., Cheng et al., 1973).

Cannabinoid (CB) Receptor Ligands—Functional Characteristics (1)

Although binding studies measure the affinity of a ligand for the receptor, such studies do not indicate the functional characteristics of the ligand (that is, whether it acts as an agonist, neutral antagonist, inverse agonist, etc.).

Thus, many cannabinoid receptor ligands may also be conveniently classified according to their functional characteristics, for example, their effect upon cannabinoid receptor activity, for example, as an agonist, neutral antagonist, inverse agonist, etc.

Both CB1 and CB2 receptors belong to the G protein-coupled receptor (GPCR) super-family and are coupled to inhibition of adenylyl cyclase and activation of extracellular signal-regulated cascade (ERK). See, e.g., the review by Pertwee, 2001.

The traditional model of G protein-coupled receptor (GPCR) action is based on the premise that the binding of an agonist to the receptor is necessary for receptor activation. However, it is now clear that some receptor activation occurs spontaneously, without agonist binding, the receptors being “constitutively active.”

Cannabinoid CB1 and CB2 receptors appear to be constitutively active. A large body of evidence for this has been obtained from high expression recombinant cell lines where cannabinoid receptor inverse agonists stimulate adenylyl cyclase and inhibit ERK (see, e.g., Bouaboula et al., 1996; Bouaboula et al., 1997; Bouaboula et al., 1999). By sequestration of Gi proteins, cannabinoid inverse agonists not only inhibit constituitively active CB1/CB2 receptors but also inhibit receptor activation by other unrelated Gi-dependent receptors (see, e.g., Bouaboula et al., 1999).

In general, ligands that do not bind directly to a receptor, but do affect the receptor's function, may be described as “modulators.” There are numerous examples of so-called allosteric modulators of G-protein coupled receptors that bind to a site closely related to the receptor and modulate the function of the receptor (see, e.g., Vaulquelin et al., 2002). Such sites may exist for the cannabinoid receptors; however, none have yet been identified.

Thus, many cannabinoid receptor ligands may be further classified as:

(a) cannabinoid receptor agonists, which activate the receptor; partial agonists also activate the receptor, but with lower efficacy than a full agonist;

(b) cannabinoid receptor inverse agonists, which both block the action of the agonist and attenuate receptor-constitutive activity;

(c) cannabinoid receptor neutral antagonists, which block the action of the agonist but are ineffective on the receptor-constitutive activity; they may also be low efficacy partial agonists that behave as antagonists.

Examples of (a) cannabinoid receptor agonists include, but are not limited to:

L-759633, which is a CB2 selective agonist (see, e.g., Gareau et al., 1996; Ross et al., 1999a);

L-759666, which is a CB2 selective agonist (see, e.g., Gareau et al., 1996; Ross et al., 1999a);

JWH-133, which is a CB2 selective agonist (see, e.g., Huffman et al., 2001);

HU308, which is a CB2 selective agonist (see, e.g., Hanus et al., 1999).

Δ⁹-THC, which has considerably lower efficacy at the CB2 receptor than CB1, and has been reported to behave as a CB2 receptor antagonist (see, e.g., Bouaboula et al., 1999);

O-1238, which is a non-selective partial agonist (see, e.g., Ross et al., 1999b). Anandamide, which is a partial agonist at the CB2 receptor (see Pertwee, 1999).

Examples of (b) cannabinoid receptor inverse agonists include, but are not limited to:

SR141716A, which is CB1 selective (see, e.g., Bouaboula et al., 1997);

AM281, which is CB1 selective (see, e.g., Gifford et al., 1997; Lan et al., 1999);

AM251, which is CB1 selective (see, e.g., Lan et al., 1999);

LY321035, which is CB1 selective (see, e.g., Felder et al., 1998);

SR144528, which is CB2 selective (see, e.g., MacLennan et al., 1998);

JTE-907, which is CB2 selective (see, e.g., Iwamura et al., 2001);

O-1184 which has equal affinity for CB1 and CB2 receptors but is a CB2 receptor inverse agonist (see, e.g., Ross et al., 1999b).

Examples of candidates for (c) cannabinoid receptor neutral antagonists include, but are not limited to:

cannabinol, which is a CB1 receptor antagonist (see, e.g., MacLennan et al., 1998);

Δ⁹-THC, which has considerably lower efficacy at the CB2 receptor than CB1, and has been reported to behave as a CB2 receptor antagonist (see, e.g., Bouaboula et al., 1999);

O-1238, a non-selective partial agonist (see, e.g., Ross et al., 1999b).

Some examples of cannabinoid receptor agonists, neutral antagonists, and partial agonists are listed in Table 1.

TABLE 1
Compounds that display partial agonist, neutral antagonist and inverse
agonist activity at CB1 or CB2 receptors.

| | Ki (nM) | | Classification | |
|---|---|---|---|---|
| Ligand | CB1 | CB2 | CB1 | CB2 |
| SR141716A | 12.3 | 702 | Inverse agonist | Inverse agonist |
| LY320135 | 141 | 14900 | Inverse agonist | Inactive |
| AM281 | 12 | 4200 | Inverse agonist | Status unclear |
| AM251 | 0.5 | 2000 | Inverse agonist | Status unclear |
| SR144528 | 437 | 5.6 | Inverse agonist | Inverse agonist |
| AM630 | 5152 | 31.2 | Inverse agonist | Inverse agonist |
| JTE-907 | 684 | 1.55 | Inactive | Inverse agonist |
| O1184 | 5.2 | 7.4 | Partial agonist | Inverse agonist |
| O1238 | 3.5 | 7.8 | Partial agonist | Partial agonist |
| Cannabinol | 115 | 580 | Neutral antagonist | Inverse agonist |
| Δ⁹THC | 41 | 36 | Partial agonist | Neutral antagonist |
| Anandamide | 89 | 371 | Agonist | Partial agonist |

While the compounds in the above Table have been extensively characterised, many more compounds with affinity for CB1 and CB2 receptors have been less well characterised. While radioligand binding experiments have confirmed that such compounds have selectivity for either the CB1 or CB2 receptor, some have not yet been classified as being agonists, neutral antagonists, inverse agonists, etc.

Cannabinoid (CB) Receptor Ligands—Functional Characteristics (2)

Cannabinoid receptor ligands may be functionally characterised, for example, according to:

(1) their effect upon adenylyl cylase activity; and/or

(2) their effect upon [³⁵S]-γ-GTP binding.

Thus, many cannabinoid receptor ligands may be further classified as:

(A) cannabinoid receptor agonists, which:

(i) inhibit adenylyl cyclase activity.

Inhibition of adenylyl cyclase is measured using a cyclic AMP assay (see below). Certain compounds will cause formation of cyclic AMP (i.e., stimulate cyclic AMP production) in cells and tissues. One such compound is forskolin. The stimulation of cyclic AMP production by forskolin is inhibited by cannabinoid receptor agonists. The cyclic AMP assay will yield an IC50 (see methods) for cannabinoid receptor agonists. The level of inhibition of forskolin-stimulated cyclic AMP production is expressed as a percent (%) of the cyclic AMP production induced by forskolin alone. The concentration of cannabinoid receptor ligand which produces 50% inhibition (IC50) of forskolin-stimulated cyclic AMP production is calculated using GraphPad Prism (GraphPad Software, San Diego). If a cannabinoid receptor ligand has an IC50 value for inhibition of forskolin-stimulated cyclic AMP production of from 0.001 nM to 10 μM, then it is considered to be a cannabinoid receptor AGONIST.

(ii) Stimulate [³⁵S]-γ-GTP Binding.

Agonist activation of a G-protein coupled receptor by a compound causes GTP to attach to the receptor. In this assay, the GTP is radiolabelled ([³⁵S]-γ-GTP) and thus the amount of GTP linked to the receptor can be measured. The amount of GTP binding to the receptor is directly proportional to the level of activation of the receptor. The [³⁵S]-γ-GTP binding assay measures the amount of radioactivity bound to cells and tissues. The assay will yield an EC50 value for cannabinoid receptor agonists (see methods). The [³⁵S]-γ-GTP bound in the presence of a cannabinoid receptor agonist will increase and is expressed as a percent (%) of the specific binding. The % stimulation at each concentration of agonist is calculated and a concentration-response curve drawn using Prism (GraphPad). The concentration of agonist producing 50% stimulation of [³⁵S]-γ-GTP binding is defined as the EC50. The Emax value is the maximum response to a given agonist. If a cannabinoid receptor ligand has an EC50 value of from 0.001 nM to 10 μM for stimulation of [³⁵S]-γ-GTP binding, then it is considered to be an AGONIST.

or:

(B) cannabinoid receptor inverse agonists, which:

(i) stimulate adenylyl cylase activity.

Inhibition of adenylyl cyclase is measured using a cyclic AMP assay (see below). Certain compounds will cause formation of cyclic AMP (i.e., stimulate cyclic AMP production) in cells and tissues. One such compound is forskolin. The stimulation of cyclic AMP production by forskolin is enhanced by cannabinoid receptor inverse agonists.

Cannabinoid receptor inverse agonists will also stimulate the production of cyclic AMP in the absence of forskolin: A cannabinoid receptor inverse agonist will enhance forskolin-stimulated cyclic AMP production. A graph of this enhancement is drawn using GraphPad Prism (GraphPad Software, San Diego) and the EC50 is the concentration of cannabinoid receptor ligand that produces a 50% stimulatory response. If a cannabinoid receptor ligand has an EC50 value for stimulation of cyclic AMP production of from 0.001 nM to 10 μM, then it is considered to be a cannabinoid receptor INVERSE AGONIST.

(ii) inhibit [³⁵S]-γ-GTP binding.

Inverse agonist activation of a G-protein coupled receptor by a compound causes GTP to detach from the receptor. In this assay, the GTP is radiolabeled ([³⁵S]-γ-GTP) and thus the amount of GTP linked to the receptor can be measured. The [³⁵S]-γ-GTP binding assay measures the amount of radioactivity bound to cells and tissues. The assay will yield an IC50 value for cannabinoid receptor inverse agonists (see methods). The % inhibition is calculated for each concentration of compound and calculated and a concentration-response curve drawn using Prism (GraphPad). The concentration of inverse agonist producing 50% inhibition of [³⁵S]-□-GTP binding is defined as the IC₅₀. If a cannabinoid receptor ligand has an IC50 value of from 0.001 nM to 10 μM for inhibition of [³⁵S]-γ-GTP binding, then it is considered to be an INVERSE AGONIST.

or:

(c) cannabinoid receptor neutral antagonists, which:

(i) block the inhibition of adenylyl cylase activity by cannabinoid receptor agonists.

As described in (A) above, the stimulation of cyclic AMP production by forskolin is inhibited by cannabinoid receptor agonist. The cyclic AMP assay will yield an IC50 (see methods) for cannabinoid receptor agonists. A neutral antagonist will have no effect upon cyclic AMP production when added to cells or tissues alone. A neutral antagonist will block the inhibition of cyclic AMP production observed with an agonist (as described in (A) above). A neutral antagonist will cause the IC50 for an agonist to be increased. The ratio of the IC50 value in the presence and absence of an antagonist is referred to as the “dose ratio” (DR). The following formula is used to calculate the Kb value for the antagonist, where B is the concentration of antagonist: (DR−1)=(B)(Kb). The Kb value is a measure of the ability of the compound to antagonise the activation of the receptor by the agonist. A cannabinoid receptor ligand with a Kb value of from 0.001 nM to 10 μM would be considered to be an antagonist. Note that both inverse agonists and antagonists will block the effect of agonists, but a neutral antagonist will NOT stimulate the production of cyclic AMP.

(ii) block the stimulation of [³⁵S]-γ-GTP binding by a cannabinoid receptor agonist.

A neutral antagonist interacting with a G-protein coupled receptor will have no effect upon the GTP bound to the receptor. In this assay, the GTP is radiolabelled ([³⁵S]-γ-GTP) and thus the amount of GTP linked to the receptor can be measured. The [³⁵S]-γ-GTP binding assay measures the amount of radioactivity bound to cells and tissues. A neutral antagonist will block the stimulation of [³⁵S]-γ-GTP binding observed with an agonist (as described in (A) above). A neutral antagonist will cause the EC50 for an agonist to be increased. The ratio of the EC50 value in the absence and presence of an antagonist is referred to as the “dose ratio” (DR). The following formula is used to calculate the Kb value for the antagonist, where B is the concentration of antagonist: (DR−1)=(B)(Kb). The Kb value is a measure of the ability of the compound to antagonise the activation of the receptor by the agonist. A cannabinoid receptor ligand with a Kb value of from 0.001 nM to 10 μM would be considered to be an antagonist. Note that both inverse agonists and antagonists will block the effect of agonists, but a neutral antagonist will NOT inhibit [³⁵S]-γ-GTP binding.

Thus:

In one embodiment, the cannabinoid receptor inverse agonist stimulates cyclic AMP production with an EC50 value of 10 μM or less.

In one embodiment, the cannabinoid receptor inverse agonist inhibits [³⁵S]-γ-GTP binding with an IC50 value of 10 μM or less.

In one embodiment, both of the above apply.

In one embodiment, the cannabinoid receptor neutral antagonist does not affect cyclic AMP production, but blocks the inhibition of cyclic AMP production by a cannabinoid receptor agonist with a Kb value of 10 μM or less.

In one embodiment, the cannabinoid receptor neutral antagonist does not affect the binding of [³⁵S]-γ-GTP, but does block the stimulation of [³⁵S]-γ-GTP binding by an agonist with a Kb value of 10 μM or less.

In one embodiment, both of the above apply.

In one embodiment, range is: 1 μM or less; 500 nM or less; 100 nM or less; 50 nM or less; 25 nM or less; 10 nM or less; 5 nM or less; 2 nM or less; or 1 nM or less.

In one embodiment, the range is: from 0.001 nM to 10 μM; from 0.001 nM to 1 μM; from 0.001 nM to 500 nM; from 0.001 nM to 100 nM; from 0.001 nM to 50 nM; from 0.001 nM to 25 nM; from 0.001 nM to 10 nM; from 0.001 nM to 5 nM; from 0.001 nM to 2 nM; or from 0.001 nM to 1 nM.

Cyclic AMP Assay

Cannabinoid receptors CB1 and CB2 are coupled to inhibition of adenylyl cyclase (see, e.g., Bidault-Russell et al., 1990; Childers et al., 1996). Adenylyl cyclase is an enzyme that catalyses the production of cyclic adenosine monophosphate (AMP). Thus, activation of the receptor leads to the inhibition of the production of cyclic AMP. Certain compounds, such as forskolin, stimulate adenylyl cyclase. Accumulation of cyclic AMP is then measured using a radio-immunoassay, and is indicative of adenylyl cyclase activation. The radioimmunoassay uses radiolabelled cyclic AMP. The amount of radioactivity can be measured and will be proportional to the level of cyclic AMP that is produced. The cyclic AMP assay is performed with a phosphodiesterase inhibitor present. This is necessary because phosphodiesterase is an enzyme that rapidly breaks down cyclic AMP. An example of a phosphodiesterase inhibitor is rolipram. The cyclic AMP assay is performed using cells that contain CB1 receptors only or cells that contain CB2 receptors only (Chinese Hamster Ovary Cells or Human Embryonic Kidney Cells, respectively). The cyclic AMP assay may also be also performed with tissues that contain CB1 receptors (e.g., brain) or CB2 receptors (e.g., spleen).

The cells or tissues are incubated for 30 minutes at 37° C. with the cannabinoid receptor ligand and the phosphodiesterase inhibitor rolipram (Sigma) (50 μM) in phosphate buffered saline (PBS) containing 1 mg/ml bovine serum albumin (Sigma). The cells or tissues are then incubated for a further 30 minutes incubation with 2 μM forskolin (Sigma). The reaction is terminated by addition 0.1 M hydrochloric acid and the mixture is centrifuged in a microfuge to remove cell debris. The resulting pellet contains cell debris and the supernatant contains the [³H] cyclic AMP. A sample of a supernatant is removed and the pH is adjusted to pH 8-9 using 1 M NaOH. The cyclic AMP content is then measured using a radioimmunoassay kit ([³H] Biotrack assay TRK432, from Amersham Biosciences), following the manufacturers instructions. The amount of radioactivity in each sample is counted using a Beckman scintillation counter. The amount is cyclic AMP in each sample is calculated from the level of radioactivity.

[³⁵S]-γ-GTP Assay

Activation of a G-protein coupled receptor by an agonist leads to the replacement of guanosine diphosphate (GDP) with guanosine triphosphate (GTP). The level of binding of GTP to the receptor is proportional to the level of receptor activation. The level of binding is measured by using a radiolabelled from of GTP called [³⁵S]-γ-GTP. Thus the radioactivity can be measured and is proportional to the amount of GTP bound to the receptor. The [³⁵S]-γ-GTP binding assay is performed with cells that contain CB1 receptors only or cells that contain CB2 receptors only (Chinese Hamster Ovary cells or human embryonic kidney cells, respectively). The [³⁵S]-γ-GTP binding assay may also be performed with tissues that contain CB1 receptors (e.g., brain) or CB2 receptors (e.g., spleen).

Cells (see above) that contain CB1 or CB2 receptors only are removed from flasks by scraping, and are re-suspended in homogenisation buffer (0.32 M sucrose/50 mM Tris), and homogenised using an Ultra-Turrex homogeniser. If tissues are used, the homogenate is prepared as for a radioligand binding assay (see above). The homogenate is diluted with Tris buffer (50 mM, pH 7.4) and centrifuged at 50,000×g for 45 minutes. Cell membranes (20 μg) (see above) are incubated in assay buffer containing 2 mg/ml fatty acid free bovine serum albumin (BSA), 20 μM GDP, and 0.1 nM [³⁵S]-γ-GTP (New England Nuclear). The assay buffer contains: 50 mM Tris; 10 mM MgCl₂; 100 mM NaCl; 0.2 mM EDTA at pH 7.4. Incubation times are for 90 minutes at 30° C. The reaction is terminated by the addition of 4 mL of ice-cold wash buffer (50 mM Tris, 1 mg/mL BSA, pH 7.4) followed by rapid filtration under vacuum through Whatman GF/B glass fibre filters using a 12-tube Brandel cell harvester. The filters are washed 3 times with 4 mL of wash buffer. The filters are then dried, placed in scintillation fluid, and bound radioactivity is determined by liquid scintillation counting and reported, e.g., in units of disintegrations per minute (dpm). The binding of [³⁵S]-γ-GTP is determined (a) in the presence of 20 μM GDP (this is the “total binding”, TB), and (b) in the presence of 10 μM S-γ-GTP (this is the “non-specific binding”, NSB). The level of binding of [³⁵S]-γ-GTP is reported as a percentage change with respect to basal levels. The “specific” binding (SB) of [³⁵S]-γ-GTP to the receptor is defined as the total binding less the non-specific binding (i.e., SB=TB−NSB), and this value is taken as 100%.

Cannabinoid (CB) Receptor Ligands—Chemical Structure

Cannabinoid receptor ligands may also be conveniently classified according to chemical structure, for example, as discussed below.

Many of these classes, and their-members, are also cannabinoid receptor inverse agonists or cannabinoid neutral antagonists, as described herein, and are suitable for use in the present invention.

Thus, one aspect of the invention pertains to use of such compounds for the manufacture of a medicament for the treatment of a bone disorder.

Another aspect of the present invention pertains to a method of treating a bone disorder comprising administering to a patient in need of treatment thereof a therapeutically effective amount of such compounds.

A. Classical Cannabinoids

This class of ligands includes those which are structurally similar to Δ⁹-THC and have a (partially hydrogenated) 10aH-benzo[c]chromene core.

Thus, one aspect of the invention pertains to use of a compound (e.g., a cannabinoid receptor inverse agonist or a cannabinoid receptor neutral antagonist) for the manufacture of a medicament for the treatment of a bone disorder, wherein the compound is Δ⁹-THC or an analogue or derivative thereof.

Another aspect of the present invention pertains to a method of treating a bone disorder comprising administering to a patient in need of treatment thereof a therapeutically effective amount of a compound (e.g., a cannabinoid receptor inverse agonist or a cannabinoid receptor neutral antagonist) that is Δ⁹-THC or an analogue or derivative thereof.

Examples of such compounds include, but are not limited to, the following:

| Compound | Structure |
|---|---|
| Δ⁹-tetrahydrocannabinol(⁻Δ⁹-THC) =Δ¹-3,4-trans-isomer(Δ¹-THC) | 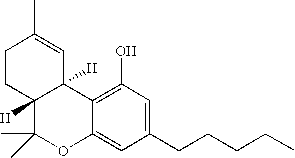 |
| Δ⁸-tetrahydrocannabinol(Δ⁸-THC) =Δ⁶-3,4-trans-isomer(Δ⁶-THC) | 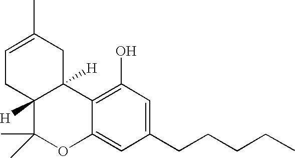 |
| cannabinol | 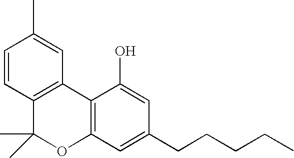 |
| Cesamet (Nabilone) | 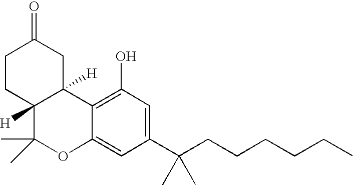 |
| HU-210 | 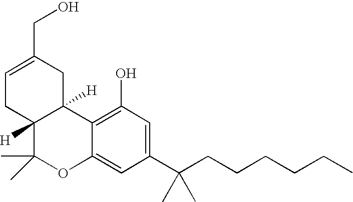 |
| dehydroxy-HU-210 | 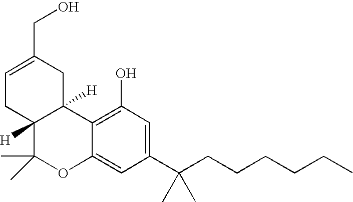 |
| AMG-3 | 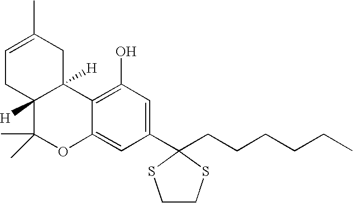 |
| AM-919 | 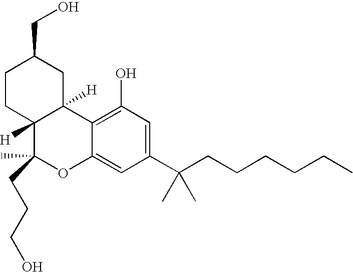 |
| O-1057 | 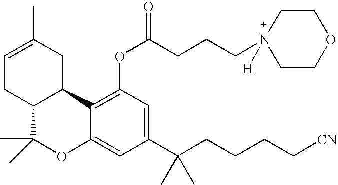 |
| JWH-140 | 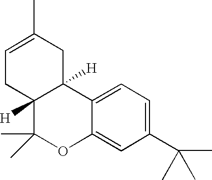 |
| JWH-139 | 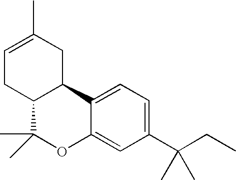 |
| JWH-133 | 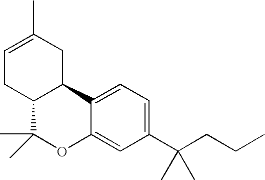 |
| JWH-065 | 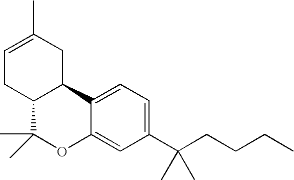 |
| JWH-066 | 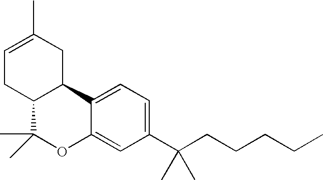 |
| JWH-067 | 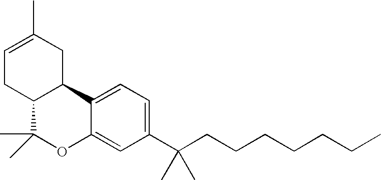 |
| JWH-068 | 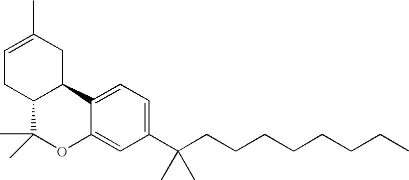 |
| JWH-051 | 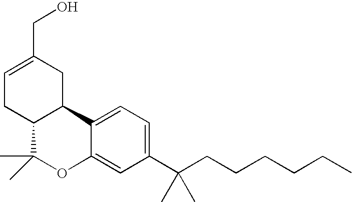 |
| JWH-057 | 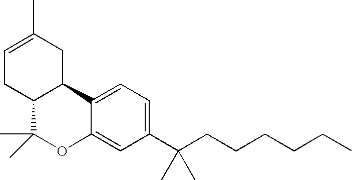 |
| O-1184 | 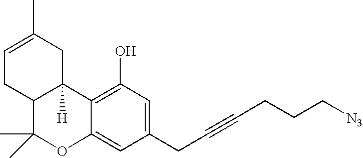 |
| O-1238 | 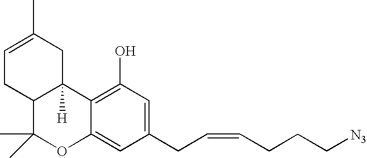 |
| L759633 | 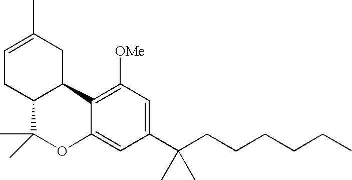 |
| L759656 | 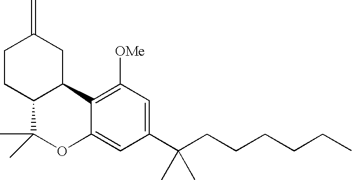 |
| JWH-101 | 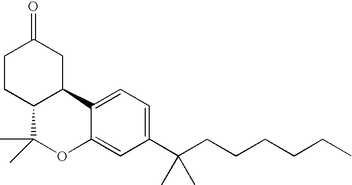 |
| JWH-104 | 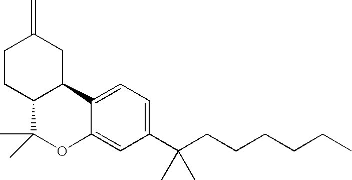 |
| JWH-102 & JWH-104 | 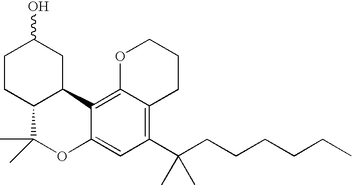 |
| O,2-Propano-9β-11-nor-HHC | 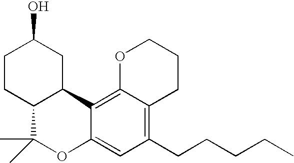 |
| JWH-056 | 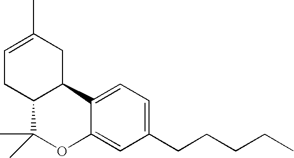 |
| JWH-132 | 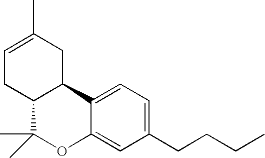 |
| JWH-125 | 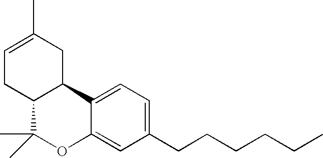 |
| JWH-075 | 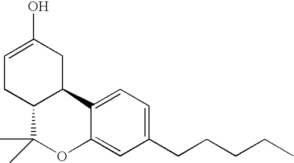 |

See, for example: Gareau et al., 1996; Huffman et al., 1999; Huffman, 2000; Pertwee et al., 2001; Goutopoulos et al., 2002; Khanolkar et al., 2001. Many more analogs are known in the art.

In one embodiment, the compound (e.g., the cannabinoid receptor inverse agonist or the cannabinoid receptor neutral antagonist) is selected from compounds of the following formula: 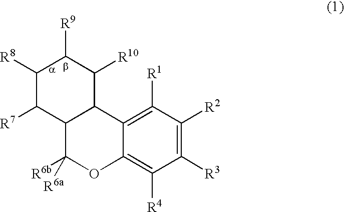
wherein:
α is a single bond, and β is a double bond; or
α is a double bond, and β is a single bond; or
α is a single bond, and β is a single bond;
R¹ is independently —H, —OH, an ether group, or an ester group;
R³ is independently C₁₋₁₂alkyl or substituted C₁₋₁₂alkyl;
each of R^{6a} and R^{6b} is independently —H, C₁₋₄alkyl, or substituted C₁₋₄alkyl;
R⁹ is independently —H, C₁₋₄alkyl, or substituted C₁₋₄alkyl;
when α is a single bond, and β is a single bond: then R⁹ may additionally be ═CH₂ or ═O;
each of R², R⁴, R⁷, R⁸, and R¹⁰ is independently-H, —OH, halo, C₁₋₄alkyl, or C₁₋₄alkoxy;
and stereoisomers thereof;
and pharmaceutically acceptable salts, solvates, amides, esters, ethers, chemically protected forms, and prodrugs thereof.

In one embodiment, α is a single bond, and β is a double bond.

In one embodiment, α is a double bond, and β is a single bond.

In one embodiment, α is a single bond, and β is a single bond.

In one embodiment, R¹ is independently —H, —OH, C₁₋₇alkoxy, C₁₋₇alkyl-acyloxy.

In one embodiment, R¹ is independently —H, —OH, —OMe, —OEt, —OC(═O)Me, —O(C═O)Et.

In one embodiment, R¹ is independently —H or —OH.

In one embodiment, R¹ is independently —OH.

In one embodiment, R¹ is independently —H.

In one embodiment, R³ is independently C₄₋₁₂alkyl or substituted C₄₋₁₂alkyl.

Examples of substituents include hydroxy, halo, azido, cyano, thioalkyl.

In one embodiment, R³ is independently C₄₋₁₂alkyl.

In one embodiment, R³ is independently linear or branched C₄₋₁₂alkyl.

In one embodiment, R³ is independently branched C₄₋₁₂alkyl.

In one embodiment, R³ is independently —C(CH₃)₂(CH₂)ₙCH₃, wherein n is 0, 1, 2, 3, 4, 5, 6, or 7.

In one embodiment, each of R^{6a} and R^{6b} is independently —H, C₁₋₄alkyl or hydroxy-C₁₋₄alkyl.

In one embodiment, each of R^{6a} and R^{6b} is independently C₁₋₄alkyl.

In one embodiment, each of R^{6a} and R^{6b} is independently -Me.

In one embodiment, R⁹ is independently —H, C₁₋₄alkyl, or hydroxy-C₁₋₄alkyl; or when α is a single bond, and β is a single bond: then R⁹ is —H, C₁₋₄alkyl, hydroxy-C₁₋₄alkyl, ═CH₂ or ═O.

In one embodiment, R⁹ is independently —H, Me, —CH₂OH; or when α is a single bond, and β is a single bond: then R⁹ is independently —H, Me, —CH₂OH, ═CH₂ or ═O.

In one embodiment, R⁹ is independently —H, -Me, or —CH₂OH.

In one embodiment, each of R², R⁴, R⁷, R⁸, and R¹⁰ is independently —H, —OH, -Me, or —OMe.

In one embodiment, each of R², R⁴, R⁷, R⁸, and R¹⁰ is independently —H or —OH.

In one embodiment, each of R², R⁴, R⁷, R⁸, and R¹⁰ is independently —H.

In one embodiment, the compound has a stereoisomeric structure corresponding to that of Δ⁹-THC.

For groups which are indicated to be “substituted” or “optionally substituted,” certain preferred substituents are described below under the heading “Some Preferred Substituents.”

In one embodiment, the compound is as structurally defined above, and additionally has one or more of the functional characteristics defined herein (e.g., has a particular cannabinoid receptor inhibition constant (Ki); is a cannabinoid receptor inverse agonist; is a cannabinoid receptor neutral antagonist; is a CB2 inverse agonist; etc.).

B. Non-Classical Cannabinoids

This class of ligands includes those which are structurally similar to Δ⁹-THC but which, for example, lack the oxygen atom of the 10aH-benzo[c]chromene core.

Thus, one aspect of the invention pertains to use of a compound (e.g., a cannabinoid receptor inverse agonist or a cannabinoid receptor neutral antagonist) for the manufacture of a medicament for the treatment of a bone disorder, wherein the compound is cannabidiol or an analogue or derivative thereof.

Another aspect of the present invention pertains to a method of treating a bone disorder comprising administering to a patient in need of treatment thereof a therapeutically effective amount of a compound (e.g., a cannabinoid receptor inverse agonist or a cannabinoid receptor neutral antagonist) that is cannabidiol or an analogue or derivative thereof.

Examples of such compounds include, but are not limited to, the following:

| Compound | Structure |
|---|---|
| cannabidol | 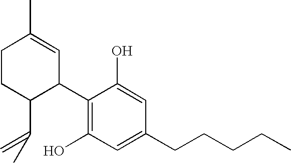 |
| HU-308 | 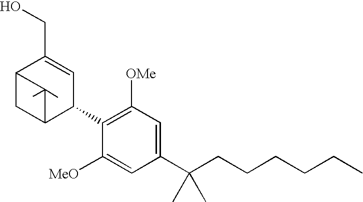 |
| CP-55940 | 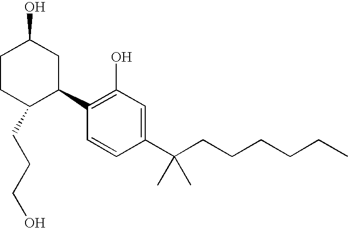 |
| CP-55244 | 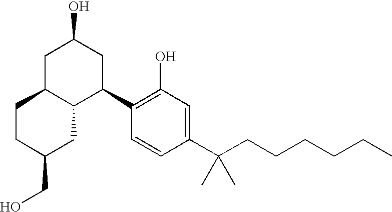 |
| O-1376 | 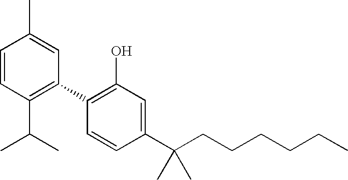 |
| O-1532 | 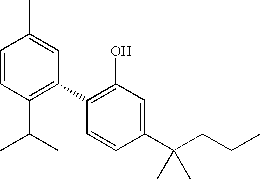 |
| O-1601 | 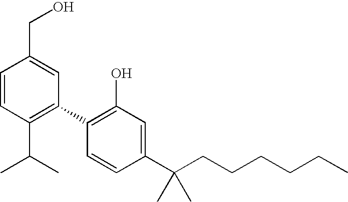 |
| O-1424; O-1422; O-1656; O-1160; O-1425; O-1661; O-1662; O-1423; O-1658; O-1659; O-1663; O-1657; O-1797A; O-1798B; O-1826; O-1871 | 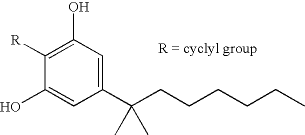 |
| O-1999; O-1964; O-1965; O-1962; O-2092; O-2122; O-2122; O-2114; O-2115; O-2123; O-2072; O-1966A; O-1967B; O-2121; O-2116A; O-2117B; O-2068; O-2090; O-2091 | 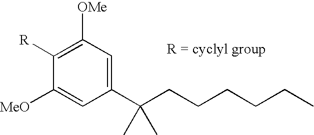 |
| O-1890 | 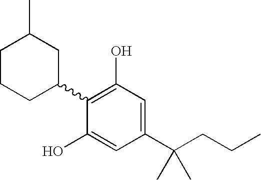 |
| CP-47497 | 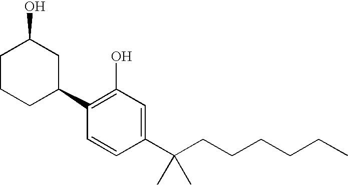 |

See, for example, Gareau et al., 1996; Huffman, 2000; Pertwee et al., 2001; Wiley et al., 2002; Goutopoulos et al., 2002. Many more analogs are known in the art.

In one embodiment, the compound (e.g., the cannabinoid receptor inverse agonist or the cannabinoid receptor neutral antagonist) is selected from compounds of the following formula: 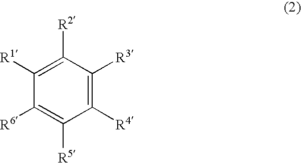
wherein:
R^{1′} is independently C₃₋₂₀carbocyclyl, C₃₋₂₀heterocyclyl, C₅₋₂₀aryl, and is optionally substituted;
R^{2′} is independently —H, —OH, or an ether group;
R^{4′} is independently C₁₋₁₂alkyl or substituted C₁₋₁₂alkyl;
R^{3′}, R^{5′}, and R^{6′} are independently —H, —OH, halo, C₁₋₄alkyl, or C₁₋₄alkoxy; and stereoisomers thereof;
and pharmaceutically acceptable salts, solvates, amides, esters, ethers, chemically protected forms, and prodrugs thereof.

In one embodiment, R^{1′} is independently C₃₋₂₀carbocyclyl; and is optionally substituted.

In one embodiment, R^{1′} is independently C₆₋₁₂carbocyclyl; and is optionally substituted.

In one embodiment, R^{1′} is independently:

cyclohexyl, cyclohexenyl, cyclohexadienyl, phenyl;

menthanyl, thujanyl;

caranyl, carenyl, caradienyl;

pinanyl, pinenyl, pinadienyl;

bornyl, bornenyl, bornadienyl;

tetralinyl, decalinyl;

or a saturated or unsaturated analogue or derivative thereof;

optionally substituted with one or more of —OH, C₁₋₄alkyl, and C₁₋₄alkoxy.

In one embodiment, R^{2′} is independently —H, —OH, or C₁₋₇alkoxy.

In one embodiment, R^{2′} is independently —H, —OH, —OMe, or —OEt.

In one embodiment, R^{2′} is independently —H or —OH.

In one embodiment, R^{2′} is independently —OH.

In one embodiment, R^{2′} is independently —H.

In one embodiment, R^{4′} is independently C₄₋₁₂alkyl or substituted C₄₋₁₂alkyl.

In one embodiment, R^{4′} is independently C₄₋₁₂alkyl.

In one embodiment, R^{4′} is independently linear or branched C₄₋₁₂alkyl.

In one embodiment, R^{4′} is independently branched C₄₋₁₂alkyl.

In one embodiment, R^{4′} is independently —C(CH₃)₂(CH₂)ₙCH₃, wherein n is 0, 1, 2, 3, 4, 5, 6, or 7.

In one embodiment, R^{4′} is independently —C(CH₃)₂(CH₂)ₙCH₃, wherein n is 3, 4, 5, 6, or 7.

In one embodiment, each of R^{3′}, R^{5′}, and R^{6′} is independently —H, —OH, -Me, or —OMe.

In one embodiment, each of R^{3′}, R^{5′}, and R^{6′} is independently —H or —OH.

In one embodiment, each of R^{3′}, R^{5′}, and R^{6′} is independently-H.

In one embodiment, R^{6′} is independently as defined for R^{2′}; and each of R^{3′} and R^{6′} is independently —H.

For groups which are indicated to be “substituted” or “optionally substituted,” certain preferred substituents are described below under the heading “Some Preferred Substituents.”

In one embodiment, the compound is as structurally defined above, and additionally has one or more of the functional characteristics defined herein (e.g., has a particular cannabinoid receptor inhibition constant (Ki); is a cannabinoid receptor inverse agonist is a cannabinoid receptor neutral antagonist; is a CB2 inverse agonist; etc.).

C. Aminoalkylindoles

This class of ligands includes those which have an indole core (which may be fused to another ring, e.g., as in WIN55212), often an N-substituted indole core, for example, an N-(amino-alkyl)-substituted indole core.

Thus, one aspect of the invention pertains to use of a compound (e.g., a cannabinoid receptor inverse agonist or a cannabinoid receptor neutral antagonist) for the manufacture of a medicament for the treatment of a bone disorder, wherein the compound has an indole core (e.g., is indole or an analogue or derivative thereof).

Another aspect of the present invention pertains to a method of treating a bone disorder comprising administering to a patient in need of treatment thereof a therapeutically effective amount of a compound (e.g., a cannabinoid receptor inverse agonist or a cannabinoid receptor neutral antagonist) having an indole core (e.g., is indole or an analogue or derivative thereof).

Examples of such compounds include, but are not limited to, the following:

| Compound | Structure |
|---|---|
| L768242 | 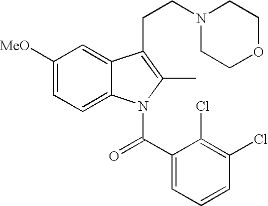 |
| BML-190 | 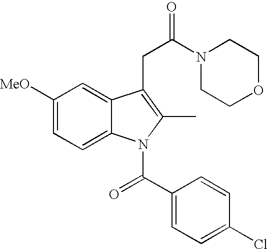 |
| AM-630 | 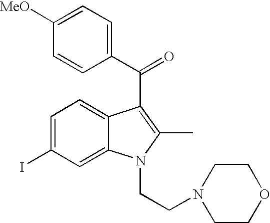 |
| AM-1241 | 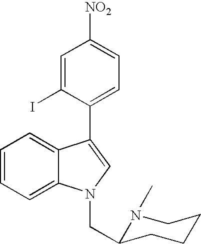 |
| L768242 | 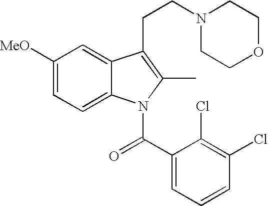 |
| AM-630 | 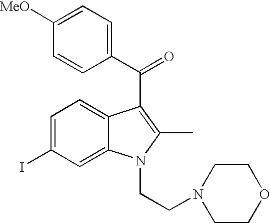 |
| AM-1241 | 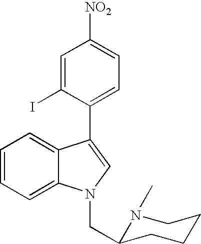 |
| JWH-015 | 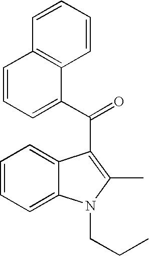 |
| JWH-018 | 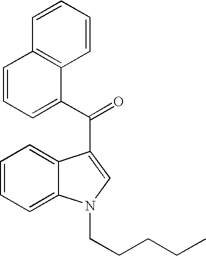 |
| JWH-070; JWH-071; JWH-072; JWH-073; JWH-019; JWH-020; JWH-071; JWH-016; JWH-007; JWH-004; JWH-009; JWH-077; JWH-078; JWH-079; JWH-080; JWH-081; JWH-082; JWH-083; JWH-094; JWH-096; JWH-098; JWH-099; JWH-100; JWH-046; JWH-047; JWH-048; JWH-049; JWH-050; JWH-076 | 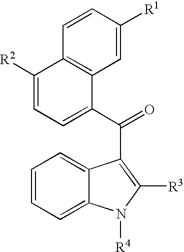 |
| Friary et al., 2003 | 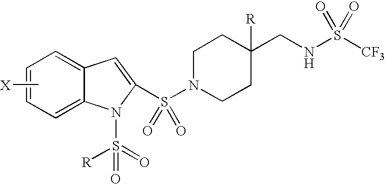 |
| WIN-55212-2 | 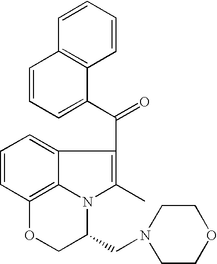 |

See, for example, Gallant et al., 1996; Melck et al., 2000; Huffman, 2000; Pertwee et al., 2001; Goutopoulos et al., 2002. Many more analogs are known in the art.

In one embodiment, the compound (e.g., the cannabinoid receptor inverse agonist or the cannabinoid receptor neutral antagonist) is selected from compounds of the following formula: 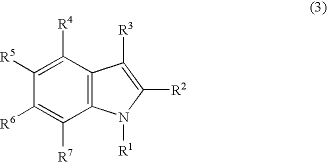
wherein:
R¹ is independently C₁₋₇alkyl, C₃₋₂₀heterocyclyl-C₁₋₇alkyl, C₅₋₂₀aryl-C₁₋₇alkyl, and is optionally substituted;
R² is independently —H, —OH, C₁₋₄alkyl, C₁₋₄alkoxy, or halo;
R³ is independently C₁₋₇alkyl, C₃₋₂₀heterocyclyl, C₅₋₂₀aryl, C₃₋₂₀heterocyclyl-C₁₋₇alkyl, C₅₋₂₀aryl-C₁₋₇alkyl, C₁₋₇alkyl-acyl, C₃₋₂₀heterocyclyl-acyl, C₅₋₂₀aryl-acyl, C₃₋₂₀heterocyclyl-C₁₋₇alkyl-acyl, or C₅₋₂₀aryl-C₁₋₇alkyl-acyl; and is optionally substituted;
each of R⁴ and R⁷ is independently —H, —OH, C₁₋₄alkyl, C₁₋₄alkoxy, or halo;
each of R⁵ and R⁶ is independently —H, —OH, C₁₋₄alkyl, C₁₋₄alkoxy, or halo; and stereoisomers thereof;
and pharmaceutically acceptable salts, solvates, amides, esters, ethers, chemically protected forms, and prodrugs thereof.

In one embodiment, R¹ is independently C₁₋₇alkyl, piperidinyl-C₁₋₇alkyl, morpholinyl-C₁₋₇alkyl, phenyl-C₁₋₇alkyl, and is optionally substituted.

In one embodiment, R² is independently —H or -Me.

In one embodiment, R² is independently —H.

In one embodiment, R³ is independently piperidinyl-C₁₋₇alkyl, morpholinyl-C₁₋₇alkyl, phenyl; phenyl-acyl; or naphthyl-acyl.

In one embodiment, each of R⁴ and R⁷ is independently —H, —OH, -Me, —OMe, or halo.

In one embodiment, each of R⁴ and R⁷ is independently —H.

In one embodiment, each of R⁵ and R⁶ is independently —H, —OH, -Me, —OMe, or halo.

In one embodiment, each of R⁵ and R⁶ is independently —H.

For groups which are indicated to be “substituted” or “optionally substituted,” certain preferred substituents are described below under the heading “Some Preferred Substituents.”

In one embodiment, the compound is as structurally defined above, and additionally has one or more of the functional characteristics defined herein (e.g., has a particular cannabinoid receptor inhibition constant (Ki); is a cannabinoid receptor inverse agonist is a cannabinoid receptor neutral antagonist; is a CB2 inverse agonist; etc.).

D. Endocannabinoids

This class of ligands includes those which are structurally similar to the endocannabinoid AEA.

Thus, one aspect of the invention pertains to use of a compound (e.g., a cannabinoid receptor inverse agonist or a cannabinoid receptor neutral antagonist) for the manufacture of a medicament for the treatment of a bone disorder, wherein the compound is AEA or an analogue or derivative thereof.

Another aspect of the present invention pertains to a method of treating a bone disorder comprising administering to a patient in need of treatment thereof a therapeutically effective amount of a compound (e.g., a cannabinoid receptor inverse agonist or a cannabinoid receptor neutral antagonist) that is AEA or an analogue or derivative thereof.

Examples of such compounds include, but are not limited to, the following:

| Compound | Structure |
|---|---|
| arachidonyl ethanolamide(AEA)(anandamide) | 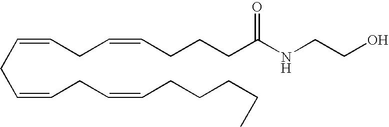 |
| homo-y-linolenylethanolamide | 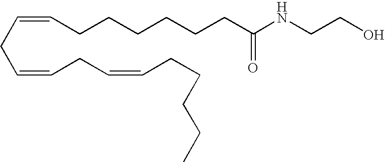 |
| 7,10,13,16- docosatetranylethyanolamide | 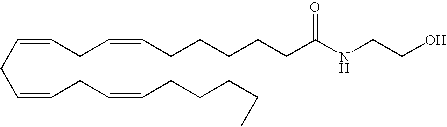 |
| 2-arachidonyl-glycerol(2AG) | 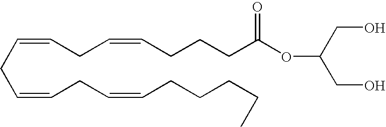 |
| R-(+)-methanandamide | 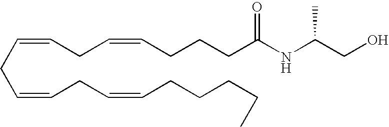 |
| AM-1116 | 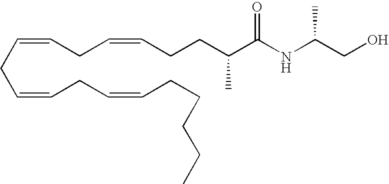 |
| O-1064 | 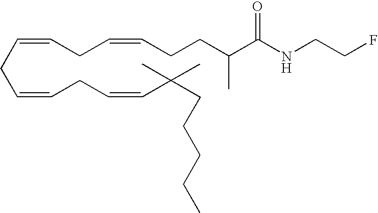 |
| ATFMK | 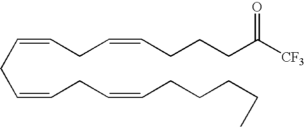 |
| AM-374 | 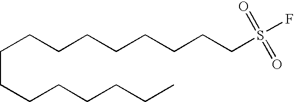 |
| AM-404 | 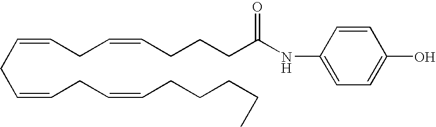 |
| arachidonylcyclopropylamide (ACPA) | 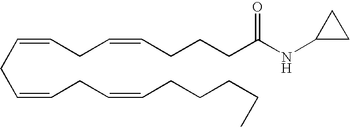 |
| arahcidonyl-2′-choroethylamide (ACEA) | 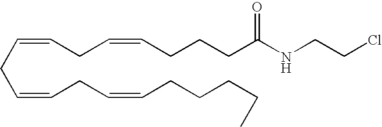 |
| Palmitoylethanolamide (PEA) | 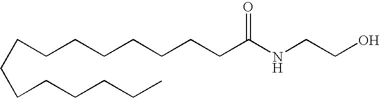 |
| Noladin ether | 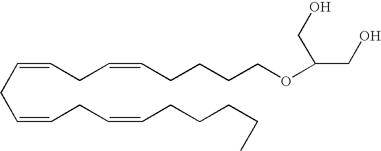 |
| N-[2-(3,4-Dihydroxyphenyl)ethyl]- 5Z,8Z,11Z,14Z-eicosatetraeneamide N-arachidonyldopamine(NADA) | 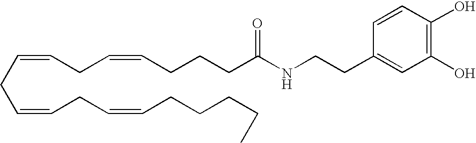 |

See, for example, Pertwee et al., 2001; Goutopoulos et al., 2002. Many more analogs are known in the art.

In one embodiment, the compound (e.g., the cannabinoid receptor inverse agonist or the cannabinoid receptor neutral antagonist) is selected from compounds of the following formula: 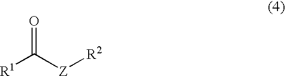
wherein:
Z is —NH— or —O—;
R¹ is independently linear or branched, saturated or partially unsaturated C₁₂₋₂₅alkyl; and is optionally substituted;
R² is independently C₁₋₄alkyl, C₃₋₂₀heterocyclyl, C₅₋₂₀aryl, C₃₋₂₀heterocyclyl-C₁₋₄alkyl, C₅₋₂₀aryl-C₁₋₄alkyl, hydroxy-C₁₋₄alkyl, hydroxy-C₃₋₂₀heterocyclyl, hydroxy-C₅₋₂₀aryl, hydroxy-C₃₋₂₀heterocyclyl-C₁₋₄alkyl, hydroxy-C₅₋₂₀aryl-C₁₋₄alkyl, halo-C₁₋₄alkyl, halo-C₃₋₂₀heterocyclyl, halo-C₅₋₂₀aryl, halo-C₃₋₂₀heterocyclyl-C₁₋₄alkyl, halo-C₅₋₂₀aryl-C₁₋₄alkyl; and
is optionally substituted;
and stereoisomers thereof;
and pharmaceutically acceptable salts, solvates, amides, esters, ethers, chemically protected forms, and prodrugs thereof.

In one embodiment, Z is independently —NH—.

In one embodiment, Z is independently —O—.

In one embodiment, R¹ is independently linear or branched, saturated or partially unsaturated C₁₈₋₂₅alkyl; and is optionally substituted.

In one embodiment, R¹ is independently linear or branched C₁₈₋₂₅alkyl having at least three carbon-carbon double bonds; and is optionally substituted.

In one embodiment, R¹ is independently linear or branched C₁₈₋₂₅alkyl having exactly four carbon-carbon double bonds; and is optionally substituted.

In one embodiment, R¹ is independently linear or branched C₁₈₋₂₅alkyl having a —(CH₂—CH═CH)₄— group; and is optionally substituted.

In one embodiment, R¹ is independently —(CH₂)₂—(CH₂—CH═CH)₄—(CH₂)₄—CH₃.

In one embodiment, R² is independently hydroxy-C₁₋₄alkyl, hydroxy-C₃₋₂₀heterocyclyl, hydroxy-C₅₋₂₀aryl, hydroxy-C₃₋₂₀heterocyclyl-C₁₋₄alkyl, hydroxy-C₅₋₂₀aryl-C₁₋₄alkyl; and is optionally substituted.

In one embodiment, R² is independently hydroxy-C₁₋₄alkyl, hydroxy-C₅₋₂₀aryl, hydroxy-C₅₋₂₀aryl-C₁₋₄alkyl; and is optionally substituted.

In one embodiment, R² is independently —CH₂CH₂OH, —CH(Me)CH₂OH, -Ph(OH), —CH₂CH₂(Ph(OH)₂).

For groups which are indicated to be “substituted” or “optionally substituted,” certain preferred substituents are described below under the heading “Some Preferred Substituents.”

In one embodiment, the compound is as structurally defined above, and additionally has one or more of the functional characteristics defined herein (e.g., has a particular cannabinoid receptor inhibition constant (Ki); is a cannabinoid receptor inverse agonist is a cannabinoid receptor neutral antagonist; is a CB2 inverse agonist; etc.).

E. Pyrazoles

This class of ligands includes those which have a pyrazole core, often a 1,5-disubstituted pyrazole core, for example, a 1,5-diaryl-pyrazole core, for example, a 1,5-diaryl-3-carboxamide pyrazole core.

Thus, one aspect of the invention pertains to use of a compound (e.g., a cannabinoid receptor inverse agonist or a cannabinoid receptor neutral antagonist) for the manufacture of a medicament for the treatment of a bone disorder, wherein the compound is pyrazole or an analogue or derivative thereof, e.g., has a pyrazole core, e.g., a 1,5-diaryl-pyrazole core, e.g., a 1,5-diaryl-3-carboxamide pyrazole core.

Another aspect of the present invention pertains to a method of treating a bone disorder comprising administering to a patient in need of treatment thereof a therapeutically effective amount of a compound (e.g., a cannabinoid receptor inverse agonist or a cannabinoid receptor neutral antagonist) having a pyrazole core (e.g., is pyrazole or an analogue or derivative thereof).

Examples of such compounds include, but are not limited to, the following:

| Compound | Structure |
|---|---|
| SR14176A | 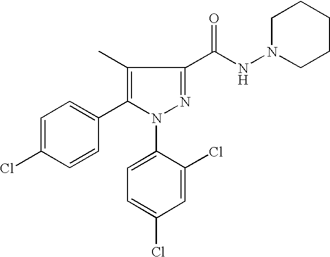 |
| AM-251 | 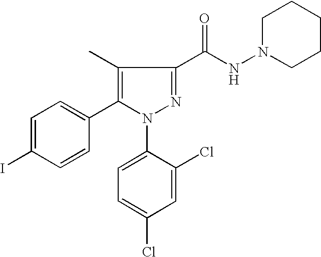 |
| AM-281 | 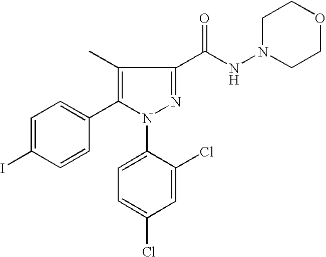 |
| SR144528 | 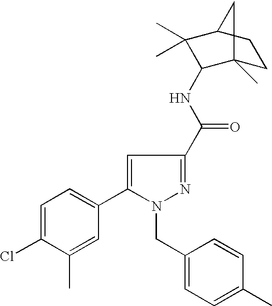 |
| Lan-99-07 | 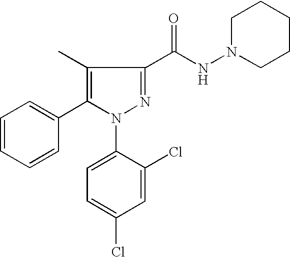 |
| Lan-99-08(para-NO₂); Lan-99-09(ortho-NO₂); Lan-99-10(para-NH₂); Lan-99-11(ortho-NH₂); Lan-99-12(para-I); Lan-99-13(ortho-I); | 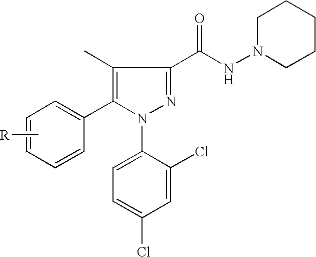 |
| Lan-99-16 | 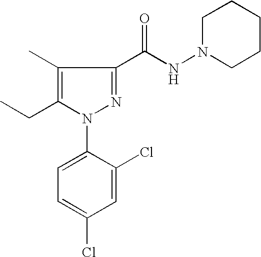 |
| Lan-99-19 | 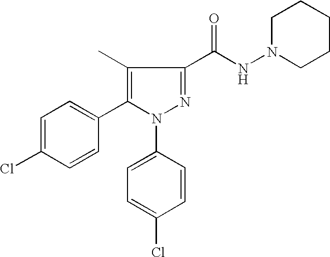 |
| Lan-99-22; Lan-99-23; Lan-99-24; Lan-99-25; Lan-99-26; Lan-99-27; Lan-99-28; Lan-99-29; Lan-99-30; Lan-99-31 | 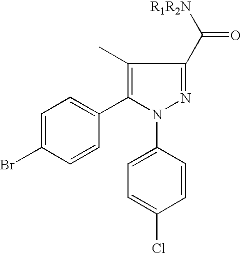 |
| O-848; O-849; O-850; O-852; O-853; O-869; O-870; O-889; O-890; O-909; O-910; O-1043;O-1269; O-1270; O-1271; O-1272; O-1398; O-1399; O-1876; O-1877 | 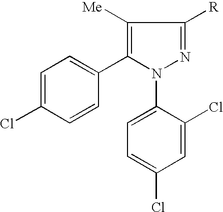 |
| O-1253; O-1254; O-1255; O-1300 | 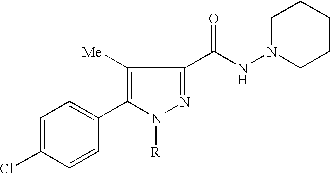 |
| O-1302; O-1559; O-1690; O-1691; O-1704; O-1710 | 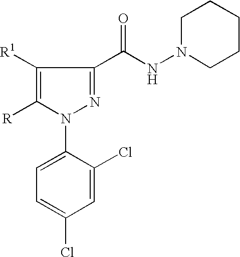 |
| O-1247; O-1248; O-1654; O-1655; O-1412; O-1343 | 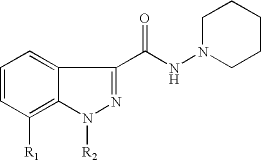 |
| (Makriyannis et al., 2003b) | 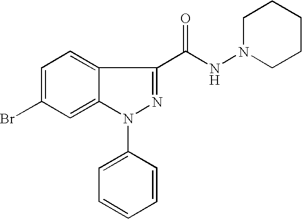 |
| Mussinu 1A-1R (Mussinu et al., 2003) | 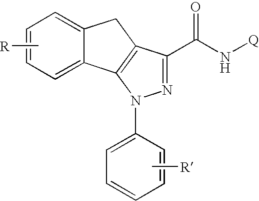 |
| (Krishnamurthy et al., 2004) | 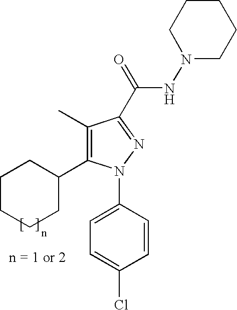 |
| NESS 0327 (Ruiu et al., 2003) | 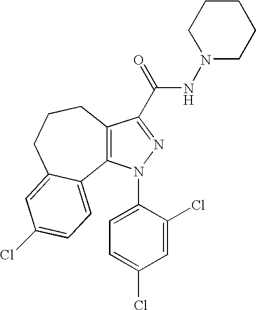 |
| O-1302 (Martin et al., 2003a) | 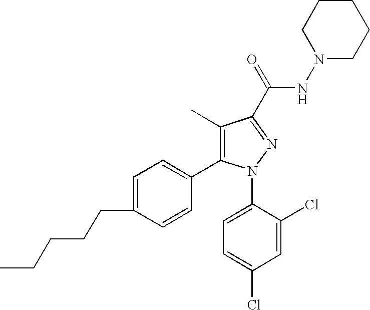 |
| (Makriyannis, A., et al., 2003a) | 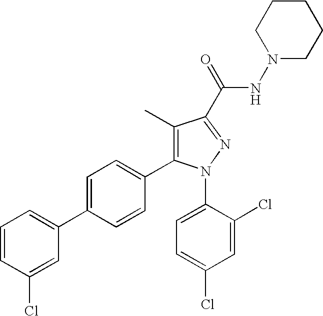 |
| (Makriyannis, A., et at, 2003a) | 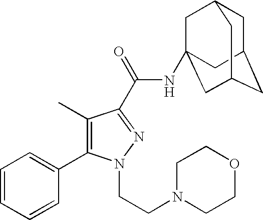 |
| (Makriyannis, A., et al., 2003a) | 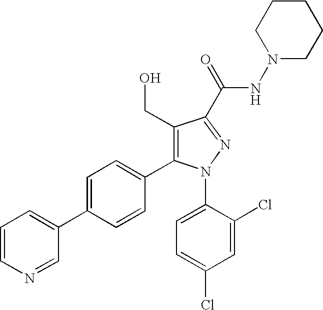 |
| (Makriyannis, A., et al., 2003a) | 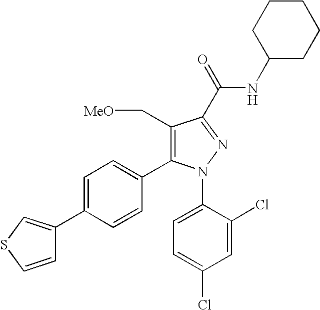 |

See, for example: Gatley et al., 1996; Lan et al., 1999; Pertwee et al., 2001; Wiley et al., 2001; Goutopoulos et al., 2002; Shim et al., 2002; Bass et al., 2002; Mussinu et al., 2003; Krishnamurthy et al., 2004; Ruiu et al., 2003; Martin et al., 2003a; Makriyannis, A., et al., 2003a. Many more analogs are known in the art.

In one embodiment, the compound is a pyrazole compound and is: celecoxib, deracoxib, or tepoxalin; or an analog or derivative thereof; or a pharmaceutically acceptable salt, solvate, amide, ester, ether, chemically protected form, or prodrug thereof.

Some Preferred Pyrazoles

In one embodiment, the compound (e.g., the cannabinoid receptor inverse agonist or the cannabinoid receptor neutral antagonist) is selected from compounds of the following formula: 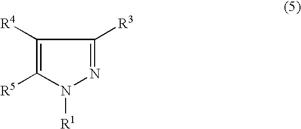
wherein:
R¹ is independently:

C₃₋₂₀carbocyclyl,

C₃₋₂₀heterocyclyl,

C₅₋₂₀aryl, or

C₅₋₂₀aryl-C₁₋₄alkyl;

and is optionally substituted;

R⁴ is independently:

—H,

C₁₋₄alkyl,

C₃₋₂₀heterocyclyl,

C₅₋₂₀aryl, or

C₅₋₂₀aryl-C₁₋₄alkyl;

and is optionally substituted;

R⁵ is independently:

C₁₋₄alkyl,

C₃₋₂₀carbocyclyl,

C₃₋₂₀heterocyclyl,

C₅₋₂₀aryl, or

C₅₋₂₀aryl-C₁₋₄alkyl;

and is optionally substituted;

R³ is independently:

amino-acyl;

amino-amino-acyl;

acyl;

acyl-oxy;

ether; or

and is optionally substituted;

and stereoisomers thereof;

and pharmaceutically acceptable salts, solvates, amides, esters, ethers, chemically protected forms, and prodrugs thereof.

Note the non-symmetry of the pyrazole ring, and the non-equivalence of the R³ and R⁴ substituents.

The Substituent R¹

In one embodiment, R¹ is independently C₅₋₂₀aryl or C₅₋₂₀aryl-C₁₋₄alkyl; and is optionally substituted.

In one embodiment, R¹ is independently phenyl or benzyl; and is optionally substituted.

In one embodiment, R¹ is independently C₅₋₂₀aryl; and is optionally substituted.

In one embodiment, R¹ is independently C₅₋₇aryl; and is optionally substituted.

In one embodiment, R¹ is independently C₆aryl; and is optionally substituted.

In one embodiment, R¹ is independently phenyl; and is optionally substituted.

In one embodiment, R¹ is independently selected from (“examples of optionally substituted phenyl groups”):

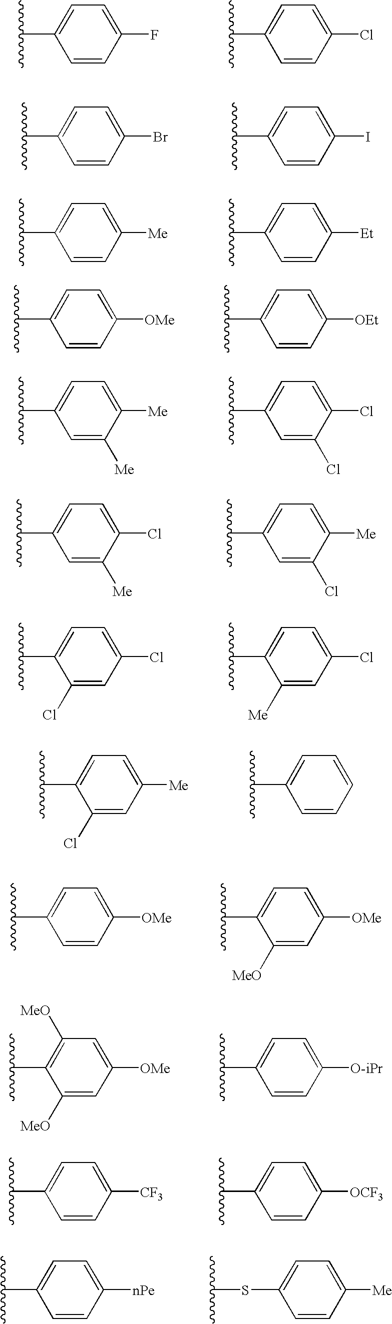

In one embodiment, R¹ is independently C₅₋₂₀aryl-C₁₋₄alkyl; and is optionally substituted.

In one embodiment, R¹ is independently C₅₋₇aryl-C₁₋₄alkyl; and is optionally substituted.

In one embodiment, R¹ is independently C₆aryl-C₁₋₄alkyl; and is optionally substituted.

In one embodiment, R¹ is independently benzyl; and is optionally substituted.

In one embodiment, R¹ is independently selected from (“examples of optionally substituted benzyl groups”):

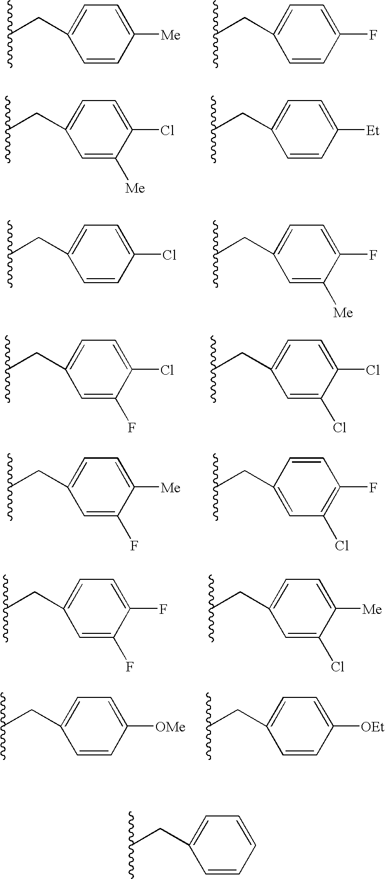

In one embodiment, R¹ is independently selected from: 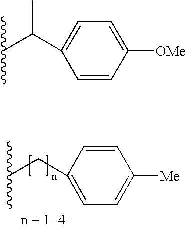
The Substituent R⁵

In one embodiment, R⁵ is independently:

C₃₋₂₀carbocyclyl,

C₃₋₂₀heterocyclyl,

C₅₋₂₀aryl, or

C₅₋₂₀aryl-C₁₋₄alkyl;

and is optionally substituted.

In one embodiment, R⁵ is independently C₅₋₂₀aryl or C₅₋₂₀aryl-C₁₋₄alkyl; and is optionally substituted.

In one embodiment, R⁵ is independently phenyl or benzyl; and is optionally substituted.

In one embodiment, R⁵ is independently C₅₋₂₀aryl; and is optionally substituted.

In one embodiment, R⁵ is independently C₅₋₇aryl; and is optionally substituted.

In one embodiment, R⁵ is independently C₆aryl; and is optionally substituted.

In one embodiment, R⁵ is independently phenyl; and is optionally substituted.

In one embodiment, R⁵ is independently selected from the “examples of optionally substituted phenyl groups” listed above for R¹.

In one embodiment, R⁵ is independently C₅₋₂₀aryl-C₁₋₄alkyl; and is optionally substituted.

In one embodiment, R⁵ is independently C₅₋₇aryl-C₁₋₄alkyl; and is optionally substituted.

In one embodiment, R⁵ is independently C₆aryl-C₁₋₄alkyl; and is optionally substituted.

In one embodiment, R⁵ is independently benzyl; and is optionally substituted.

In one embodiment, R⁵ is independently selected from the “examples of optionally substituted benzyl groups” listed above for R¹.

In one embodiment, R⁵ is independently selected from:

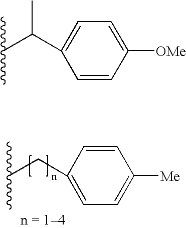

In one embodiment, R⁵ is independently C₁₋₄alkyl; and is optionally substituted.

In one embodiment, R⁵ is independently -Me, -Et, -nPr, -iPr, -nBu, -sBu, -iBu, -tBu, or -nPe.

All combinations of R¹ (as defined herein) and R⁵ (as defined herein) are explicitly disclosed herein. Some preferred combinations of R¹ and R⁵ are mentioned next.

In one embodiment, R¹ is independently C₅₋₂₀aryl-C₁₋₄alkyl, and is optionally substituted and R⁵ is independently C₅₋₂₀aryl, and is optionally substituted.

In one embodiment, R¹ is independently benzyl, and is optionally substituted and R⁵ is independently phenyl, and is optionally substituted.

In one embodiment, R¹ is independently C₅₋₂₀aryl, and is optionally substituted, and R⁵ is independently C₅₋₂₀aryl-C₁₋₄alkyl, and is optionally substituted.

In one embodiment, R¹ is independently phenyl, and is optionally substituted, and R⁵ is independently, benzyl, and is optionally substituted.

In one embodiment, R¹ is independently C₅₋₂₀aryl, and is optionally substituted and R⁵ is independently C₅₋₂₀aryl, and is optionally substituted.

In one embodiment, R¹ is independently phenyl, and is optionally substituted and R⁵ is independently phenyl, and is optionally substituted.

In one especially preferred embodiment, R¹ and R⁵ are as defined in the “combinations” immediately above, and R³ is independently: amino-acyl; or amino-amino-acyl; and is optionally substituted.

The Substituent R⁴

In one embodiment, R⁴ is independently C₁₋₄alkyl; and is optionally substituted (e.g., —CH₂—OMe, —CH₂—F, —CH₂—NHMe, etc.; see below).

In one embodiment, R⁴ is independently C₃₋₂₀heterocyclyl; and is optionally substituted.

In one embodiment, R⁴ is independently C₅₋₇aryl; and is optionally substituted.

In one embodiment, R⁴ is independently C₆aryl; and is optionally substituted.

In one embodiment, R⁴ is independently phenyl; and is optionally substituted.

In one embodiment, R⁴ is independently C₅₋₇aryl-C₁₋₄alkyl; and is optionally substituted.

In one embodiment, R⁴ is independently C₆aryl-C₁₋₄alkyl; and is optionally substituted.

In one embodiment, R⁴ is independently benzyl; and is optionally substituted.

In one embodiment, R⁴ is independently —H or C₁₋₄alkyl; and is optionally substituted.

In one embodiment, R⁴ is independently —H, -Me, -Et, -Ph, or —CH₂Ph.

In one embodiment, R⁴ is independently —H, -Me, or -Et.

In one embodiment, R⁴ is independently —H or -Me.

In one embodiment, R⁴ is independently -Me.

In one embodiment, R⁴ is independently —H.

In one embodiment, R⁴ and R⁵ together form a ring having from 5 to 7 ring atoms and fused to the parent pyrazole group; which ring is optionally substituted.

In one embodiment, R⁴ and R⁵ together form a phenyl ring fused to the parent pyrazole group; which phenyl ring is optionally substituted.

The Substituent R³-Amino-Acyl

In one embodiment, R³ is independently amino-acyl; and is optionally substituted.

In one embodiment, R³ is independently:

C₃₋₈cycloamino-acyl,

C₃₋₂₀carbocyclyl-amino-acyl,

C₃₋₂₀heterocyclyl-amino-acyl,

C₁₋₇alkyl-amino-acyl,

C₅₋₂₀aryl-amino-acyl,

C₅₋₂₀aryl-C₁₋₇alkyl-amino-acyl,

C₅₋₂₀heteroarylamino-acyl,

and is optionally substituted.

In one embodiment, R³ is independently C₃₋₈cycloamino-acyl; and is optionally substituted.

In one embodiment, R³ is independently piperidino-acyl, piperazino-acyl, morpholino-acyl, azepino-acyl; and is optionally substituted.

In one embodiment, R³ is independently selected from:

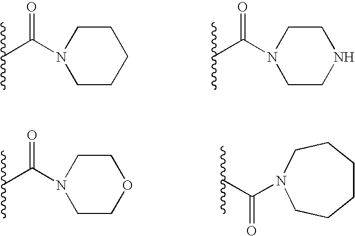

In one embodiment, R³ is independently C₃₋₂₀carbocyclyl-amino-acyl; and is optionally substituted.

In one embodiment, R³ is independently selected from:

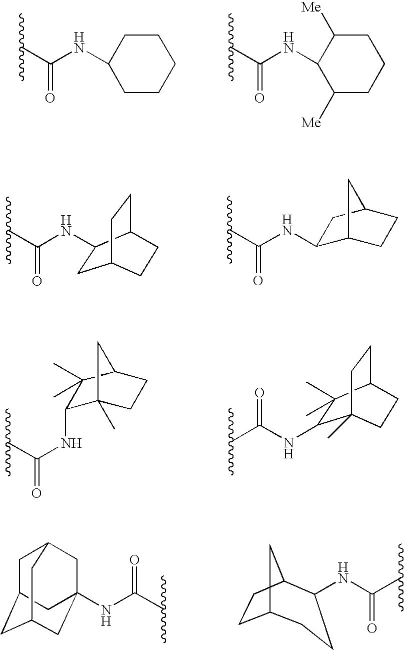

In one embodiment, R³ is independently C₁₋₇alkyl-amino-acyl; and is optionally substituted.

In one embodiment, R³ is independently selected from:

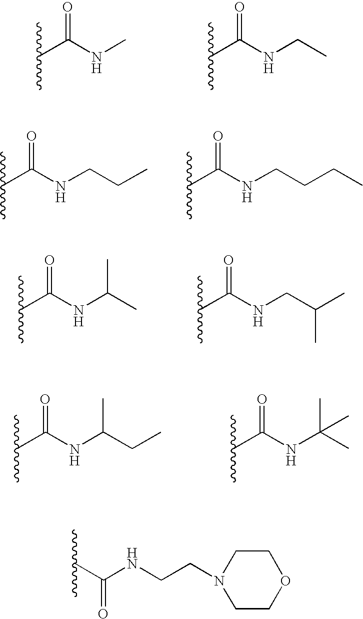

In one embodiment, R³ is independently C₅₋₂₀aryl-amino-acyl; and is optionally substituted.

In one embodiment, R³ is independently selected from:

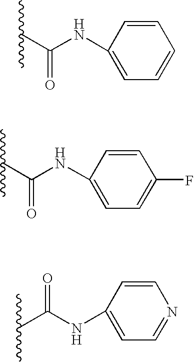

In one embodiment, R³ is independently phenyl-amino-acyl; and is optionally substituted.

In one embodiment, R³ is independently C₅₋₂₀aryl-C₁₋₇alkyl-amino-acyl; and is optionally substituted.

In one embodiment, R³ is independently selected from:

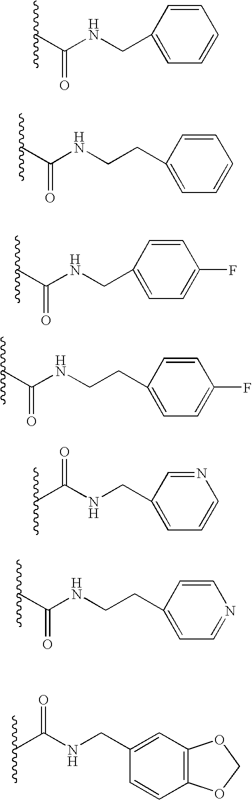

In one embodiment, R³ is independently benzyl-amino-acyl; and is optionally substituted.

In one embodiment, R³ is independently C₅₋₂₀heteroarylamino-acyl; and is optionally substituted. (A C₅₋₂₀heteroarylamino group is a C₅₋₂₀heteroaryl group having at least one aromatic nitrogen ring atom, and linked via that atom, e.g., as in pyrrolo.)

In one embodiment, R³ is independently pyrrolo-amino-acyl; and is optionally substituted.

In one embodiment, R³ is independently selected from: 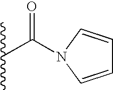
The Substituent R³-Amino-Amino-Acyl

In one embodiment, R³ is independently amino-amino-acyl; and is optionally substituted.

In one embodiment, R³ is independently:

C₃₋₈cycloamino-amino-acyl,

C₃₋₂₀carbocyclyl-amino-amino-acyl,

C₃₋₂₀heterocyclyl-amino-amino-acyl,

C₁₋₇alkyl-amino-amino-acyl,

C₅₋₂₀aryl-amino-amino-acyl,

C₅₋₂₀aryl-C₁₋₇alkyl-amino-amino-acyl,

C₅₋₂₀heterarylamino-amino-acyl,

and is optionally-substituted.

In one embodiment, R³ is independently C₃₋₈cycloamino-amino-acyl; and is optionally substituted.

In one embodiment, R³ is independently piperidino-amino-acyl, piperazino-amino-acyl, morpholino-amino-acyl, or azepino-amino-acyl; and is optionally substituted.

In one embodiment, R³ is independently phenyl-amino-amino-acyl, benzyl-amino-amino-acyl, or pyrrolo-amino-acyl; and is optionally substituted.

In one embodiment, R³ is independently selected from: 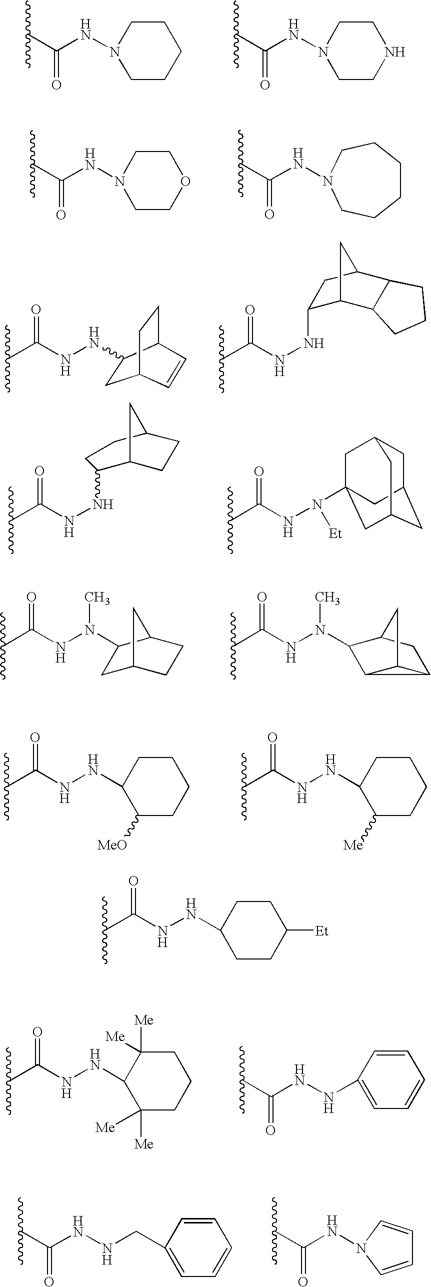
The Substituent R³-Acyl

In one embodiment, R³ is independently acyl; and is optionally substituted.

In one embodiment, R³ is independently:

C₁₋₇alkyl-acyl,

C₅₋₂₀aryl-acyl, or

C₅₋₂₀aryl-C₁₋₇alkyl-acyl;-

and is optionally substituted.

In one embodiment, R³ is independently:

C₅₋₂₀aryl-acyl, or

C₅₋₂₀aryl-C₁₋₇alkyl-acyl,

and is optionally substituted.

In one embodiment, R³ is independently C₅₋₂₀aryl-acyl, and is optionally substituted.

In one embodiment, R³ is independently phenyl-acyl, and is optionally substituted.

In one embodiment, R³ is independently benzyl-acyl, and is optionally substituted.

In one embodiment, R³ is independently selected from: 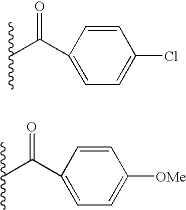
The Substituent R³-Acyl-Oxy

In one embodiment, R³ is independently acyl-oxy; and is optionally substituted.

In one embodiment, R³ is independently:

C₁₋₇alkyl-acyl-oxy,

C₅₋₂₀aryl-acyl-oxy, or

C₅₋₂₀aryl-C₁₋₇alkyl-acyl-oxy;

and is optionally substituted.

In one embodiment, R³ is independently:

C₅₋₂₀aryl-acyl-oxy, or

C₅₋₂₀aryl-C₁₋₇alkyl-acyl-oxy,

and is optionally substituted.

In one embodiment, R³ is independently C₅₋₂₀aryl-acyl-oxy, and is optionally substituted.

In one embodiment, R³ is independently phenyl-acyl-oxy, and is optionally substituted.

In one embodiment, R³ is independently benzyl-acyl-oxy, and is optionally substituted.

In one embodiment, R³ is independently selected from: 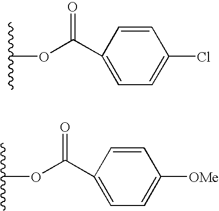
The Substituent R³-Ether

In one embodiment, R³ is independently ether; and is optionally substituted.

In one embodiment, R³ is independently:

C₁₋₇alkyl-oxy (C₁₋₇alkoxy),

C₅₋₂₀aryl-oxy, or

C₅₋₂₀aryl-C₁₋₇alkyl-oxy;

and is optionally substituted.

In one embodiment, R³ is independently C₁₋₇alkoxy; and is optionally substituted.

In one embodiment, R³ is independently selected from: —O-Me, —O-Et, —O-nPr, —O-iPr, —O-nBu, —O-sBu, —O-iBu, —O-tBu, and —O-nPe.

For groups which are indicated to be “substituted” or “optionally substituted,” certain preferred substituents are described below under the heading “Some Preferred Substituents.”

In one embodiment, the compound is as structurally defined above, and additionally has one or more of the functional characteristics defined herein (e.g., has a particular cannabinoid receptor inhibition constant (Ki); is a cannabinoid receptor inverse agonist is a cannabinoid receptor neutral antagonist; is a CB2 inverse agonist; etc.).

Many pyrazole compounds have been described as possible treatments for osteoporosis and other bone diseases. See, for example: Anantanarayan et al., 1999; Anantanarayan et al., 2002; Anantanarayan et al., 2003a; Anantanarayan et al., 2003b; Blake et al., 1997; Didierlaurent et al., 1999; Dragovich et al., 2003; Green et al., 2003; Hale et al., 2003; Hanson et al., 2002; Hanson et al., 2003; Hanson et al., 2000; Huebner et al., 2001; Kapadia et al., 2002; Kolasa et al., 2002; Weier et al., 1999; Weier et al., 2003. However, the presently claimed pyrazole compounds are structurally distinct from the pyrazole compounds previously associated with osteoporosis, and furthermore, the pyrazole compounds known for treatment of bone diseases are not known to be CB receptor inverse agonists or neutral antagonists.

F. Miscellaneous Compounds

This class of ligands includes those which do not fall within the previous classes. Examples of such compounds include, but are not limited to, the following:

| Compound | Structure |
|---|---|
| CP-50556(L-nantradol) | 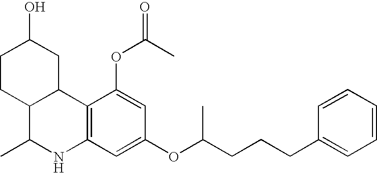 |
| LY320135 (see, e.g., Felder et al., 1998) | 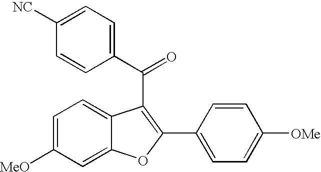 |
| Pyridone-5 & Pyridone-21 (see, e.g., Huffman et al., 2001) | 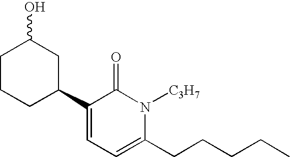 |
| JTE-907 (see, e.g., Iwamura et al., 2001; Inaba et al., 2001) | 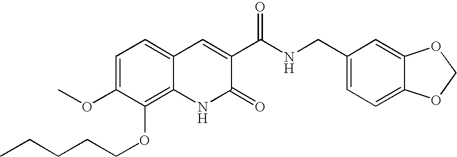 |
| Kozlowski et al., 2003 | 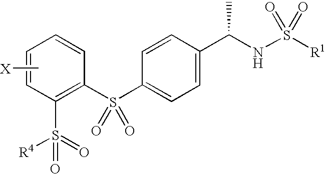 |
| Kruse et al., 2003b | 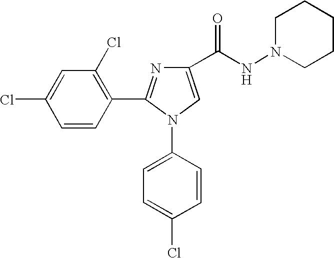 |
| Kruse et al., 2003a | 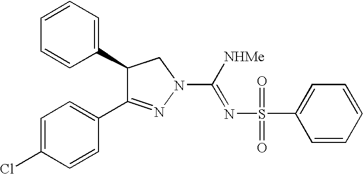 |
| Finke et al., 2003 | 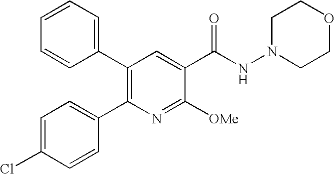 |
| Hagmann et al., 2003a | 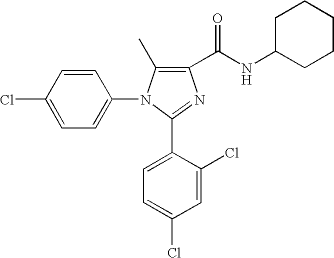 |
| Khanolkar et al., 2001 | 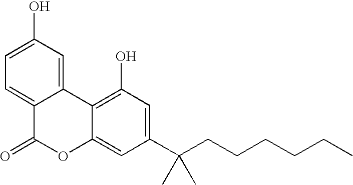 |
| Martin et al., 2003b (O-1797) | 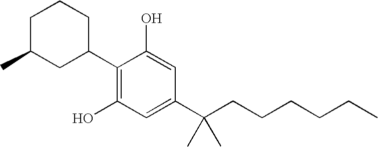 |
| Barth et al., 2003a | 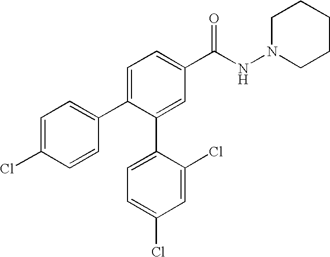 |
| Hagmann et al., 2003b | 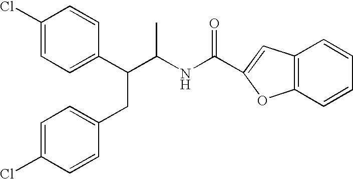 |
| Lange et al., 2003 | 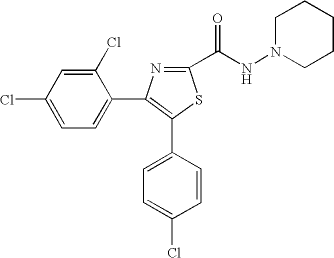 |
| Garzon et al., 2003 | 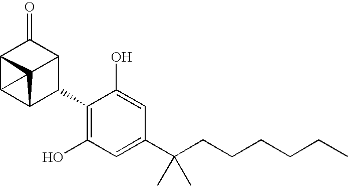 |
| Barth et al., 2003b | 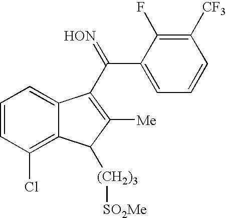 |

Thus, one aspect of the invention pertains to use of a compound for the manufacture of a medicament for the treatment of a bone disorder, wherein the compound has a 2-oxoquinoline core (e.g., is 2-oxoquinoline or an analogue or derivative thereof.

Another aspect of the present invention pertains to a method of treating a bone disorder comprising administering to a patient in need of treatment thereof a therapeutically effective amount of compound (e.g., a cannabinoid receptor inverse agonist or a cannabinoid receptor neutral antagonist) having a 2-oxoquinoline core (e.g., is 2-oxoquinoline or an analogue or derivative thereof).

In one embodiment (see, e.g., Inaba et al., 2001; Iwamura et al., 2001), the compound (e.g., the cannabinoid receptor inverse agonist or the cannabinoid receptor neutral antagonist) is selected from compounds (2-oxoquinolines) of the following formula: 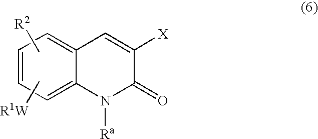
wherein:
W is —O—, —S(O)ᵣ—, —CR³R⁴—, —NR⁵—, —NR⁵CO—, —CONR⁵—, —COO—, or —OCO— (where R³ and R⁴ may be identical or different and are hydrogen atom or C₁₋₇alkyl;
R⁵ is hydrogen atom or C₁₋₇alkyl; and t is 0, 1 or 2);
R¹ represents hydrogen atom, C₁₋₇alkyl, C₁₋₇alkenyl, C₁₋₇alkynyl, C₅₋₂₀aryl, C₅₋₂₀aryl-C₁₋₇alkyl, C₅₋₂₀heteroaryl, C₅₋₂₀heteroaryl-C₁₋₇alkyl, C₁₋₇cycloalkyl, or C₁₋₇cycloalkyl-C₁₋₇alkyl;
each group of R¹, except hydrogen atom, may be substituted or unsubstituted with C₁₋₇alkyl-amino, amino, hydroxy, C₁₋₇alkoxy, carboxy, C₁₋₇alkoxycarbonyl, acyl, acyloxy, acylthio, mercapto, C₁₋₇alkyl-thio, C₁₋₇alkyl-sulfinyl or C₁₋₇alkyl-sulfonyl;
each group, except hydrogen atom and C₁₋₇alkyl, may be substituted or unsubstituted with C₁₋₇alkyl;
R² represents hydrogen atom, C₁₋₇alkyl, —OR⁶
(where R⁶ represents hydrogen atom, C₁₋₇alkyl, C₁₋₇alkenyl, C₁₋₇alkynyl, C₅₋₂₀aryl, C₅₋₂₀aryl-C₁₋₇alkyl, C₅₋₂₀heteroaryl, C₅₋₂₀heteroaryl-C₁₋₇alkyl, C₁₋₇cycloalkyl or C₁₋₇cycloalkyl-C₁₋₇alkyl),
—NR⁷R⁸
(where R⁷ and R⁸ may be identical or different and are hydrogen atom, C₁₋₇alkyl, C₁₋₇alkenyl, C₁₋₇alkynyl, acyl, C₅₋₂₀aryl, C₅₋₂₀aryl-C₁₋₇alkyl, C₅₋₂₀heteroaryl, C₅₋₂₀heteroaryl-C₁₋₇alkyl, C₁₋₇cycloalkyl or C₁₋₇cycloalkyl-C₁₋₇alkyl; or R⁷ and R⁸, together with the adjacent nitrogen atom, can form a C₅₋₂₀heteroaryl); or
—(CH₂)ᵤ—S(O)ᵤR⁹
(where R⁹ represents hydrogen atom, C₁₋₇alkyl, C₁₋₇alkenyl or C₁₋₇alkynyl, each of u and u′ independently is 0, 1 or 2);
each group of R², except hydrogen atom, may be substituted or unsubstituted with C₁₋₇alkyl-amino, amino, hydroxy, C₁₋₇alkoxy, C₁₋₇alkoxycarbonyl, acyl, acyloxy, acylthio, mercapto, C₁₋₇alkylthio, C₁₋₇alkyl-sulfinyl or C₁₋₇alkyl-sulfonyl;
each group, except hydrogen atom and C₁₋₇alkyl, may be substituted or unsubstituted with C₁₋₇alkyl;
R^{a} represents hydrogen atom or C₁₋₇alkyl;
X represents —COOR^{b}, —CONH₂, —CONR^{c}-(Alk^{a})ᵣ-R, —(CH₂)ₚ—OC(═Y)—NR^{d}-(Alk^{b})ₛ-R, —(CH₂)_{q}—NR^{e}—C(=Z)-(NR^{f})_{w}(Alk^{c})ᵥ-R, —(CH₂)ₚ—OH or —(CH₂)—NR^{e}R^{e′}
(where each of R^{b}, R^{c}, R^{d}, and R^{f} independently represents hydrogen atom or C₁₋₇alkyl;
each of R^{e} and R^{e′} independently represents hydrogen atom or C₁₋₇alkyl;
or R^{e} and R^{e′} together with the adjacent nitrogen atom, can form a C₅₋₂₀heteroaryl;
each of Alk^{a}, Alk^{b} and Alk^{c} independently represents C₁₋₇alkylene or C₁₋₇alkenylene;
each of the C₁₋₇alkylene and C₁₋₇alkenylene may be substituted or unsubstituted with hydroxy, carboxy, C₁₋₇alkoxycarbonyl, C₁₋₇alkyl (the C₁₋₇alkyl may be substituted or unsubstituted with hydroxy, C₁₋₇alkoxy or C₁₋₇alkyl-thio) or —CONR¹⁰R¹¹ (where R¹⁰ and R¹¹ may be identical or different and are hydrogen atom or C₁₋₇alkyl; or R¹⁰ and R¹¹, together with the adjacent nitrogen atom, can form a C₅₋₂₀heteroaryl);
R represents C₅₋₂₀aryl, C₅₋₂₀heteroaryl, C₁₋₇cycloalkyl, benzene-condensed C₁₋₇cycloalkyl or 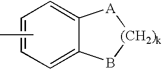
where A and B independently represent oxygen atom, nitrogen atom or sulfur atom; k is an integer of 1-3;
each of the C₅₋₂₀aryl and C₅₋₂₀heteroaryl may be substituted or unsubstituted with a C₁₋₇alkyl substituted or unsubstituted with hydroxy or may be substituted or unsubstituted with a hydroxy, C₁₋₇alkoxy, C₁₋₇alkenyloxy, acyl, acyloxy, halogen atom, nitro, amino, sulfonamide, C₁₋₇alkylamino, C₅₋₂₀aryl-C₁₋₇alkyloxy, pyridyl, piperidino, carboxy, C₁₋₇alkoxycarbonyl, acylamino, aminocarbonyl, cyano or glucuronic acid residue;
the C₁₋₇cycloalkyl may be substituted or unsubstituted with a hydroxy, C₁₋₇alkoxy or ═O;
the benzene-condensed C₁₋₇cycloalkyl may be substituted or unsubstituted with a hydroxy or C₁₋₇alkoxy;
each of r, s, v and w independently is 0 or 1;
each of Y and Z independently represents a nitrogen atom, oxygen atom or sulfur atom; and
each of p and q independently represents an integer of 1-4;
and stereoisomers thereof;
and pharmaceutically acceptable salts, solvates, amides, esters, ethers, chemically protected forms, and prodrugs thereof.

For groups which are indicated to be “substituted” or “optionally substituted,” certain preferred substituents are described below under the heading “Some Preferred Substituents.”

In one embodiment, the compound is as structurally defined above, and additionally has one or more of the functional characteristics defined herein (e.g., has a particular cannabinoid receptor inhibition constant (Ki); is a cannabinoid receptor inverse agonist; is a cannabinoid receptor neutral antagonist; is a CB2 inverse agonist; etc.).

Some Preferred Substituents

In many of the embodiments discussed above, chemical moieties are referred to as “substituted” or “optionally substituted,” that is, (optionally) bearing one or more substituents. In one embodiment, each of those substituents, if present, is independently selected from:

(1) carboxylic acid; (2) ester; (3) amido; (4) acyl; (5) halo; (6) cyano; (7) nitro; (8) hydroxy; (9) ether; (10) thiol; (11) thioether; (12) acyloxy; (13) amino; (14) acylamino; (15) aminoacylamino; (16) sulfonamino; (17) sulfonyl; (18) sulfonate; (19) sulfonamido; (20) C₅₋₂₀aryl-C₁₋₇alkyl; (21) C₅₋₂₀aryl; (22) C₃₋₂₀heterocyclyl; (23) C₁₋₇alkyl; (24) oxo; (25) imino; (26) hydroxyimino.

In one embodiment, each of those substituents is independently selected from:
(1) —C(═O)OH;
(2) —C(═O)OR¹, wherein R¹ is independently as defined in (20), (21), (22) or (23);
(3) —C(═O)NR²R³, wherein each of R² and R³ is independently —H; or as defined in (20), (21), (22) or (23); or R² and R³ taken together with the nitrogen atom to which they are attached form a ring having from 3 to 7 ring atoms;
(4) —C(═O)R⁴, wherein R⁴ is independently as defined in (20), (21), (22) or (23);
(5) —F, —Cl, —Br, —I;
(6) —CN;
(7) —NO₂;
(8) —OH;
(9) —OR⁵, wherein R⁵ is independently as defined in (20), (21), (22) or (23);
(10) —SH;
(11) —SR⁶, wherein R⁵ is independently as defined in (20), (21), (22) or (23);
(12) —OC(═O)R⁷, wherein R⁷ is independently as defined in (20), (21), (22) or (23);
(13) —NR⁸R⁹, wherein each of R⁸ and R⁹ is independently —H; or as defined in (20), (21), (22) or (23); or R⁸ and R⁹ taken together with the nitrogen atom to which they are attached form a ring having from 3 to 7 ring atoms;
(14) —NR¹⁰C(═O)R¹¹, wherein R¹⁰ is independently-H; or as defined in (20), (21), (22) or (23); and R¹¹ is independently —H, or as defined in (20), (21), (22) or (23);
(15) —NR¹²C(═O)NR¹³R¹⁴ or —NR¹²C(═S)NR¹³R¹⁴, wherein R¹² is independently —H; or as defined in (20), (21), (22) or (23); and each of R¹³ and R¹⁴ is independently —H; or as defined in (20), (21), (22) or (23); or R¹³ and R¹⁴ taken together with the nitrogen atom to which they are attached form a ring having from 3 to 7 ring atoms;
(16) —NR¹⁵SO₂R¹⁶, wherein R¹⁵ is independently —H; or as defined in (20), (21), (22) or (23); and R¹⁶ is independently —H, or as defined in (20), (21), (22) or (23);
(17) —SO₂R¹⁷, wherein R¹⁷ is independently as defined in (20), (21), (22) or (23);
(18) —OSO₂R¹⁸, wherein R¹⁸ is independently as defined in (20), (21), (22) or (23);
(19) —SO₂NR¹⁹R²⁰, wherein each of R¹⁹ and R²⁰ is independently —H; or as defined in (20), (21), (22) or (23); or R¹⁹ and R²⁰ taken together with the nitrogen atom to which they are attached form a ring having from 3 to 7 ring atoms;
(20) C₅₋₂₀aryl-C₁₋₇alkyl, for example, wherein C₅₋₂₀aryl is as defined in (21); unsubstituted or substituted with one or more groups as defined in (1) to (26);
(21) C₅₋₂₀aryl, including C₆₋₂₀carboaryl and C₅₋₂₀heteroaryl; unsubstituted or substituted with one or more groups as defined in (1) to (26);
(22) C₃₋₂₀heterocyclyl; unsubstituted or substituted with one or more groups as defined in (1) to (26);
(23) C₁₋₇alkyl, including:
   unsaturated C₁₋₇alkyl, e.g., C₂₋₇alkenyl and C₂₋₇alkynyl;
   cyclic C₁₋₇alkyl, e.g., C₃₋₇cycloalkyl C₃₋₇cycloalkenyl, C₃₋₇cycloalkynyl;
   C₁₋₇alkyl substituted with one or more groups as defined in (1) to (22) and (24) to (26),
      e.g., halo-C₁₋₇alkyl;
      e.g., amino-C₁₋₇alkyl (e.g., —(CH₂)_{w}-amino, w is 1, 2, 3, or 4);
      e.g., carboxy-C₁₋₇alkyl (e.g., —(CH₂)_{w}—COOH, w is 1, 2, 3, or 4);
      e.g., hydroxy-C₁₋₇alkyl (e.g., —(CH₂)ₙ—OH, w is 1, 2, 3, or 4);
      e.g., C₁₋₇alkoxy-C₁₋₇alkyl (e.g., —(CH₂)_{w}—O—C₁₋₇alkyl, w is 1, 2, 3, or 4);
(24) ═O;
(25) ═NR²¹, wherein R²¹ is independently —H; or as defined in (20), (21), (22) or (23);
(26) ═NOH.

In one embodiment, each of those substituents is independently selected from:
(1) —C(═O)OH;
(2) —C(═O)OMe, —C(═O)OEt, —C(═O)O(iPr), —C(═O)O(tBu); —C(═O)O(cPr); —C(═O)OCH₂CH₂OH, —C(═O)OCH₂CH₂OMe, —C(—O)OCH₂CH₂OEt; —C(═O)OPh, —C(═O)OCH₂Ph;
(3) —(C═O)NH₂, —(C═O)NMe₂, —(C═O)NEt₂, —(C═O)N(iPr)₂, —(C═O)N(CH₂CH₂OH)₂; —(C═O)-morpholino, —(C═O)NHPh, —(C═O)NHCH₂Ph;
(4) —(C═O)Me, —(C═O)Et, —(C═O)(tBu), —(C═O)-cHex, —(C═O)Ph; —(C═O)CH₂Ph;
(5) —F, —Cl, —Br, —I;
(6) —CN;
(7) —NO₂;
(8) —OH;
(9) —OMe, —OEt, —O(iPr), —O(tBu), —OPh, —OCH₂Ph;
   —OCF₃, —OCH₂CF₃;
   —OCH₂CH₂OH, —OCH₂CH₂OMe, —OCH₂CH₂OEt;
   —OCH₂CH₂NH₂, —OCH₂CH₂NMe₂, —OCH₂CH₂N(iPr)₂;
   —OPh-Me, —OPh-OH, —OPh-OMe, —OPh-F, —OPh-Cl, —OPh-Br, —OPh-I;
(10) —SH;
(11) —SMe, —SEt, —SPh, —SCH₂Ph;
(12) —OC(═O)Me, —OC(═O)Et, —OC(═O)(iPr), —OC(═O)(tBu); —OC(═O)(cPr); —OC(═O)CH₂CH₂OH, —OC(═O)CH₂CH₂OMe, —OC(═O)CH₂CH₂OEt; —OC(═O)Ph, —OC(═O)CH₂Ph;
(13) —NH₂, —NHMe, —NHEt, —NH(iPr), —NMe₂, —NEt₂, —N(iPr)₂, —N(CH₂CH₂OH)₂; —NHPh, —NHCH₂Ph; piperidino, piperazino, morpholino;
(14) —NH(C═O)Me, —NH(C═O)Et, —NH(C═O)Ph, —NHC(═O)CH₂Ph; —NMe(C═O)Me, —NMe(C═O)Et, —NMe(C═O)Ph, —NMeC(═O)CH₂Ph;
(15) —NH(C═O)NH₂, —NH(C═O)NHMe, —NH(C═O)NHEt, —NH(C═O)NPh, —NH(C═O)NHCH₂Ph; —NH(C═S)NH₂, —NH(C═S)NHMe, —NH(C═S)NHEt, —NH(C═S)NPh, —NH(C═S)NHCH₂Ph;
(16) —NHSO₂Me, —NHSO₂Et, —NHSO₂Ph, —NHSO₂PhMe, —NHSO₂CH₂Ph; —NMeSO₂Me, —NMeSO₂Et, —NMeSO₂Ph, —NMeSO₂PhMe, —NMeSO₂CH₂Ph;
(17) —SO₂Me, —SO₂CF₃, —SO₂Et, —SO₂Ph, —SO₂PhMe, —SO₂CH₂Ph;
(18) —OSO₂Me, —OSO₂CF₃, —OSO₂Et, —OSO₂Ph, —OSO₂PhMe, —OSO₂CH₂Ph;
(19) —SO₂NH₂, —SO₂NHMe, —SO₂NHEt, —SO₂NMe₂, —SO₂NEt₂, —SO₂-morpholino, —SO₂NHPh, —SO₂NHCH₂Ph;
(20) —CH₂Ph, —CH₂Ph-Me, —CH₂Ph-OH, —CH₂Ph-F, —CH₂Ph-Cl;
(21) -Ph, -Ph-Me, -Ph-OH, -Ph-OMe, -Ph-F, -Ph-Cl, -Ph-Br, -Ph-I;
   pyridyl, pyrazinyl, pyrimidinyl, pyridazinyl; furanyl, thiophenyl, pyrrolyl, imidazolyl, pyrazolyl, oxazolyl, thiazolyl;
(22) pyrrolidinyl, piperidinyl, azepinyl, tetrahydropyranyl, morpholinyl, azetidinyl, piperazinyl, imidazolinyl, piperazinedionyl, and oxazolinonyl;
(23)-Me, -Et, -nPr, -iPr, -nBu, -iBu, -sBu, -tBu, -nPe;
   -cPr, -cHex, —CH═CH₂, —CH₂—CH═CH₂;
   —CF₃, —CHF₂, —CH₂F, —CCl₃, —CBr₃, —CH₂CH₂F, —CH₂CHF₂, and —CH₂CF₃;
   —CH₂OH, —CH₂OMe, —CH₂OEt, —CH₂NH₂, —CH₂NMe₂;
   —CH₂CH₂OH, —CH₂CH₂OMe, —CH₂CH₂OEt, —CH₂CH₂CH₂NH₂, —CH₂CH₂NMe₂;
(24) ═O;
(25) ═NH, ═NMe; ═NEt;
(26) ═NOH.

In one embodiment, each of those substituents is independently selected from:

(2) —C(═O)OMe;

(3) —(C═O)NH₂, —(C═O)NMe₂;

(4) —(C═O)Me, —(C═O)Et;

(5) —F, —Cl, —Br, —I;

(6) —CN;

(7) —NO₂;

(8) —OH;

(9) —OMe, —OEt;

(10) —SH;

(11) —SMe;

(13) —NH₂, —NMe₂, —NEt₂;

(21) -Ph;

(23) -Me, -Et, —CF₃.

Some Preferred Examples

In one embodiment, the compound is selected from: AM251; AM630; SR144528; “methyl”; “piperidyl”; “benzodioxo”; SR141716A; O-1184; JTE-907; AM281; cannabinol; cannabidiol; and Δ⁹-THC.

In one embodiment, the compound is selected from: AM251; AM630; SR144528; SR141716A; O-1184; JTE-907; AM281; cannabinol; cannabidiol; and Δ⁹-THC.

In one embodiment, the compound is selected from: AM251; AM630; SR144528; “methyl”; “piperidyl”; “benzodioxo”; SR141716A.

In one embodiment, the compound is selected from: SR141716A and AM251.

Chemical Terms

The term “carbo,” “carbyl,” “hydrocarbon” and “hydrocarbyl,” as used herein, pertain to compounds and/or groups which have only carbon and hydrogen atoms (but see “carbocyclic” below).

The term “hetero,” as used herein, pertains to compounds and/or groups which have at least one heteroatom, for example, multivalent heteroatoms (which are also suitable as ring heteroatoms) such as boron, silicon, nitrogen, phosphorus, oxygen, sulfur, and selenium (more commonly nitrogen, oxygen, and sulfur) and monovalent heteroatoms, such as fluorine, chlorine, bromine, and iodine.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “ring,” as used herein, pertains to a closed ring of from 3 to 10 covalently linked atoms, more preferably 3 to 8 covalently linked atoms, yet more preferably 5 to 6 covalently linked atoms. A ring may be an alicyclic ring or an aromatic ring. The term “alicyclic ring,” as used herein, pertains to a ring which is not an aromatic ring.

The term “carbocyclic ring,” as used herein, pertains to a ring wherein all of the ring atoms are carbon atoms.

The term “carboaromatic ring,” as used herein, pertains to an aromatic ring wherein all of the ring atoms are carbon atoms.

The term “heterocyclic ring,” as used herein, pertains to a ring wherein at least one of the ring atoms is a multivalent ring heteroatom, for example, nitrogen, phosphorus, silicon, oxygen, or sulfur, though more commonly nitrogen, oxygen, or sulfur. Preferably, the heterocyclic ring has from 1 to 4 heteroatoms.

The term “cyclic compound,” as used herein, pertains to a compound which has at least one ring. The term “cyclyl,” as used herein, pertains to a monovalent moiety obtained by removing a hydrogen atom from a ring atom of a cyclic compound.

Where a cyclic compound has two or more rings, they may be fused (e.g., as in naphthalene), bridged (e.g., as in norbornane), spiro (e.g., as in spiro[3.3]heptane), or a combination thereof. Cyclic compounds with one ring may be referred to as “monocyclic” or “mononuclear,” whereas cyclic compounds with two or more rings may be referred to as “polycyclic” or “polynuclear.”

The term “carbocyclic compound,” as used herein, pertains to a cyclic compound which has only carbocyclic ring(s).

The term “heterocyclic compound,” as used herein, pertains to a cyclic compound which has at least one heterocyclic ring.

The term “aromatic compound,” as used herein, pertains to a cyclic compound which has at least one aromatic ring.

The term “carboaromatic compound,” as used herein, pertains to a cyclic compound which has only carboaromatic ring(s).

The term “heteroaromatic compound,” as used herein, pertains to a cyclic compound which has at least one heteroaromatic ring.

The term “monodentate substituents,” as used herein, pertains to substituents which have one point of covalent attachment.

The term “monovalent monodentate substituents,” as used herein, pertains to substituents which have one point of covalent attachment, via a single bond. Examples of such substituents include halo, hydroxy, and alkyl.

The term “multivalent monodentate substituents,” as used herein, pertains to substituents which have one point of covalent attachment, but through a double bond or triple bond. Examples of such substituents include oxo, imino, alkylidene, and alklidyne.

The term “bidentate substituents,” as used herein, pertains to substituents which have two points of covalent attachment, and which act as a linking group between two other moieties. Examples of such substituents include alkylene and arylene.

Substituents

The phrase “optionally substituted,” as used herein, pertains to a parent group which may be unsubstituted or which may be substituted.

Unless otherwise specified, the term “substituted,” as used herein, pertains to a parent group which bears one or more substituents. The term “substituent” is used herein in the conventional sense and refers to a chemical moiety which is covalently attached to, appended to, or if appropriate, fused to, a parent group. A wide variety of substituents are well known, and methods for their formation and introduction into a variety of parent groups are also well known.

The substituents are described in more detail below.

Alkyl: The term “alkyl,” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated, partially unsaturated, or fully unsaturated. Thus, the term “alkyl” includes the sub-classes alkenyl, alkynyl, cycloalkyl, etc., discussed below.

In this context, the prefixes (e.g., C₁₋₄, C₁₋₇, C₁₋₂₀, C₂₋₇, C₃₋₇, etc.) denote the number of carbon atoms, or range of number of carbon atoms. For example, the term “C₁₋₄alkyl,” as used herein, pertains to an alkyl group having from 1 to 4 carbon atoms. Examples of groups of alkyl groups include C₁₋₄alkyl (“lower alkyl”), C₁₋₇alkyl, and C₁₋₂₀alkyl.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Cycloalkyl: The term “cycloalkyl,” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 20 ring atoms (unless otherwise specified). Preferably, each ring has from 3 to 7 ring atoms.

Examples of (unsubstituted) saturated cylcoalkyl groups include, but are not limited to, those derived from: cyclopropane (C₃), cyclobutane (C₄), cyclopentane (C₅), cyclohexane (C₆), cycloheptane (C₇), norbornane (C₇), norpinane (C₇), norcarane (C₇), adamantane (C₁₀), and decalin (decahydronaphthalene) (C₁₀).

Examples of (substituted) saturated cycloalkyl groups, which are also referred to herein as “alkyl-cycloalkyl” groups, include, but are not limited to, methylcyclopropyl, dimethylcyclopropyl, methylcyclobutyl, dimethylcyclobutyl, methylcyclopentyl, dimethylcyclopentyl, methylcyclohexyl, and dimethylcyclohexyl, menthane, thujane, carane, pinane, bornane, norcarane, and camphene.

Examples of (substituted) unsaturated cyclic alkenyl groups, which are also referred to herein as “alkyl-cycloalkenyl” groups, include, but are not limited to, methylcyclopropenyl, dimethylcyclopropenyl, methylcyclobutenyl, dimethylcyclobutenyl, methylcyclopentenyl, dimethylcyclopentenyl, methylcyclohexenyl, and dimethylcyclohexenyl.

Examples of (substituted) cycloalkyl groups, with one or more other rings fused to the parent cycloalkyl group, include, but are not limited to, those derived from: indene (C₉), indan (e.g., 2,3-dihydro-1H-indene) (C₉), tetraline (1,2,3,4-tetrahydronaphthalene (C₁₀), acenaphthene (C₁₂), fluorene (C₁₃), phenalene (C₁₃), acephenanthrene (C₁₅), aceanthrene (C₁₆). For example, 2H-inden-2-yl is a C₅cycloalkyl group with a substituent (phenyl) fused thereto.

Alkenyl: The term “alkenyl,” as used herein, pertains to an alkyl group having one or more carbon-carbon double bonds. Examples of groups of alkenyl groups include C₂₋₄alkenyl, C₂₋₇alkenyl, C₂₋₂₀alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (—C(CH₃)═CH₂), butenyl (C₄), pentenyl (C₅), and hexenyl (C₆).

Examples of (unsubstituted) unsaturated cyclic alkenyl groups, which are also referred to herein as “cycloalkenyl” groups, include, but are net limited to, cyclopropenyl (C₃), cyclobutenyl (C₄), cyclopentenyl (C₅), and cyclohexenyl (C₆).

Alkynyl: The term “alkynyl,” as used herein, pertains to an alkyl group having one or more carbon-carbon triple bonds. Examples of groups of alkynyl groups include C₂₋₄alkynyl, C₂₋₇alkynyl, C₂₋₂₀alkynyl.

Examples of (unsubstituted) unsaturated alkynyl groups include, but are not limited to, ethynyl (ethinyl, —C≡CH) and 2-propynyl (propargyl, —CH₂—C≡CH).

Alkylidene: The term “alkylidene,” as used herein, pertains to a divalent monodentate moiety obtained by removing two hydrogen atoms from a carbon atom of a hydrocarbon compound having from 1 to 20 carbon atoms (unless otherwise specified), which may be aliphatic or alicyclic, or a combination thereof, and which may be saturated, partially unsaturated, or fully unsaturated. Examples of groups of alkylidene groups include C₁₋₄alkylidene, C₁₋₇alkylidene, C₁₋₂₀alkylidene.

Examples of alkylidene groups include, but are not limited to, methylidene (═CH₂), ethylidene (═CH—CH₃), vinylidene (═C═CH₂), and isopropylidene (═C(CH₃)₂). An example of a substituted alkylidene is benzylidene (═CH-Ph).

Alkylidyne: The term “alkylidyne,” as used herein, pertains to a trivalent monodentate moiety obtained by removing three hydrogen atoms from a carbon atom of a hydrocarbon compound having from 1 to 20 carbon atoms (unless otherwise specified), which may be aliphatic or alicyclic, or a combination thereof, and which may be saturated, partially unsaturated, or fully unsaturated. Examples of groups of alkylidyne groups include C₁₋₄alkylidyne, C₁₋₇alkylidyne, C₁₋₂₀alkylidyne.

Examples of alkylidyne groups include, but are not limited to, methylidyne (≡CH) and ethylidyne (≡C—CH₃).

Carbocyclyl: The term “carbocyclyl,” as used herein, pertains to a monovalent moiety obtained by removing a hydrogen atom from a ring atom of a carbocyclic compound, which moiety has from 3 to 20 ring atoms (unless otherwise specified). Preferably, each ring has from 3 to 7 ring atoms.

In this context, the prefixes (e.g., C₃₋₂₀, C₃₋₇, C₅₋₆, etc.) denote the number of ring atoms, or range of number of ring atoms. For example, the term “C₅₋₆carbocyclyl,” as used herein, pertains to a carbocyclyl group having 5 or 6 ring atoms. Examples of groups of carbocyclyl groups include C₃₋₂₀carbocyclyl, C₃₋₁₀carbocyclyl, C₅₋₁₀carbocyclyl, C₃₋₇carbocyclyl, and C₅₋₇carbocyclyl.

Examples of carbocyclic groups include, but are not limited to, those described above as cycloalkyl groups; and those described below as carboaryl groups.

Heterocyclyl: The term “heterocyclyl,” as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆heterocyclyl,” as used herein, pertains to a heterocyclyl group having 5 or 6 ring atoms. Examples of groups of heterocyclyl groups include C₃₋₂₀heterocyclyl, C₃₋₇heterocyclyl, C₅₋₇heterocyclyl, and C₅₋₆heterocyclyl.

Examples of (non-aromatic) monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of heterocyclyl groups which are also heteroaryl groups are described below with aryl groups.

Aryl: The term “aryl,” as used herein, pertains to a monovalent moiety obtained by removing a hydrogen atom from an aromatic ring atom of an aromatic compound, which moiety has from 3 to 20 ring atoms (unless otherwise specified). Preferably, each ring has from 5 to 7 ring atoms.

In this context, the prefixes (e.g., C₃₋₂₀, C₅₋₇, C₅₋₆, etc.) denote the number of ring atoms, or range of number of ring atoms, whether carbon atoms or heteroatoms. For example, the term “C₅₋₆aryl,” as used herein, pertains to an aryl group having 5 or 6 ring atoms. Examples of groups of aryl groups include C₃₋₂₀aryl, C₃₋₁₂aryl, C₅₋₁₂aryl, C₅₋₇aryl, and C₅₋₆aryl.

The ring atoms may be all carbon atoms, as in “carboaryl groups” (e.g., C₅₋₂₀carboaryl).

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include but are not limited to, groups derived from indene (C₉), isoindene (C₉), and fluorene (C₁₃).

Alternatively, the ring atoms may include one or more heteroatoms, as in “heteroaryl groups” (e.g., C₅₋₂₀heteroaryl).

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heterocyclic groups (some of which are also heteroaryl groups) which comprise fused rings, include, but are not limited to:

C₉heterocyclic groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀heterocyclic groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₃heterocyclic groups (with 3 fused rings) derived from carbazole (N₁), dibenzofuran (O₁), dibenzothiophene (S₁), carboline (N₂), perimidine (N₂), pyridoindole (N₂); and,

C₁₄heterocyclic groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Heterocyclic groups (including heteroaryl groups) which have a nitrogen ring atom in the form of an —NH— group may be N-substituted, that is, as —NR—. For example, pyrrole may be N-methyl substituted, to give N-methypyrrole. Examples of N-substituents include, but are not limited to C₁₋₇alkyl, C₃₋₂₀heterocyclyl, C₅₋₂₀aryl, and acyl groups.

Heterocyclic groups (including heteroaryl groups) which have a nitrogen ring atom in the form of an —N═ group may be substituted in the form of an N-oxide, that is, as —N(→O)=(also denoted —N⁺(→O⁻)=). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms. Monocyclic examples of such groups include, but are not limited to, those derived from:

C₅: cyclopentanone, cyclopentenone, cyclopentadienone;

C₆: cyclohexanone, cyclohexenone, cyclohexadienone;

O₁: furanone (C₅), pyrone (C₆);

N₁: pyrrolidone (pyrrolidinone) (C₅), piperidinone (piperidone) (C₆), piperidinedione (C₆);

N₂: imidazolidone (imidazolidinone) (C₅), pyrazolone (pyrazolinone) (C₅), piperazinone (C₆), piperazinedione (C₅), pyridazinone (C₆), pyrimidinone (C₆) (e.g., cytosine), pyrimidinedione (C₆) (e.g., thymine, uracil), barbituric acid (C₆);

N₁S₁: thiazolone (C₅), isothiazolone (C₅);

N₁O₁: oxazolinone (C₅).

Polycyclic examples of such groups include, but are not limited to, those derived from:

C₉: indenedione;

C₁₀: tetralone, decalone;

C₁₄: anthrone, phenanthrone;

N₁: oxindole (C₉);

O₁: benzopyrone (e.g., coumarin, isocoumarin, chromone) (C₁₀);

N₁O₁: benzoxazolinone (C₉), benzoxazolinone (C₁₀);

N₂: quinazolinedione (C₁₀);

N₄: purinone (C₉) (e.g., guanine).

Still more examples of cyclic groups which bear one or more oxo (═O) groups on ring carbon atoms include, but are not limited to, those derived from:

cyclic anhydrides (—C(═O)—O—C(═O)— in a ring), including but not limited to maleic anhydride (C₅), succinic anhydride (C₅), and glutaric anhydride (C₆);

cyclic carbonates (—O—C(═O)—O— in a ring), such as ethylene carbonate (C₅) and 1,2-propylene carbonate (C₅);

imides (—C(═O)—NR—C(═O)— in a ring), including but not limited to, succinimide (C₅), maleimide (C₅), phthalimide, and glutarimide (C₆);

lactones (cyclic esters, —O—C(═O)— in a ring), including, but not limited to, β-propiolactone, γ-butyrolactone, δ-valerolactone (2-piperidone), and ε-caprolactone;

lactams (cyclic amides, —NR—C(═O)— in a ring), including, but not limited to, β-propiolactam (C₄), γ-butyrolactam (2-pyrrolidone) (C₅), δ-valerolactam (C₆), and ε-caprolactam (C₇);

cyclic carbamates (—O—C(═O)—NR— in a ring), such as 2-oxazolidone (C₅);

cyclic ureas (—NR—C(═O)—NR— in a ring), such as 2-imidazolidone (C₅) and pyrimidine-2,4-dione (e.g., thymine, uracil) (C₆).

The above alkyl, alkylidene, alkylidyne, heterocyclyl, and aryl groups, whether alone or part of another substituent, may themselves optionally be substituted with one or more groups selected from themselves and the additional substituents listed below.

Hydrogen: —H. Note that if the substituent at a particular position is hydrogen, it may be convenient to refer to the compound as being “unsubstituted” at that position.

Halo: —F, —Cl, —Br, and —I.

Hydroxy: —OH.

Ether: —OR, wherein R is an ether substituent, for example, a C₁₋₇alkyl group (also referred to as a C₁₋₇alkoxy group, discussed below), a C₃₋₂₀heterocyclyl group (also referred to as a C₃₋₂₀heterocyclyloxy group), or a C₅₋₂₀aryl group (also referred to as a C₅₋₂₀aryloxy group), preferably a C₁₋₇alkyl group.

C₁₋₇alkoxy: —OR, wherein R is a C₁₋₇alkyl group. Examples of C₁₋₇alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇alkyl group, a C₃₋₂₀heterocyclyl group, or a C₅₋₂₀aryl group, preferably a C₁₋₇alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇alkyl group, a C₃₋₂₀heterocyclyl group, or a C₅₋₂₀aryl group, preferably a C₁₋₇alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇alkyl group, a C₃₋₂₀heterocyclyl group, or a C₅₋₂₀aryl group, preferably a C₁₋₇alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇alkyl group, a C₃₋₂₀heterocyclyl group, or a C₅₋₂₀aryl group, preferably a C₁₋₇alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇alkyl group (also referred to as C₁₋₇alkylacyl or C₁₋₇alkanoyl), a C₃₋₂₀heterocyclyl group (also referred to as C₃₋₂₀heterocyclylacyl), or a C₅₋₂₀aryl group (also referred to as C₅₋₂₀arylacyl), preferably a C₁₋₇alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Acylhalide (haloformyl, halocarbonyl): —C(═O)X, wherein X is —F, —Cl, —Br, or —I, preferably —Cl, —Br, or —I.

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇alkyl group, a C₃₋₂₀heterocyclyl group, or a C₅₋₂₀aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —OC(═O)OCH₃, —OC(═O)OCH₂CH₃, —OC(═O)OC(CH₃)₃, and —OC(═O)OPh.

Amido (carbamoyl, carbamyl, aminocarbonyl, carboxamide): —C(═O)NR R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido (acylamino): —NR¹C(═O)R², wherein R¹ is an amide substituent, for example, hydrogen, a C₁₋₇alkyl group, a C₃₋₂₀heterocyclyl group, or a C₅₋₂₀aryl group, preferably hydrogen or a C₁₋₇alkyl group, and R² is an acyl substituent, for example, a C₁₋₇alkyl group, a C₃₋₂₀heterocyclyl group, or a C₅₋₂₀aryl group, preferably hydrogen or a C₁₋₇alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

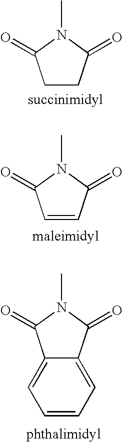

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Ureido: —N(R¹)CONR²R³ wherein R² and R³ are independently amino substituents, as defined for amino groups, and R1 is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

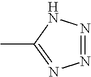

Amino: —NR¹R², wherein R¹ and R² are independently amino substituents, for example, hydrogen, a C₁₋₇alkyl group (also referred to as C₁₋₇alkylamino or di-C₁₋₇alkylamino), a C₃₋₂₀heterocyclyl group, or a C₅₋₂₀aryl group, preferably H or a C₁₋₇alkyl group, or, in the case of a “cyclic” amino group (“cycloamino”), R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Imino: ═NR, wherein R is an imino substituent, for example, for example, hydrogen, a C₁₋₇alkyl group, a C₃₋₂₀heterocyclyl group, or a C₅₋₂₀aryl group, preferably H or a C₁₋₇alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇alkyl group, a C₃₋₂₀heterocyclyl group, or a C₅₋₂₀aryl group, preferably H or a C₁₋₇alkyl group. Examples of amidine groups include, but are not limited to, —C(═NH)NH₂, —C(═N H)NMe₂, and —C(═NMe)NMe₂.

Nitro: —NO₂.

Cyano (nitrile, carbonitrile): —CN.

Isocyano: —NC.

Cyanato: —OCN.

Isocyanato: —NCO.

Isothiocyano (isothiocyanato): —NCS.

Sulfhydryl (thiol, mercapto): —SH.

Thioether (sulfide): —SR, wherein R is a thioether substituent, for example, a C₁₋₇alkyl group (also referred to as a C₁₋₇alkylthio group), a C₃₋₂₀heterocyclyl group, or a C₅₋₂₀aryl group, preferably a C₁₋₇alkyl group. Examples of C₁₋₇alkylthio groups include, but are not limited to, —SCH₃ and —SCH₂CH₃.

Disulfide: —SS—R, wherein R is a disulfide substituent, for example, a C₁₋₇alkyl group, a C₃₋₂₀heterocyclyl group, or a C₅₋₂₀aryl group, preferably a C₁₋₇alkyl group (also referred to herein as C₁₋₇alkyl disulfide). Examples of C₁₋₇alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇alkyl group, a C₃₋₂₀heterocyclyl group, or a C₅₋₂₀aryl group, preferably a C₁₋₇alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇alkyl group, a C₃₋₂₀heterocyclyl group, or a C₅₋₂₀aryl group, preferably a C₁₋₇alkyl group, including, for example, a fluorinated or perfluorinated C₁₋₇alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇alkyl group, a C₃₋₂₀heterocyclyl group, or a C₅₋₂₀aryl group, preferably a C₁₋₇alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇alkyl group, a C₃₋₂₀heterocyclyl group, or a C₅₋₂₀aryl group, preferably a C₁₋₇alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇alkyl group, a C₃₋₂₀heterocyclyl group, or a C₅₋₂₀aryl group, preferably a C₁₋₇alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇alkyl group, a C₃₋₂₀heterocyclyl group, or a C₅₋₂₀aryl group, preferably a C₁₋₇alkyl group.

Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇alkyl group, a C₃₋₂₀heterocyclyl group, or a C₅₋₂₀aryl group, preferably a C₁₋₇alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇alkyl group, a C₃₋₂₀heterocyclyl group, or a C₅₋₂₀aryl group, preferably a C₁₋₇alkyl group. Examples of sulfinamino groups include, but are not limited to, —NHS(═O)CH₃ and —N(CH₃)S(═O)C₆H₅.

In many cases, substituents may themselves be substituted. For example, a C₁₋₇alkyl group may be substituted with, for example, hydroxy (also referred to as a C₁₋₇hydroxyalkyl group), C₁₋₇alkoxy (also referred to as a C₁₋₇alkoxyalkyl group), amino (also referred to as a C₁₋₇aminoalkyl group), halo (also referred to as a C₁₋₇haloalkyl group), carboxy (also referred to as a C₁₋₇carboxyalkyl group), and C₅₋₂₀aryl (also referred to as a C₅₋₂₀aryl-C₁₋₇alkyl group).

Similarly, a C₅₋₂₀aryl group may be substituted with, for example, hydroxy (also referred to as a C₅₋₂₀hydroxyaryl group), halo (also referred to as a C₅₋₂₀haloaryl group), amino (also referred to as a C₅₋₂₀aminoaryl group, e.g., as in aniline), C₁₋₇alkyl (also referred to as a C₁₋₇alkyl-C₅₋₂₀aryl group, e.g., as in toluene), and C₁₋₇alkoxy (also referred to as a C₁₋₇alkoxy-C₅₋₂₀aryl group, e.g., as in anisole).

These and other specific examples of such substituted-substituents are described below.

Hydroxy-C₁₋₇alkyl: The term “hydroxy-C₁₋₇alkyl,” as used herein, pertains to a C₁₋₇alkyl group in which at least one hydrogen atom (e.g., 1, 2, 3) has been replaced with a hydroxy group. Examples of such groups include, but are not limited to, —CH₂OH, —CH₂CH₂OH, and —CH(OH)CH₂OH.

Halo-C₁₋₇alkyl group: The term “halo-C₁₋₇alkyl,” as used herein, pertains to a C₁₋₇alkyl group in which at least one hydrogen atom (e.g., 1, 2, 3) has been replaced with a halogen atom (e.g., F, Cl, Br, I). If more than one hydrogen atom has been replaced with a halogen atom, the halogen atoms may independently be the same or different. Every hydrogen atom may be replaced with a halogen atom, in which case the group may conveniently be referred to as a C₁₋₇perhaloalkyl group.” Examples of such groups include, but are not limited to, —CF₃, —CHF₂, —CH₂F, —CCl₃, —CBr₃, —CH₂CH₂F, —CH₂CHF₂, and —CH₂CF₃.

Amino-C₁₋₇alkyl: The term “amino-C₁₋₇alkyl,” as used herein, pertains to a C₁₋₇alkyl group in which at least one hydrogen atom (e.g., 1, 2, 3) has been replaced with an amino group. Examples of such groups include, but are not limited to, —CH₂NH₂, —CH₂CH₂NH₂, and —CH₂CH₂N(CH₃)₂.

Carboxy-C₁₋₇alkyl: The term “carboxy-C₁₋₇alkyl,” as used herein, pertains to a C₁₋₇alkyl group in which at least one hydrogen atom (e.g., 1, 2, 3) has been replaced with a carboxy group. Examples of such groups include, but are not limited to, —CH₂COOH and —CH₂CH₂COOH.

C₁₋₇alkoxy-C₁₋₇alkyl: The term “C₁₋₇alkoxy-C₁₋₇alkyl,” as used herein, pertains to a C₁₋₇alkyl group in which at least one hydrogen atom (e.g., 1, 2, 3) has been replaced with a C₁₋₇alkoxy group. Examples of such groups include, but are not limited to, —CH₂OCH₃, —CH₂CH₂OCH₃, and, —CH₂CH₂OCH₂CH₃.

C₅₋₂₀aryl-C₁₋₇alkyl: The term “C₅₋₂₀aryl-C₁₋₇alkyl,” as used herein, pertains to a C₁₋₇alkyl group in which at least one hydrogen atom (e.g., 1, 2, 3) has been replaced with a C₅₋₂₀aryl group. Examples of such groups include, but are not limited to, benzyl (phenylmethyl, PhCH₂—), benzhydryl (Ph₂CH—), trityl (triphenylmethyl, Ph₃C—), phenethyl (phenylethyl, Ph-CH₂CH₂—), styryl (Ph-CH═CH—), cinnamyl (Ph-CH═CH—CH₂—).

Hydroxy-C₅₋₂₀aryl: The term “hydroxy-C₅₋₂₀aryl,” as used herein, pertains to a C₅₋₂₀aryl group in which at least one hydrogen atom (e.g., 1, 2, 3) has been substituted with an hydroxy group. Examples of such groups include, but are not limited to, those derived from: phenol, naphthol, pyrocatechol, resorcinol, hydroquinone, pyrogallol, phloroglucinol.

Halo-C₅₋₂₀aryl: The term “halo-C₅₋₂₀aryl,” as used herein, pertains to a C₅₋₂₀aryl group in which at least one hydrogen atom (e.g., 1, 2, 3) has been substituted with a halo (e.g., F, Cl, Br, I) group. Examples of such groups include, but are not limited to, halophenyl (e.g., fluorophenyl, chlorophenyl, bromophenyl, or iodophenyl, whether ortho-, meta-, or para-substituted), dihalophenyl, trihalophenyl, tetrahalophenyl, and pentahalophenyl.

C₁₋₇alkyl-C₅₋₂₀aryl: The term “C₁₋₇alkyl-C₅₋₂₀aryl,” as used herein, pertains to a C₅₋₂₀aryl group in which at least one hydrogen atom (e.g., 1, 2, 3) has been substituted with a C₁₋₇alkyl group. Examples of such groups include, but are not limited to, tolyl (from toluene), xylyl (from xylene), mesityl (from mesitylene), and cumenyl (or cumyl, from cumene), and duryl (from durene).

Hydroxy-C₁₋₇alkoxy: —OR, wherein R is a hydroxy-C₁₋₇alkyl group. Examples of hydroxy-C₁₋₇alkoxy groups include, but are not limited to, —OCH₂OH, —OCH₂CH₂OH, and —OCH₂CH₂CH₂OH.

Halo-C₁₋₇alkoxy: —OR, wherein R is a halo-C₁₋₇alkyl group. Examples of halo-C₁₋₇alkoxy groups include, but are not limited to, —OCF₃, —OCHF₂, —OCH₂F, —OCCl₃, —OCBr₃, —OCH₂CH₂F, —OCH₂CHF₂, and —OCH₂CF₃.

Carboxy-C₁₋₇alkoxy: —OR, wherein R is a carboxy-C₁₋₇alkyl group. Examples of carboxy-C₁₋₇alkoxy groups include, but are not limited to, —OCH₂COOH, —OCH₂CH₂COOH, and —OCH₂CH₂CH₂COOH.

C₁₋₇alkoxy-C₁₋₇alkoxy: —OR, wherein R is a C₁₋₇alkoxy-C₁₋₇alkyl group. Examples of C₁₋₇alkoxy-C₁₋₇alkoxy groups include, but are not limited to, —OCH₂OCH₃, —OCH₂CH₂OCH₃, and —OCH₂CH₂OCH₂CH₃.

C₅₋₂₀aryl-C₁₋₇alkoxy: —OR, wherein R is a C₅₋₂₀aryl-C₁₋₇alkyl group. Examples of such groups include, but are not limited to, benzyloxy, benzhydryloxy, trityloxy, phenethoxy, styryloxy, and cimmamyloxy.

C₁₋₇alkyl-C₅₋₂₀aryloxy: —OR, wherein R is a C₁₋₇alkyl-C₅₋₂₀aryl group. Examples of such groups include, but are not limited to, tolyloxy, xylyloxy, mesityloxy, cumenyloxy, and duryloxy.

Amino-C₁₋₁₇alkyl-amino: The term “amino-C₁₋₇alkyl-amino,” as used herein, pertains to an amino group, —NR¹R², in which one of the substituents, R¹ or R², is itself a amino-C₁₋₇alkyl group (—C₁₋₇alkyl-NR³R⁴). The amino-C₁₋₇alkylamino group may be represented, for example, by the formula —NR¹—C₁₋₇alkyl-NR³R⁴. Examples of such groups include, but are not limited to, groups of the formula —NR¹(CH₂)ₙNR¹R², where n is 1 to 6 (for example, —NHCH₂NH₂, —NH(CH₂)₂NH₂, —NH(CH₂)₃NH₂, —NH(CH₂)₄NH₂, —NH(CH₂)₅NH₂, —NH(CH₂)₆NH₂), —NHCH₂NH(Me), —NH(CH₂)₂NH(Me), —NH(CH₂)₃NH(Me), —NH(CH₂)₄NH(Me), —NH(CH₂)₅NH(Me), —NH(CH₂)₆NH(Me), —NHCH₂NH(Et), —NH(CH₂)₂NH(Et), —NH(CH₂)₃NH(Et), —NH(CH₂)₄NH(Et), —NH(CH₂)₅NH(Et), and —NH(CH₂)₆NH(Et).

Bidentate Substituents

The term “bidentate substituents,” as used herein, pertains to substituents which have two points of covalent attachment, and which act as a linking group between two other moieties.

In some cases (A), a bidentate substituent is covalently bound to a single atom. In some cases (B), a bidentate substituent is covalently bound to two different atoms, and so serves as a linking group therebetween.

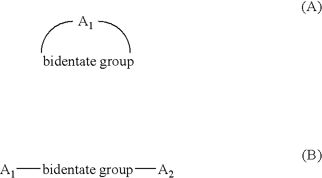

Within (B), in some cases (C), a bidentate substituent is covalently bound to two different atoms, which themselves are not otherwise covalently linked (directly, or via intermediate groups). In some cases (D), a bidentate substituent is covalently bound to two different atoms, which themselves are already covalently linked (directly, or via intermediate groups); in such cases, a cyclic structure results. In some cases, the bidentate group is covalently bound to vicinal atoms, that is, adjacent atoms, in the parent group.

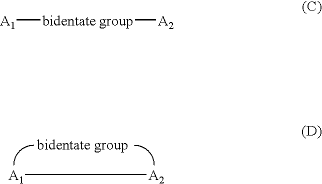

In some cases (A and D), the bidentate group, together with the atom(s) to which it is attached (and any intervening atoms, if present) form an additional cyclic structure. In this way, the bidentate substituent may give rise to a cyclic or polycyclic (e.g., fused, bridged, spiro) structure, which may be aromatic.

Examples of bidentate groups include, but are not limited to, C₁₋₇alkylene groups, C₃₋₂₀heterocyclylene groups, and C₅₋₂₀arylene groups, and substituted forms thereof.

Includes Other Forms

Unless otherwise specified, included in the above are the well known ionic, salt, solvate, and protected forms of these substituents. For example, a reference to carboxylic acid (—COOH) also includes the anionic (carboxylate) form (—COO—), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Isomers, Salts. Solvates, Protected Forms, and Prodrugs

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

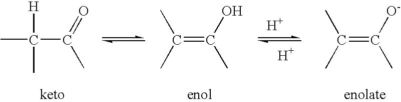

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³.

Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyidimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O•).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl (e.g., -Me, -Et, -nPr, -iPr, -nBu, -sBu, -iBu, -tBu);

C₁₋₇aminoalkyl (e.g., aminoethyl; 2-(N,N-diethylamino)ethyl; 2-(4-morpholino)ethyl); and acyloxy-C₁₋₇alkyl

(e.g., acyloxymethyl;

acyloxyethyl;

pivaloyloxymethyl;

acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl;

1-cyclohexyloxy-carbonyloxyethyl;

(4-tetrahydropyranyloxy)carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

Uses

The present invention pertains to cannabinoid receptor inverse agonists and cannabinoid receptor neutral antagonists, as described herein, which inhibit osteoclasts, for example, inhibit of the survival, formation, and/or activity of osteoclasts, and/or which inhibit bone resorption. Therefore, the compounds may also be referred to as “osteoclast inhibitors” and/or “bone resorption inhibitors.”

One of ordinary skill in the art is readily able to determine whether or not a candidate compound inhibits the survival, formation, and/or activity of osteoclasts and/or inhibits bone resorption. For example, suitable methods which may conveniently be used in order to assess the inhibitory effects offered by a particular compound are described in the examples below.

Use in Methods of Inhibition

One aspect of the invention pertains to a method of inhibiting osteoclast survival, formation, and activity, in vitro or in vivo, comprising contacting an osteoclast with an effective amount of a cannabinoid receptor inverse agonist or a cannabinoid receptor neutral antagonist, as described herein.

One aspect of the invention pertains to a method of inhibiting bone resorption, in vitro or in vivo, comprising contacting cells in the bone microenvironment with a therapeutically-effective amount of a cannabinoid receptor inverse agonist or a cannabinoid receptor neutral antagonist, as described herein.

The term “cells in the bone microenvironment,” as used herein, pertains to cells such as osteoblasts, osteoclasts, osteocytes and bone marrow stromal cells, which are located in close proximity to bone (e.g., within one hundred micrometers of the bone surface).

Bone Disorders

The cannabinoid receptor inverse agonists and cannabinoid receptor neutral antagonists, as described herein, are useful in the treatment of bone disorders, for example, conditions mediated by osteoclasts (e.g., by increased osteoclast activity) (as “osteoclast inhibitors”), and/or conditions characterised by increased bone resorption (as “bone resorption inhibitors”).

In one embodiment, the bone disorder is characterised by increased osteoclast activity.

In one embodiment, the bone disorder is characterised by increased bone resorption.

In one embodiment, the bone disorder is associated with a genetic predisposition, sex hormone deficiency, or ageing. For example, in one embodiment, the bone disorder is characterised by increased bone resorption, and is associated with a genetic predisposition, sex hormone deficiency, or ageing.

In one embodiment, the bone disorder is not associated with inflammation. For example, in one embodiment, the bone disorder is characterised by increased bone resorption, and is not associated with inflammation. For example, in one embodiment, the bone disorder is characterised by increased bone resorption; and is associated with a genetic predisposition, sex hormone deficiency, or ageing; and is not associated with inflammation.

For example, in one embodiment, the bone disorder is not associated with rheumatoid arthritis, ankylosing spondylitis, or inflammatory bowel disease. For example, in one embodiment, the bone disorder is characterised by increased bone resorption, and is not associated with rheumatoid arthritis, ankylosing spondylitis, or inflammatory bowel disease. For example, in one embodiment, the bone disorder is characterised by increased bone resorption; and is associated with a genetic predisposition, sex hormone deficiency, or ageing; and is not associated with rheumatoid arthritis, ankylosing spondylitis, or inflammatory bowel disease.

Examples of such bone disorders include, but are not limited to, the following:

diseases of the skeleton, including but not limited to, pathologically low bone mineral density, such as:

osteoporosis (including, e.g., steroid induced osteoporosis) (e.g., osteoporosis not associated with inflammation);

osteoarthritis;

Paget's disease of bone (osteitis deformans);

hypercalcaemia caused by conditions associated with increased bone resorption, including, but not limited to: vitamin D intoxication, primary or tertiary hyperparathyroidism, immobilisation, and sarcoidosis;

neoplasia of bones, both as a primary tumour and as metastases, including but not limited to, osteosarcoma and osteoma (Zheng et al., 1998, J. Cell Biochem., Vol. 70, p. 121) and cancer associated bone disease (e.g., hypercalcaemia of malignancy, bone metastases, osteolytic bone metastases, multiple myeloma, breast carcinoma).

In one embodiment, the bone disorder is osteoporosis (e.g., osteoporosis not associated with inflammation; e.g., osteoporosis associated with a genetic predisposition, sex hormone deficiency, or ageing), cancer associated bone disease, and Paget's disease of bone.

In one embodiment, the bone disorder is osteoporosis (e.g., osteoporosis not associated with inflammation and/or osteoporosis associated with a genetic predisposition, sex hormone deficiency, or ageing).

Use in Methods of Treatment

One aspect of the present invention pertains to a method of treating a bone disorder, as described herein, comprising administering to a patient in need of treatment thereof a therapeutically effective amount of a cannabinoid receptor inverse agonist or a cannabinoid receptor neutral antagonist, as described herein, preferably in the form of a pharmaceutical composition.

One aspect of the present invention pertains to a method for the treatment of a condition mediated by osteoclasts (e.g., increased osteoclast activity) and/or characterised by (e.g., increased) bone resorption, as described herein, comprising administering to a subject suffering from said condition a therapeutically-effective amount of a cannabinoid receptor inverse agonist or a cannabinoid receptor neutral antagonist, as described herein, preferably in the form of a pharmaceutical composition.

One aspect of the present invention pertains to a method for the treatment of a condition mediated by osteoclasts (e.g., increased osteoclast activity), as described herein; comprising administering to a subject suffering from said condition a therapeutically-effective amount of a cannabinoid receptor inverse agonist or a cannabinoid receptor neutral antagonist, as described herein, preferably in the form of a pharmaceutical composition.

One aspect of the present invention pertains to a method for the treatment of a condition characterised by (e.g., increased) bone resorption, as described herein, comprising administering to a subject suffering from said condition a therapeutically-effective amount of a cannabinoid receptor inverse agonist or a cannabinoid receptor neutral antagonist, as described herein, preferably in the form of a pharmaceutical composition.

One aspect of the present invention pertains to a method for the treatment of osteoporosis (e.g., osteoporosis not associated with inflammation; e.g., osteoporosis associated with a genetic predisposition, sex hormone deficiency, or ageing), cancer associated bone disease, or Paget's disease of bone, comprising administering to a subject suffering from said condition a therapeutically-effective amount of a cannabinoid receptor inverse agonist or a cannabinoid receptor neutral antagonist, as described herein, preferably in the form of a pharmaceutical composition.

Use in Methods of Therapy

One aspect of the present invention pertains to a cannabinoid receptor inverse agonist or a cannabinoid receptor neutral antagonist, as described herein, for use in a method of treatment of the human or animal body by therapy.

One aspect of the present invention pertains to a cannabinoid receptor inverse agonist or a cannabinoid receptor neutral antagonist, as described herein, for use in a method of treatment of a condition mediated by osteoclasts (e.g., increased osteoclast activity) and/or characterised by (e.g., increased) bone resorption, as described herein, of the human or animal body by therapy.

One aspect of the present invention pertains to a cannabinoid receptor inverse agonist or a cannabinoid receptor neutral antagonist, as described herein, for use in a method of treatment of a condition mediated by osteoclasts (e.g., increased osteoclast activity), as described herein, of the human or animal body by therapy.

One aspect of the present invention pertains to a cannabinoid receptor inverse agonist or a cannabinoid receptor neutral antagonist, as described herein, for use in a method of treatment of a condition characterised by (e.g., increased) bone resorption, as described herein, of the human or animal body by therapy.

One aspect of the present invention pertains to a cannabinoid receptor inverse agonist or a cannabinoid receptor neutral antagonist, as described herein, for use in a method of treatment of osteoporosis (e.g., osteoporosis not associated with inflammation; e.g., osteoporosis associated with a genetic predisposition, sex hormone deficiency, or ageing), cancer associated bone disease, and Paget's disease of bone, of the human or animal body by therapy.

Use in the Manufacture of Medicaments

One aspect of the present invention pertains to use of a cannabinoid receptor inverse agonist or a cannabinoid receptor neutral antagonist, as described herein, for the manufacture of a medicament for use in the treatment of a condition mediated by osteoclasts (e.g., increased osteoclast activity) and/or characterised by (e.g., increased) bone resorption, as described herein.

One aspect of the present invention pertains to use of a cannabinoid receptor inverse agonist or a cannabinoid receptor neutral antagonist, as described herein, for the manufacture of a medicament for use in the treatment of a condition mediated by osteoclasts (e.g., increased osteoclast activity), as described herein.

One aspect of the present invention pertains to use of a cannabinoid receptor inverse agonist or a cannabinoid receptor neutral antagonist, as described herein, for the manufacture of a medicament for use in the treatment of a condition characterised by (e.g., increased) bone resorption, as described herein.

One aspect of the present invention pertains to use of a cannabinoid receptor inverse agonist or a cannabinoid receptor neutral antagonist, as described herein, for the manufacture of a medicament for use in the treatment of osteoporosis (e.g., osteoporosis not associated with inflammation; e.g., osteoporosis associated with a genetic predisposition, sex hormone deficiency, or ageing), cancer associated bone disease, and Paget's disease of bone.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy.

Other Uses

Cannabinoid receptor inverse agonists and cannabinoid receptor neutral antagonists, as described herein, may also be used as cell culture additives to inhibit osteoclasts, for example, to inhibit the survival, formation, and/or activity of osteoclasts.

Cannabinoid receptor inverse agonists and cannabinoid receptor neutral antagonists, as described herein, may also be used as part of an in vitro assay, for example, in order to determine whether a candidate host is likely to benefit from treatment with the compound in question.

Cannabinoid receptor inverse agonists and cannabinoid receptor neutral antagonists, as described herein, may also be used as a standard, for example, in an assay, in order to identify other active compounds, other osteoclast inhibitors, other bone resorption inhibitors, etc.

Methods of Identifying Bone Disorder Therapeutic Agents

One aspect of the present invention pertains to a method of identifying a bone disorder therapeutic agent on the basis that it has one or more of the functional characteristics described herein (e.g., is a cannabinoid receptor inverse agonist or a cannabinoid receptor neutral antagonist; is a CB1 or CB2 inverse agonist; etc.).

For example, one aspect of the present invention pertains to a method of identifying a bone disorder therapeutic agent on the basis that it is a cannabinoid receptor inverse agonist or a cannabinoid receptor neutral antagonist and has a cannabinoid receptor inhibition constant (Ki) of 10 μM or less, as described herein.

In one embodiment, the method further comprises the step of testing, demonstrating, and/or determining the activity and/or efficacy of the bone disorder therapeutic agent, using suitable means, for example, J774 murine macrophage viability assays, the rabbit osteoclast cultures, osteoblast bone marrow co-culture assays, etc.

One aspect of the present invention pertains to a bone disorder therapeutic agent identified by such methods.

Routes of Administration

The active compound or pharmaceutical composition comprising the active compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The Subject

The subject may be an animal, a chordate, a vertebrate, a mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject may be any of its forms of development, for example, a foetus.

In one preferred embodiment, the subject is a human.

Formulations

While it is possible for the active compound (e.g., cannabinoid receptor inverse agonist or cannabinoid receptor neutral antagonist, as described herein) to be administered alone, it is preferable to present it as a pharmaceutical formulation (e.g., composition, preparation, medicament) comprising at least one active compound, as defined above, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one active compound, as defined above, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more active compounds and optionally one or more other pharmaceutically acceptable ingredients, including, for example, penetration, permeation, and absorption enhancers. Formulations may also suitably be provided in the form of a depot or reservoir.

The active compound may be dissolved in, suspended in, or admixed with one or more other pharmaceutically acceptable ingredients. The active compound may be presented in a liposome or other microparticulate which is designed to target the active compound, for example, to blood components or one or more organs.

Formulations suitable for oral administration (e.g, by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the active compound in a flavored basis, usually sucrose and acacia or tragacanth. Pastilles typically comprise the active compound in an inert matrix, such as gelatin and glycerin, or sucrose and acacia. Mouthwashes typically comprise the active compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the active compound and a paraffinic or a water-miscible ointment base.

Creams are typically prepared from the active compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions are typically prepared from the active compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the active compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the active compound is dissolved or suspended in a suitable carrier, especially an aqueous solvent for the active compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the active compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active compound in the liquid is from about 1 ng/mL to about 10 μg/mL, for example from about 10 ng/ml to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Dosage

It will be appreciated by one of skill in the art that appropriate dosages of the cannabinoid receptor inverse agonists or cannabinoid receptor neutral antagonists, and compositions comprising them, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 μg to about 250 mg (more typically about 100 μg to about 25 mg) per kilogram body weight of the subject per day. Where the active compound is a salt, an ester, an amide, a prodrug, or the like, the amount administered is calculated on the basis of the parent compound and so the actual weight to be used is increased proportionately.

Kits

One aspect of the invention pertains to a kit comprising (a) an active compound (e.g., cannabinoid receptor inverse agonist or cannabinoid receptor neutral antagonist), as described herein, or a composition comprising an active compound, as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

Mechanism of Action

The inventors have shown that inverse agonists (as discussed above, inverse agonists can both block the action of the agonist and attenuate receptor constitutive activity) of the cannabinoid receptors CB1 and CB2 have potent inhibitory effects on survival of J774 macrophages (an established model system to test for compounds which inhibit osteoclastic activity; see, e.g., Rogers et al., 1996) and on the survival and resorptive activity of isolated rabbit osteoclasts. This identifies the endocannabinoid system as a novel therapeutic target for the treatment of bone diseases. The data are consistent with a model whereby the survival and activity of osteoclasts is regulated by CB receptor activation.

More particularly, the inventors have found that inverse agonists of the CB1/CB2 receptors (AM251, AM630, SR144528, and JTE-907) modulated J774 survival in a concentration dependent manner. In addition, AM251 and SR144528 were found to inhibit survival and resorptive activity of authentic rabbit osteoclasts and to completely reverse ovariectomy-induced bone loss in a mouse model. These data suggest that CB receptors are the molecular target and that inverse agonists of CB receptors have inhibitory effects on bone resorption.

The data presented herein indicate that the CB inverse agonists AM251, AM630, SR144528, and JTE-907 have potent inhibitory effects on J774 survival and rabbit osteoclast survival and resorptive activity. It is known that CB2 receptor expression predominates in cells of macrophage lineage. These data therefore suggest that inhibition of osteoclast survival by AM251, AM630, SR144528, and JTE-907 may be CB2 receptor mediated. However, inverse agonism at the CB1 receptor or synergistic effect of CB1 and CB2 receptors in osteoclasts cannot be ruled out. These compounds are known to be inverse agonists at CB1 and CB2 receptors. In terms of affinity, AM251 is CB1 selective and AM630, SR144528 and JTE-907 are CB2 selective; however, the relative potency of these compounds as inverse agonists at each subtype is not known. Thus, although AM251 is CB1 selective, it also has affinity for the CB2 receptor and may also be a potent CB2 receptor inverse agonist.

It is important to point out that tone in the endocannabinoid system may exist either via constitutively active CB receptors or via an ongoing release of endogenous CB receptor agonists. It is known that J774 macrophages synthesise and release endocannabinoids and contain the enzymes responsible for the endocannabinoid inactivation (see, e.g., Di et al., 1996). In view of this, it is possible that autocrine release of endocannabinoids plays a role in J774 survival and that inhibition of receptor binding by AM251 or related compounds will compromise J774 survival (and by implication, osteoclast survival) by disrupting this autocrine loop.

Whilst the data presented herein suggest that the CB2 receptor is the target site of interest, it is important to note that the pharmacology of anandamide and other endocannabinoids is highly complex. Thus, the pharmacological characteristics of these eicosanoids indicate that their actions may be mediated by cannabinoid CB1 and CB2 receptors and by putative non-CB1, non-CB2 or “CB2-like” receptors (see, e.g., Pertwee et al., 2002; Di et al., 1998; Di et al., 2002).

Anandamide also interacts with vanilloid TRPV1 receptors both directly (see, e.g., Zygmunt et al., 1999; Ross et al., 2001) and indirectly via metabolites (see, e.g., Craib et al., 2001). Anandamide is subject to rapid intracellular hydrolysis by fatty acid amide hydrolase (FAAH) to yield arachidonic acid and ethanolamide. Recent work suggests that anandamide may also be metabolised by a range of oxygenase enzymes already known to convert arachidonic acid to a number of potent biologically active compounds (see, e.g., Kozak et al., 2002). In addition, SR141716A, which is structurally related to AM251, interacts with both TRPVR1 receptors and non-CB1 receptors (see, e.g., Pertwee et al., 2002). Consequently, it is possible that cannabinoid-related compounds such AM251, AM630, SR144528, and JTE-907 may be acting at a novel target site of action, which may be a receptor, ion-channel or metabolic enzyme. Alternatively, the compounds may be achieving inhibition via an interaction with both CB1 and CB2 receptors: AM251 and AM630 both interact with both the CB1 and the CB2 receptor. There is evidence that of synergism between CB1 and CB2 receptor in effecting the anti-inflammatory action of endocannabinoids (see, e.g., Calignano et al., 1998).

The following Table summarises the results obtained in the J774 viability assay. The data demonstrate that:

(a) the inverse agonists AM251 and AM630, which are from different chemical classes, inhibit J774 viability;

(b) the endocannabinoid anandamide, which is an antagonist at the CB2 receptor, also inhibits J774 viability;

(c) the non-selective CB1/CB2 receptor agonists WIN55212 and CP55940 do not affect J774 viability;

(d) the CB2 selective agonist JWH-133 also does not affect viability.

The data therefore suggest that cannabinoid receptor inverse agonist/antagonists are effective in the J774 viability assay.

TABLE 2

| | | | IC₅₀ in J774 |
|---|---|---|---|
| Compound | Chemical Class | CB receptor function | cells |
| Anandamide | Endocannabinoid | CB1 agonist | 40 μM |
| | | CB2 partial agonist/ | |
| | | antagonist | |
| AM251 | Pyrazole | CB1 inverse agonist | 5 μM |
| AM630 | Indole | CB1 inverse agonist | 20 μM |
| | | CB2 inverse agonist | |
| WIN55212 | Indole | CB1 agonist | |
| | | CB2 agonist | inactive |
| JWH 133 | Classical | CB2-selective agonist | inactive |
| | Cannabinoid | | |
| CP55940 | Classical | CB1 agonist | inactive |
| | Cannabinoid | CB2 agonist | |
| SR144528 | Pyrazole | CB2 inverse agonist | 9 uM |
| JTE-907 | 2-Oxoquinoline | CB2 inverse agonist | 25 uM |

### EXAMPLES

The following examples are provided solely to illustrate the present invention and are not intended to limit the scope of the invention, as described herein.

### SYNTHESIS EXAMPLES

### Synthesis Example 1-1

### Sodium salt of 4-(4-chloro-3-methyl-phenyl)-4-oxido-2-oxo-but-3-enoic acid ethyl ester

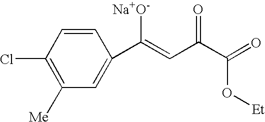

Sodium (1.36 g) was dissolved in methanol (35 ml) and cooled to room temperature. 4-Chloro-3-methylacetophenone (10 g) was added followed by diethyl oxalate (8 ml) in methanol (10 ml). A slurry resulted. Addition of methanol (10 ml) failed to fluidise the mixture and it was left to stand for 2 hrs. Diethyl ether (150 ml) was added and the suspension stirred for 1 hr. Filtration and washing with ether gave the title product as a pale green solid.

### Synthesis Example 1-2

### 5-(4-Chloro-3-methyl-phenyl)-1H-pyrazole-3-carboxylic acid methyl ester

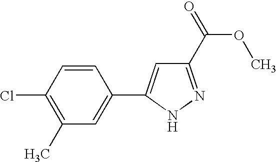

The 4-(4-chloro-3-methyl-phenyl)-4-oxido-2-oxo-but-3-enoic acid ethyl ester (8.8 g) was dissolved in acetic acid (100 ml) with gentle heating. After cooling to room temperature, hydrazine monohydrate (1.7 ml) was added dropwise. The solution was refluxed for 4.5 hours and then stirred overnight, after which a solid had been precipitated. The solid was filtered and washed with water. Recrystallisation from ethanol gave the title product as a white solid.

### Synthesis Example 1-3

### 5-(4-Chloro-3-methyl-phenyl)-1-(4-methyl-benzyl)-1H-pyrazole-3-carboxylic acid methyl ester

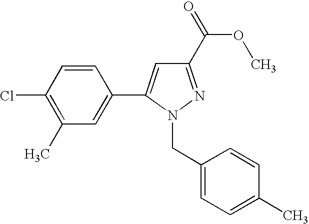

5-(4-Chloro-3-methyl-phenyl)-1H-pyrazole-3-carboxylic acid methyl ester (2.5 g) was suspended in toluene (25 ml) and added-dropwise to a suspension of 60% NaH (0.95 g) in toluene (25 ml). The mixture was stirred at 65° C. for 1 hour. 4-Methylbenzyl bromide (2 g) was added and the suspension refluxed overnight. After cooling, the reaction was quenched with saturated NH₄Cl (50 ml). The organic phase was separated and washed with saturated NaCl, dried over MgSO₄ and evaporated to give a clear oil which solidified on standing. Purified by column chromatography (toluene: DCM: ethyl acetate, 8:1:1). δ_{C} (CDCl₃, 62.9 MHz): 20.0, 21.1, 52.1, 54.1, 109.5, 126.9, 127.6, 128.0, 129.4, 131.6, 133.5, 135.5, 136.7, 137.6, 142.8, 144.4 and 162.9. δ_{H} (CDCl₃, 250 MHz): 2.30 (3H, s), 2.33 (3H, s), 3.94 (3H, s), 5.33 (2H, s) and 6.84-7.35 (8H, m).

### Synthesis Example 1-4

### 5-(4-Chloro-3-methyl-phenyl)-1-(4-methyl-benzyl)-1H-pyrazole-3-carboxylic acid

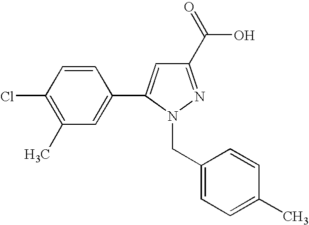

5-(4-Chloro-3-methyl-phenyl)-1-(4-methyl-benzyl)-1H-pyrazole-3-carboxylic acid methyl ester (1.2 g) was dissolved in ethanol (50 ml). A solution of KOH (0.35 g) in water (10 ml) was added and the solution refluxed overnight. The solvent was evaporated and the white residue dissolved in water (100 ml), washed with ether, acidified to pH 2 with conc. HCl. Filtration gave the title product as a white solid (0.8 g). δ_{C} (CDCl₃, 62.9 MHz): 20.1, 21.1, 54.2, 109.9, 127.0, 127.7, 127.9, 128.9, 129.4, 131.6, 133.3, 135.6, 136.7, 137.8, 142.3, 144.8 and 166.4.

### Synthesis Example 1-5

### 5-(4-Chloro-3-methyl-phenyl)-1-(4-methyl-benzyl)-1H-pyrazole-3-carbonyl chloride

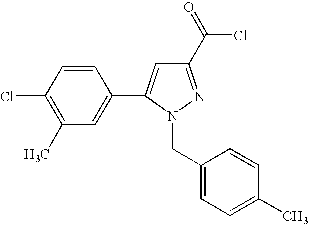

5-(4-Chloro-3-methyl-phenyl)-1-(4-methyl-benzyl)-1H-pyrazole-3-carboxylic acid (0.8 g) was placed in toluene (30 ml). Thionyl chloride (2 ml) was added and the mixture refluxed for 4 hours. The solvent was evaporated to give a yellow oil. The oil was dissolved in 30 ml toluene and again refluxed with thionyl chloride for 2 hours. Evaporation of the solvent gave the title product.

### Synthesis Example 1-6

### (1S)-1,3,3-Trimethyl-bicyclo[2.2.1]heptan-2-one oxime

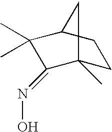

Hydroxylamine hydrochloride (11.5 g) and sodium acetate (20.5 g) were dissolved in water (100 ml), added to a solution of fenchone (19 g) in methanol (60 ml) and refluxed for 48 hours. After cooling, the large white needles were filtered, washed with water and dried under vacuum to give the title product (20 g).

### Synthesis Example 1-7

### (1S)-1,3,3-trimethylbicyclo[2.2.1]hept-2-endo,exo-yl-amine

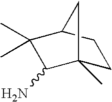

(1S)-1,3,3-Trimethyl-bicyclo[2.2.1]heptan-2-one oxime (5 g) was dissolved in acetic acid (35 ml) and chilled in an ice bath. Activated Raney Nicitel (15 g) was added, the mixture warmed to room temperature and stirred overnight under an atmosphere of hydrogen. The catalyst was filtered off using celite and washed through with a further portion of acetic acid (20 ml). Ice water (200 ml) was added to the filtrate and the pH adjusted to 7 using conc. NaOH solution. A green emulsion was produced. The emulsion was extracted with ether. The ether layer was dried and evaporated to give a white sludge. Addition of ether gave a white solid. Filtration gave the title product as a 1:1 mixture of the two isomers (1.2 g). δ_{C} (CDCl₃, 62.9 MHz): 17.4, 19.4, 20.5, 23.2, 23.7, 25.3, 25.8, 26.0, 26.5, 30.9, 35.8, 38.7, 41.2, 42.5, 42.7, 48.0, 48.4, 48.9, 66.1 and 67.0.

### Synthesis Example 1-8

### 5-(4-Chloro-3-methyl-phenyl)-1-(4-methyl-benzyl)-1H-pyrazole-3-carboxylic acid ((1S)-1,3,3-trimethylbicyclo[2.2.1]hept-2endo,exo-yl)-amide

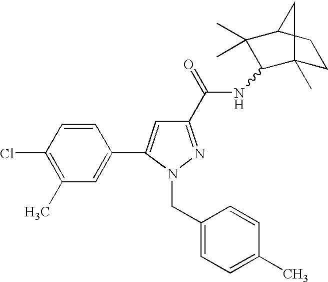

(1S)-1,3,3-trimethylbicyclo[2.2.1]hept-2-endo,exo-yl-amine (0.6 g) was suspended in DCM (20 ml) and 2 ml triethylamine. 5-(4-Chloro-3-methyl-phenyl)-1-(4-methyl-benzyl)-1H-pyrazole-3-carbonyl chloride (0.8 g) was dissolved in DCM (30 ml), added dropwise to the amine suspension and left to stir overnight at room temperature. The solvent was evaporated and the residue extracted with ethyl acetate. The organic phase was filtered and washed with saturated NaHCO₃ and NaCl solutions, dried over Na₂SO₄, and evaporated to give an oil. The product was purified by column chromatography using petrol:ethyl acetate (9:1). Four spots were seen by thin layer chromatography (TLC), the central pair being the desired product endo/exo isomers. Repeated column chromatography gave the exo-isomer (first fraction) (220 mg) and the endo-isomer (180 mg) as amorphous solids.

Exo isomer: δ_{C} (CDCl₃, 62.9 MHz): 19.8, 20.1, 21.1, 21.3, 26.1, 27.4, 31.0, 39.5, 42.7, 48.2, 48.7, 53.6, 63.0, 107.1, 126.7, 127.5, 128.5, 129.4, 131.5, 133.9, 135.3, 136.6, 137.3, 144.7, 146.3 and 162.6. δ_{H} (CDCl₃, 250 MHz): 0.87 (3H, s), 1.11 (3H, s), 1.17 (3H, s), 1.23-1.77 (7H, m), 2.32 (6H, s), 3.80 (1H, d, J 9.8), 5.27 (2H, s), 5.29 (1H, m), 6.82 (1H, s), 6.93 (2H, d, J 7.94), 7.02 (2H, d, J 7.94), 7.09 (2H, m) and 7.33 (1H, d, J 8.24).

Endo isomer: δ_{C} (CDCl₃, 62.9 MHz): 17.9, 20.1, 21.1, 24.6, 25.6, 26.9, 35.5, 42.3, 43.5, 48.3, 48.4, 53.6, 64.4, 107.1, 126.7, 127.5, 128.5, 129.4, 131.5, 133.9, 135.3, 136.6, 137.3, 144.7, 146.3 and 162.6. δ_{H} (CDCl₃, 250 MHz): 0.95 (3H, s), 1.08 (3H, s), 1.15 (3H, s), 1.20-1.71 (7H, m), 2.32 (6H, s), 3.60 (1H, d, J 10.4), 5.27 (2H, s), 6.83 (1H, s), 6.93 (2H, d, J 7.94), 7.02 (2H, d, J 7.94), 7.09 (2H, m) 7.24 (1H, m) and 7.33 (1H, d, J 8.24).

### Synthesis Example 2

### 5-(4=Chloro-3-methyl-phenyl)-1-(4-methyl-benzyl)-1H-pyrazole-3-carboxylic acid piperidin-1-yl amide

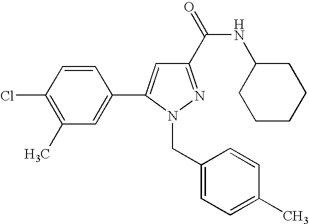

N-Aminopiperidine (0.5 g) was suspended in DCM (20 ml) and 1 ml triethylamine. 5-(4-Chloro-3-methyl-phenyl)-1-(4-methyl-benzyl)-1H-pyrazole-3-carbonyl chloride (0.4 g) (see above) was dissolved in DCM (5 ml), added dropwise to the suspension, and left to stir overnight at room temperature. The solvent was evaporated and the residue extracted with ethyl acetate. The organic phase was filtered and washed with saturated NaHCO₃ and NaCl solutions, dried over Na₂SO₄, and evaporated to give the title compound as an oil. The product was purified by column chromatography using petrol: ethyl acetate (1:1) to give a white solid (65%). δ_{C} (CDCl₃, 62.9 MHz): 20.1, 21.1, 23.3, 25.3, 53.5, 57.2, 107.7, 126.6, 127.4, 128.3, 129.5, 131.2, 131.5, 133.7, 135.4, 136.7, 137.6, 144.7, 145.6 and 159.2. δ_{H} (CDCl₃, 250 MHz): 1.43 (2H, m), 1.75 (4H, m), 2.32 (6H, s), 2.88 (4H, m), 5.25 (2H, s), 6.88 (1H, s), 6.91 (2H, d, J 8.24), 7.02 (2H, d, J 8.24), 7.09 (2H, d, J 7.02), 7.25 (1H, m), 7.33 (1H, d, J 8.24) and 7.66 (1H, br s).

### Synthesis Example 3

### 5-(4-Chloro-3-methyl-phenyl)-1-(4-methyl-benzyl)-1H-pyrazole-3-carboxylic acid (benzo[1,3]dioxol-5-yl-methyl) amide

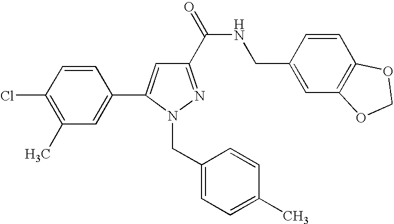

3,4-Methylenedioxybenzylamine (0.5 g) was suspended in DCM (20 ml) and 1 ml triethylamine. 5-(4-Chloro-3-methyl-phenyl)-1-(4-methyl-benzyl)-1H-pyrazole-3-carbonyl chloride (0.4 g) (see above) was dissolved in DCM (5 ml), added dropwise to the suspension and left to stir overnight at room temperature. The solvent was evaporated and the residue extracted with ethyl acetate. The organic phase was filtered and washed with saturated NaHCO₃ and NaCl solutions, dried over Na₂SO₄ and evaporated to give the title compound as an oil. The product was purified by column chromatography using petrol:ethyl acetate (9:1). δ_{C} (CDCl₃, 62.9 MHz): 20.1, 21.1, 43.0, 53.5, 101.0, 107.3, 108.3, 108.6, 121.2, 126.6, 127.5, 128.3, 129.5, 131.2, 131.5, 132.4, 133.7, 135.4, 136.7, 137.7, 144.9, 146.0, 146.9, 147.9 and 161.8. δ_{H} (CDCl₃, 250 MHz): 2.31 (3H, s), 2.34 (3H, s), 4.52 (2H, d, J 5.8), 5.23 (2H, s), 5.92 (2H, s), 6.76-7.12 (9H, m) 7.21 (1H, m), and 7.34 (1H, d, J 8.24).

### Synthesis Example 4-1

### 3-Hydroxy-4-methoxy-2-nitro-benzaldehyde

### 3-Hydroxy-4-methoxy-6-nitro-benzaldehyde

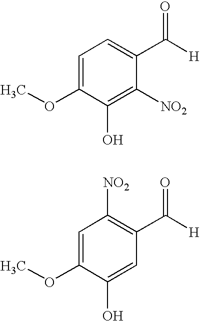

3-Hydroxy-4-methoxy-benzaldehyde (15.2 g) was dissolved in THF (150 ml). NaNO₃ (8.5 g) and lanthanum nitrate hexahydrate (8.7 g) were added. A mixture of conc. HCl (35 ml) and water (35 ml) was added dropwise over 20 minutes and the reaction mixture stirred at room temperature for 1 hour. The mixture was extracted with ethyl acetate and diethyl ether. Each of the organic phases was washed with water (50 ml) saturated Na₂CO₃ (50 ml) and saturated NaCl (50 ml). Drying and evaporation gave the title compounds as a crude mixture.

### Synthesis Example 4-2

### 4-Methoxy-2-nitro-3-pentyloxy-benzyaldehyde 4-Methoxy-6-nitro-3-pentyloxy-benzyaldehyde

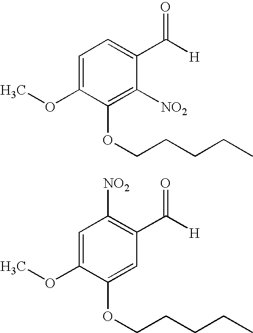

3-Hydroxy-4-methoxy-2-nitro-benzaldehyde (as a crude mixture of products from the previous example) (12.3 g) was dissolved in DMF (20 ml). Bromopentane (11.3 g) and anhydrous K₂CO₃ (12.9 g) were added. The suspension was stirred in a boiling water bath for 5 hours. The mixture was cooled, filtered and saturated NH₄Cl (200 ml) added to the filtrate. The filtrate was extracted with ethyl acetate and this was washed with saturated NaCl and dried. Evaporation and purification with column chromatography (petrol:ethyl acetate, 5:1) gave the title compounds as a crude mixture.

A crude sample of 4-Methoxy-2-nitro-3-pentyloxy-benzyaldehyde was obtained, contaminate with starting materials.

A pure sample of 4-Methoxy-6-nitro-3-pentyloxy-benzyaldehyde was obtained as yellow crystals by column chromatography (petrol:ethyl acetate, 6:1) or by repeated crystallisation with petrol/diethyl ether. δ_{C} (CDCl₃, 62.9 MHz): 13.9, 22.4, 27.9, 28.5, 56.8, 69.9, 107.3, 110.5, 125.6, 143.6, 152.5, 153.0, and 187.8.

### Synthesis Example 4-3

### 2-(4-Methoxy-2-nitro-3-pentyloxy-benzylidene) malonic acid

### 2-(4-Methoxy-6-nitro-3-pentyloxy-benzylidene) malonic acid

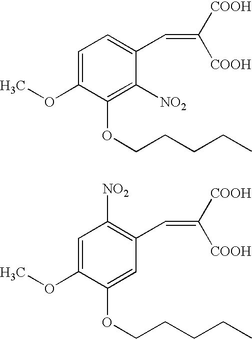

The mixture of products from the previous example (6 g) were dissolved in acetic acid (65 ml). Malonic acid (7 g) was added and the mixture stirred at 60° C. for 24 hours. The solvent was evaporated with the addition of toluene (2×100 ml). Water was added to the residue followed by NaOH solution to adjust the pH to 8, which allowed the product to go into solution. The aqueous was washed with ethyl acetate and then re-acidified with conc. HCl. The aqueous was extracted with ethyl acetate and the organic phase washed with saturated NaCl, dried and evaporated to give a mixture of the title compounds as a red solid.

### Synthesis Example 4-4

### 2-(4-Methoxy-2-nitro-3-pentyloxy-benzylidene) malonic acid methyl diester

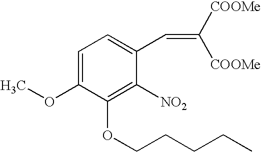

The crude mixture from the previous example (6 g) was dissolved in DMF (40 ml). Methyl iodide (4.4 ml) was added followed by K₂CO₃ (6.65 g) and the mixture was stirred at room temperature for 4 hrs. A saturated solution of NH₄Cl (200 ml) was added and the mixture extracted with ethyl acetate. The organic was washed with water, saturated NaCl and dried (Na₂SO₄). Evaporation gave a brown solid. Repeated crystallisations from ether/petrol gave 2-(4-methoxy-6-nitro-3-pentyloxy-benzylidene) malonic acid methyl diester as yellow needles δ_{C} (DMSO, 62.9 MHz): 14.0, 22.4, 27.9, 28.5, 52.6, 52.8, 56.5, 69.8, 108.0, 112.0, 124.4, 127.5, 139.7 142.2, 149.8, 153.0, 163.9 and 166.0 and the title compound as a crude red oil.

### Synthesis Example 4-5

### 7-Methoxy-2-oxo-8-pentyloxy-1,2-dihydro-quinoline-3-carboxylic acid methyl ester

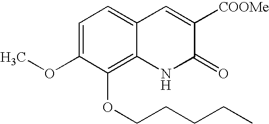

2-(4-Methoxy-2-nitro-3-pentyloxy-benzylidene) malonic acid methyl diester (5 g) was dissolved in acetic acid (60 ml) and water (4 ml) and stirred at 60° C. Reduced iron (6 g) was added and the mixture stirred at 90° C. for 2 hours. The mixture was filtered and water (200 ml) was added. The solution was extracted with ethyl acetate and DCM. Each of the organic phases was washed with 1% HCl and saturated NaCl solutions. The organic phases were combined and evaporated. The product was dissolved in DCM and purified by column chromatography. The column was flushed with ethyl acetate: petrol, 1:1, and the product then eluted using ethanol. Evaporation gave the title compound as a white solid (1.6 g). δ_{C} (CDCl₃, 62.9 MHz): 14.0, 22.5, 27.9, 29.8, 52.5, 56.3, 73.8, 108.5, 113.3, 119.6, 125.2, 132.2, 134.5, 146.2, 154.8, 159.2 and 165.3. δ_{H} (CDCl₃, 250 MHz): 0.90 (3H, t, J 7.2), 1.38 (4H, m), 1.77 (2H, t, J 7.2), 3.91 (3H, s), 3.94 (3H, s), 4.08 (2H, t, J 6.9), 6.86 (1H, t, J 9.0), 7.33 (1H, d, t 9.0), 8.5 (1H, s) and 9.08 (1H, br s).

### Synthesis Example 4-6

### 7-Methoxy-2-oxo-8-pentyloxy-1,2-dihydro-quinoline-3-carboxylic acid

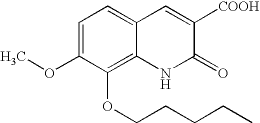

7-Methoxy-2-oxo-8-pentyloxy-1,2-dihydro-quinoline-3-carboxylic acid methyl ester (440 mg) was dissolved in methanol (13 ml) with heating. A solution of NaOH (220 mg) in water (5.5 ml) was added and the mixture stirred at 50° C. for 2 hours. The reaction was cooled in an ice bath and acidified with conc. HCl, giving a precipitate. The mixture was extracted with ethyl acetate and the organic phase dried over Na₂SO₄ and evaporated. Recrystallisation from ethanol gave the title compound as a white powder (300 mg). δ_{C} (CDCl₃, 62.9 MHz): 14.0, 22.4, 27.9, 29.8, 56.5, 74.0, 110.3, 114.4, 115.4, 126.0, 133.0, 133.6, 147.4, 155.5, 164.2 and 165.2. δ_{H} (CDCl₃, 250 MHz): 0.90 (3H, t, J 6.9), 1.39 (4H, m), 1.80 (2H, m), 4.0 (3H, s), 4.17 (2H, t, J 7.0), 7.02 (1H, d, J 8.9), 7.48 (1H, s, J 8.8), 8.84 (1H, s), 10.0 (1H, br s) and 14.0 (1H, br s).

### Synthesis Example 4-7

### 7-Methoxy-2-oxo-8-pentyloxy-1,2-dihydro-quinoline-3-carbonyl chorlide

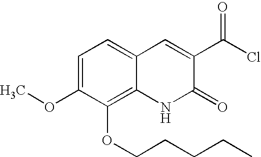

7-Methoxy-2-oxo-8-pentyloxy-1,2-dihydro-quinoline-3-carboxylic acid (0.27 g) was dissolved in DCM (5 ml). A catalytic quantity of DMF was added followed by thionyl chloride (0.5 ml). The solution was stirred at room temperature for 1 hour. Toluene (2×10 ml) was added and the solvent evaporated. Upon cooling, the title compound was obtained as a yellow crystalline solid.

### Synthesis Example 4-8

### 7-Methoxy-2-oxo-8-pentyloxy-1,2-dihydro-quinoline-3-carboxylic acid (benzo[1,3]dioxol-5-yl-methyl)-amide

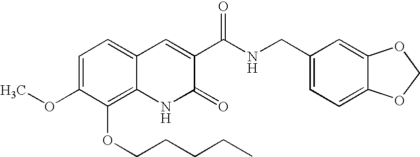

3,4-methylenedioxybenzylamine (1 ml) was dissolved in DCM (10 ml). To this was added dropwise a solution of 7-methoxy-2-oxo-8-pentyloxy-1,2-dihydro-quinoline-3-carbonyl chloride in DCM (10 ml). A white precipitate is produced and the mixture stirred at room temperature for 2 hours. The solvent was evaporated, the residue dissolved in chloroform and purified by column chromatography (CHCl₃:ethyl acetate, 10:1). Evaporation gave a clear oil which solidified overnight to give the title compound as a white solid. δ_{C} (CDCl₃, 62.9 MHz): 14.0, 22.5, 28.0, 29.9, 43.3, 56.3, 73.8, 101.0, 108.3, 108.4, 109.1, 114.3, 119.5, 120.9, 125.4, 132.4, 132.6, 133.5, 145.1, 146.7, 147.8, 154.4, 162.1 and 163.6. δ_{H} (CDCl₃, 250 MHz): 0.91 (3H, t, J 7.1), 1.38 (4H, m), 1.78 (2H, m), 3.95 (3H, s), 4.11 (2H, t, J 6.8), 4.55 (2H, d, J 5.5), 5.90 (2H, s), 6.7-6.9 (3H, m), 6.92 (1H, d, J 8.8), 7.43 (1H, d, J 8.8), 8.86 (1H, s), 9.19 (1H, br s) and 9.93 (1H, br s).

Biological Methods

MTT J774 Murine Macrophage Viability Assay

Cultures of the murine macrophage cell line J774 is an established model system with which to screen for activity of agents that influence osteoclast function (see, e.g., Luckman et al., 1998).

MTT (3-(4,5-dimethylthiazol-2-yl)-2,5-diphenyl tetrazolium bromide) has an orange colour and is soluble in the medium used for cell culture. The mitochondrial enzyme succinate dehydrogenase acts upon MTT in living cells to produce the insoluble purple coloured formazan. The amount of formazan produced, as measured by UV/visible spectroscopy, is proportional to the number of viable cells.

J774 cells were plated at 10⁴ cells per well in 150 μL αMEM in 96-well plates and grown overnight. The next day test compounds were added to the cultures, and cultures were continued for another 24-48 hours. At the end of the culture period, cell survival was determined using the tetrazolium dye-based MTT assay as previously described (see, e.g., MacPherson et al., 1999).

Briefly, MTT (5 mg/ml MTT in αMEM) was added to each well (1:10 v/v, 15 μL) and the cells incubated for 4 hours. The medium was carefully removed using a needle without dislodging the crystal layer. 100 μL acidified isopropanol (4 M HCl 1:100 v/v in isopropanol) was added to each well and the purple crystals allowed to dissolve. The absorbance was measured in a plate reader at 540 nm, with 690 nm as reference. The controls were a deep purple colour, indicating a high number of live cells. The results for each test compound were expressed as a % of the average control value.

In general, compounds studied were made up as 100 mM solutions in DMSO. These stock solutions were then diluted 100× in culture medium. From these 1 mM solutions, convenient quantities (3-15 μL) were added directly to the wells so as to give the desired final compound concentration.

IC50 values for individual agents were calculated using GraphPad Prism (GraphPad Software, San Diego) and were defined as the concentration of agent required to reduce cell survival to 50% of the control value at 72 hours

Alamar Blue J774 Murine Macrophage Viability Assay

J774 cells were plated at 10⁴ cells per well in 150 μL αMEM (α Modified Eagle Medium) in 96-well plates and grown overnight. The next day, compounds were added to the cultures, and culture was continued for another 72 hours. At the end of the culture period cell survival was determined using an Alamar Blue assay as previously described (see, e.g., Nociari et al., 1998).

Alamar Blue is an oxidation-reduction sensitive indicator. The dye itself is in the oxidised state, which is blue and non-fluorescent. The dye can accept electrons from reducing species, such as NADPH and FADH, to form a reduced dye species, which is red and fluorescent. Thus the transformation from oxidised form to reduced form can be measured by fluorimetric or colourimetric means. For fluorescence measurements, 530-560 nm excitation and 590 nm emission wavelengths are typically used. For colourimetric measurements, absorbance is measured at 570 nm (reduced form) and 600 nm (oxidised form) and a simple calculation performed to determine the relative quantities of the two species.

A high ratio of the reducing species, NADPH and FADH, to the corresponding oxidised species, NADP and FAD, is an indicator that cells are proliferating and viable. A low ratio indicates cells that are quiescent or non-viable.

Briefly, Alamar Blue (Biosource International) was added undiluted to the each well (1:10 v/v, 15 μL). The plate was incubated at 37° C. for 3-4 hours and the fluorescence was measured at 570 nm, with a 25 nm bandwidth. A high reading indicated cells with normal viability, and a low reading indicates cells that have been damaged and are no longer proliferating normally. The controls gave a high fluorescence reading, indicating a high number of live, healthy cells. A potent test compound gave a low fluorescence reading. The average results for each test compound (e.g., n=5) were expressed as a % of the average control value.

In general, compounds studied were made up as 100 mM solutions in DMSO. These stock solutions were then diluted 100 or 1000× in culture medium (αMEM). From these 1 mM or 100 μM solutions, convenient quantities (3-15 μL) were added directly to the wells so as to give the desired final compound concentration.

IC50 values for individual agents were calculated using GraphPad Prism (GraphPad Software, San Diego) and were defined as the concentration of agent required to reduce cell survival to 50% of the control value at 72 hours.

This assay offers numerous advantages over other assays, including MTT assays: it permits a higher throughput; it is more sensitive; it is non-damaging to the cells; it is faster; it generally gives an identical result to MTT assay.

Rabbit Osteoclast Culture

Osteoclast survival and activity was studied in cultures of rabbit osteoclasts. Osteoclasts were isolated from the long bones of 2-3 day-old rabbits as described previously (see, e.g., Coxon et al., 2000), plated on dentine slices and cultured in αMEM supplemented with 10% FCS and penicillin and streptomycin at 37° C. in 5% CO₂ for 48 hours in the presence or absence of test compounds. At the end of the culture, the osteoclasts were identified by staining for tartrate-resistant acid phosphatase (TRAcP) and resorption pit area was quantified by reflected light microscopy as described previously (see, e.g., van't Hof et al., 1997).

Bone Marrow Co-Culture Assay

Osteoclast formation and activity was studied using an adaptation (see, e.g., van't Hof et al., 1997) of the osteoblast-bone marrow co-culture assay originally described previously (see, e.g., Takahashi et al., 1988). This is another experimental model system to screen for osteoclast-inhibitor and osteoclast-stimulatory agents.

Osteoblasts were isolated from the calvarial bones of 2-day-old mice by sequential collagenase digestion (type I collagenase, Sigma) and cultured in αMEM supplemented with 10% FCS and penicillin and streptomycin at 37° C. in 5% CO₂. Bone marrow cell populations containing osteoclast precursors were isolated from the long bones of 3-5 month old mice and erythrocytes were removed by Ficoll Hypaque density gradient centrifugation. The resulting bone marrow cells were washed with PBS and resuspended in culture medium.

Osteoblasts and bone marrow cells were plated at 10⁴ cells/well and 2×10⁵ cells/well, respectively, in 96-well plates in 150 μL of αMEM supplemented with 10% FCS, antibiotics and 10 nM 1,25-dihydroxyvitamin D₃. Test substances were added on day 7 and the cultures were terminated on day 10. At the end of the culture period, osteoclasts were identified by TRAcP staining and resorption pits were quantified by reflected light microscopy, as described above.

Mouse Osteoblast Culture

Osteoblasts were isolated as described above and plated at 10⁴ cells/well in 96-well plates in 100 μL of αMEM supplemented with 10% FCS and antibiotics. Test substances were added after 24 hours and left for 72 hours. Cell viability was assessed using the Alamar Blue assay.

In Vivo Studies

Animals. Female 9 week-old C57/BL6 mice. Animals were housed in a designated animal facility and routinely maintained on a 12 h:12 h light:dark cycle and given ad libitum access to food and water.

Ovariectomy induced bone loss. Bilateral ovariectomy (Ovx) was performed under general anaesthesia. Sham ovariectomy (Sham) was similarly performed but with externalisation and replacement of the ovaries. Animals were given a daily injection of (a) candidate compound (e.g., 6 mg/kg) in vehicle (corn oil), or (b) vehicle (corn oil). After 21 days, the animals were killed, and the tibial bones were dissected and used for bone mineral density measurements and histomorphometric analysis (see below).

Bone Mineral Measurements. Measurements of bone mineral density (BMD) at the left proximal tibial metaphysis were determined by peripheral quantitative computed tomography (pQCT) using an XCT Research M bone densitometer with a voxel size of 70 μm and analysis software version 5.1.4. (Stratec Medizintechnik, Pforzheim, Germany). Daily quality assurance measurements were performed using a plexi-coated PVC-fluorinated hydrocarbon phantom according to the manufacturer's instructions.

Bone Histomorphometry. Histomorphometry was performed on left tibiae. The bones were dissected free of soft tissues, fixed in 4% buffered formalin/saline (pH 7.4) and embedded in methyl methacrylate. Longitudinal sections (4 μm) were then prepared and stained with Von Kossa and counterstained with Paragon. Histomorphometric measurements were made on sections of the proximal metaphysis distal to the epiphyseal growth plate at 20× magnification using a Zeiss Axioskop (Carl Zeiss, Welwyn Garden City, UK) coupled to an image analysis system running in-house designed software developed using Aphelion ActiveX Objects (Adcis SA, Hérouville-Saint-Clair, France). Bone histomorphometric variables were expressed according to the guidelines of the American Society of Bone and Mineral Research Nomenclature Committee (Eriksen, E. F., Axelrod, D. W., Melsen., F, 1994, Bone Histomorphometry, Raven Press, New York, USA).

Statistical Analyses. Statistical-analyses were performed using SPSS for Windows version 9. Significant differences between groups were determined by ANOVA followed by post-hoc testing using Dunnet's post-test. All data are presented as means±SEM unless stated otherwise. Values of p less than 0.05 were considered significant.

Dual Energy X-Ray Absorptiometry

Whole body, spine and femoral bone mineral density (BMD) and bone mineral content (BMC) were determined using a Piximus small animal DEXA scanner at the start and the end of the experiment. Daily quality assurance measurements were performed using a phantom according to the manufacturer's instructions.

### BIOLOGICAL EXAMPLES

The following examples are provided solely to illustrate the present invention and are not intended to limit the scope of the invention, as described herein.

### Biological Example 1

J774 murine macrophage viability studies were performed using the Alamar Blue assay, as described above, for the known cannabinoid receptors: CB1 inverse agonist AM251 and CB1/CB2 inverse agonist AM630.

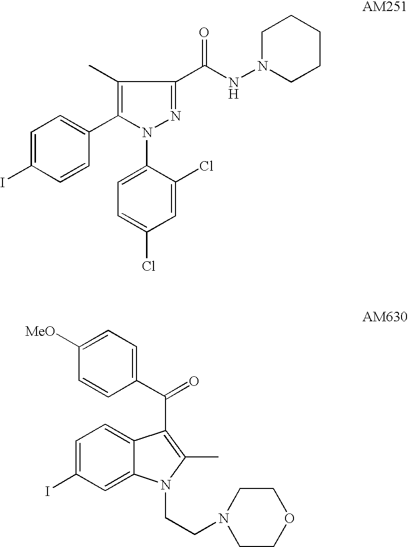

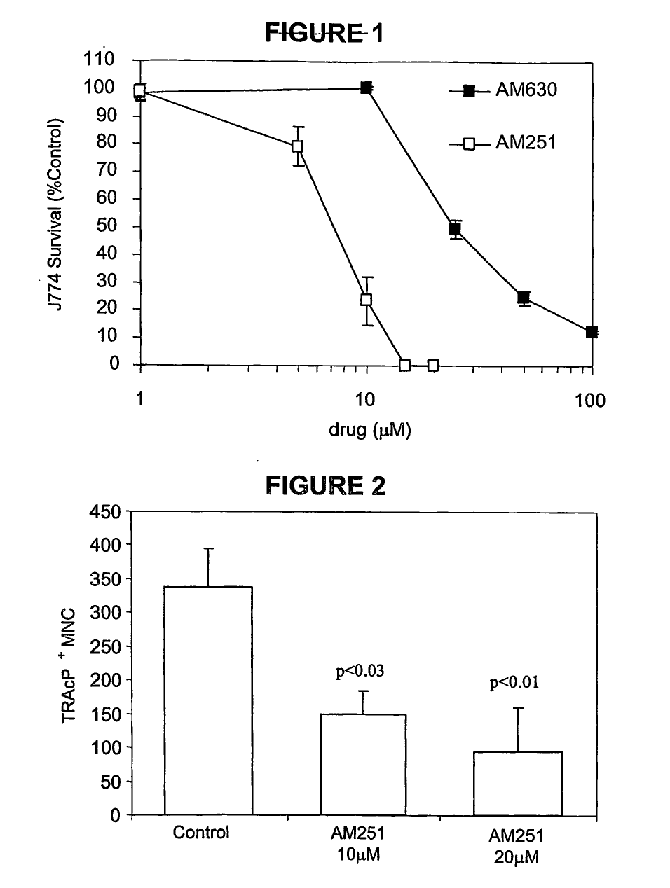

FIG. 1 is a graph showing J774 survival (% control) versus concentration of drug, for compounds AM630 (▪) and AM251 (□) after 72 hours, by Alamar Blue assay (n=3).

The known CB1 receptor inverse agonist, AM251, was highly potent as a modulator of J774 survival after 72 hours (shown) and both 24 and 48 hours (data not shown), and had an IC50 of about 5 μM.

Similarly, the known CB2 receptor inverse agonist, AM630, also modulated J774 survival after 72 hours-(shown) and both 24 and 48 hours (data not shown), with an IC50 of about 20 μM.

By comparison, the CB1/CB2 receptor agonists CP55940 and WIN 55212 did not affect J774 survival at concentrations of up to 100 μM (data not shown).

The known CB2 selective receptor agonist JWH133 did not affect J774 survival at concentrations of up to 20 μM (data not shown).

Also by comparison, the CB1 receptor agonist/CB2 receptor partial agonist anandamide (AEA) had no effect at low concentrations, but inhibited J774 survival at high concentrations with an IC50 of about 40 μM (data not shown).

This data demonstrates that CB receptor inverse agonists, such as AM251 and AM630 are highly active against macrophages and therefore have potential as potent osteoclast inhibitors.

### Biological Example 2

The effects of the known cannabinoid receptor CB1 inverse agonist AM251 on isolated rabbit osteoclasts was studied using the rabbit osteoclast culture, as described above.

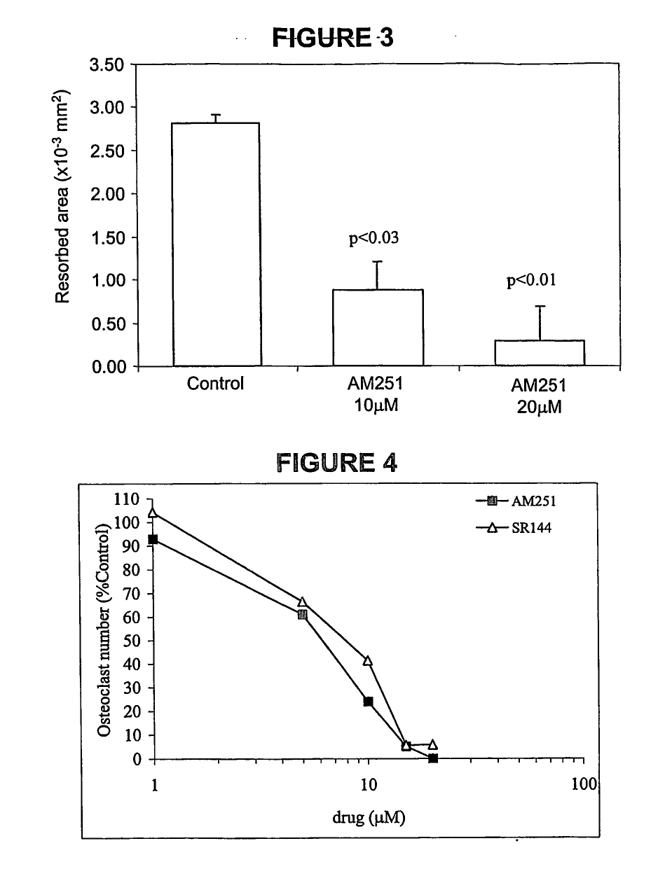

The results are shown in FIG. 2 and FIG. 3.

FIG. 2 is a bar-graph of TRAcP MNC for control, AM251 (at 10 μM) (p<0.03), and AM251 (at 20 μM) (p<0.01).

FIG. 3 is a bar-graph of resorbed area for control, AM251 (at 10 μM) (p<0.03), and AM251 (at 20 μM) (p<0.01).

AM251 inhibited survival of tartrate resistant acid phosphatase positive multinucleated osteoclasts (TRAcP MNC).

AM251 also reduced bone resorption at concentrations of 10 μM and 20 μM.

This data demonstrates that the results obtained in the macrophage system are a viable model for osteoclast function and that the CB1 inverse agonist, AM251, is highly potent against rabbit osteoclast survival and function

### Biological Example 3

The known cannabinoid receptor CB2 selective inverse agonist SR144528 and CB1 selective inverse agonist AM251, were studied in the osteoblast bone marrow co-culture assay, as described above.

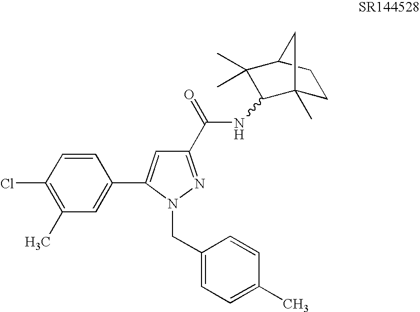

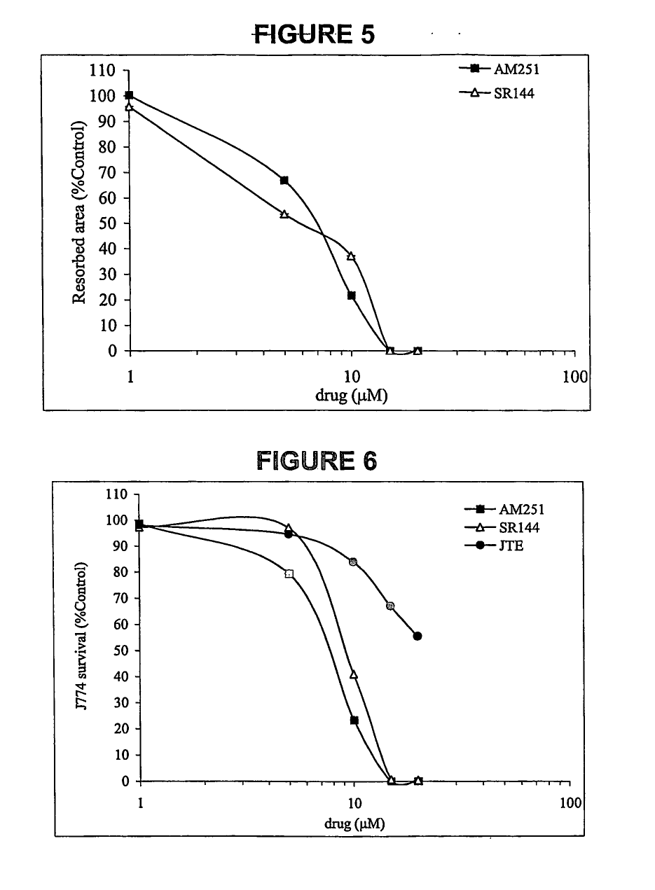

The osteoblast bone marrow co-culture assay data are shown in FIG. 4 and FIG. 5.

FIG. 4 is a graph of TRAcP MNC versus concentration of AM251 (▪) and SR144528 (“SR144”) (Δ) (n=3).

FIG. 5 is a graph of resorbed area versus concentration of AM251 (▪) and SR144528 (“SR144”) (Δ) (n=3).

AM251 and SR144528 significantly inhibited osteoclast function, and significantly inhibited bone resorption, with an IC50 of about 5 μM.

These results demonstrate that both the CB2 selective inverse agonist SR144528 and the CB1 selective inverse agonist AM251 are highly potent against osteoclast survival and function.

### Biological Example 4

The effects of known cannabinoid receptor ligands: CB2 inverse agonist SR144528; CB1 inverse agonist AM251; and CB2 inverse agonist JTE-907 were studied in the J774 murine macrophage system, assayed using Alamar Blue, as described above.

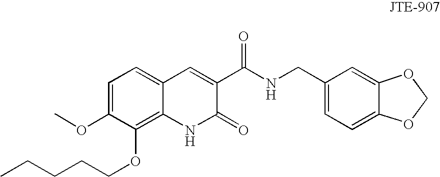

The Alamar Blue J774 murine macrophage viability assay data is shown in FIG. 6.

FIG. 6 is a graph of Alamar Blue J774 murine macrophage survival (% control) versus concentration of drug, for compounds AM251 (▪), SR144528 (“SR144”) (Δ) and JTE-907 (“JTE”) (●) (n=3).

SR144528, JTE-907 and AM251 significantly inhibited J774 survival, with an IC50 of about 9 μM, about 25 μM, and about 7 μM, respectively.

These results demonstrate that both CB1 and CB2 receptor inverse agonists, such as SR144528, JTE-907 and AM251 are highly potent against macrophages. There does not appear to be a relationship between selectivity and activity.

### Biological Example 5

The effects of the known cannabinoid receptor ligands: CB2 inverse agonist SR144528; CB1 inverse agonist AM251; and CB2 inverse agonist JTE-907, on isolated mouse osteoblasts was studied using the mouse osteoblast culture, as described above.

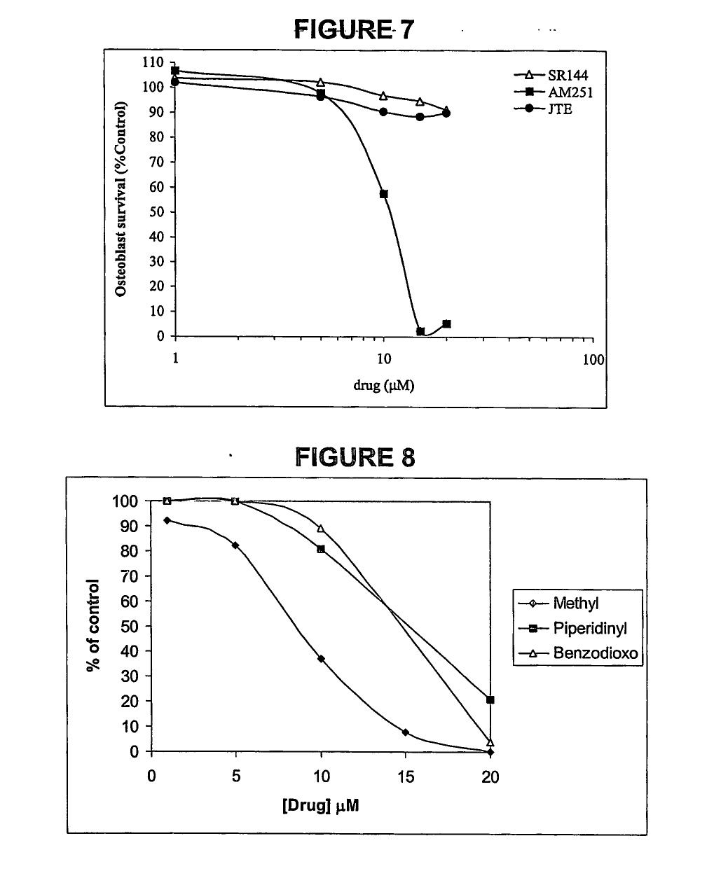

The mouse osteoblast culture data is shown in FIG. 7

FIG. 7 is a graph of osteoblast survival (% control) versus concentration of drug, for compounds SR144528 (“SR144”) (Δ), AM251 (▪) and JTE-907 (“JTE”) (●) (n=3).

These results demonstrate that the CB1 selective inverse agonist AM251 is highly potent against osteoblasts whereas the CB2 selective inverse agonists SR144528 and JTE-907 show little activity. This suggests that CB2 selectivity may be important in the development of a suitable drug for treating bone loss.

### Biological Example 6

The effects of compounds related to SR144528 were further investigated using several SR144528 analogs of unknown biological function:

(a) the SR144528 precursor: 5-(4-Chloro-3-methyl-phenyl)-1-(4-methyl-benzyl)-1H-pyrazole-3-carboxylic acid methyl ester (“methyl”);

(b) a compound bearing the amine used in AM251 and the remainder derived from SR144528: 5-(4-Chloro-3-methyl-phenyl)-1-(4-methyl-benzyl)-1H-pyrazole-3-carboxylic acid piperidin-1-yl amide (“piperidinyl”);

(c) a compound bearing the amine used in JTE-907 and the remainder derived from SR144528: 5-(4-Chloro-3-methyl-phenyl)-1-(4-methyl-benzyl)-1H-pyrazole-3-carboxylic acid (benzo[1,3]dioxol-5-yl-methyl) amide (“benzodioxo”).

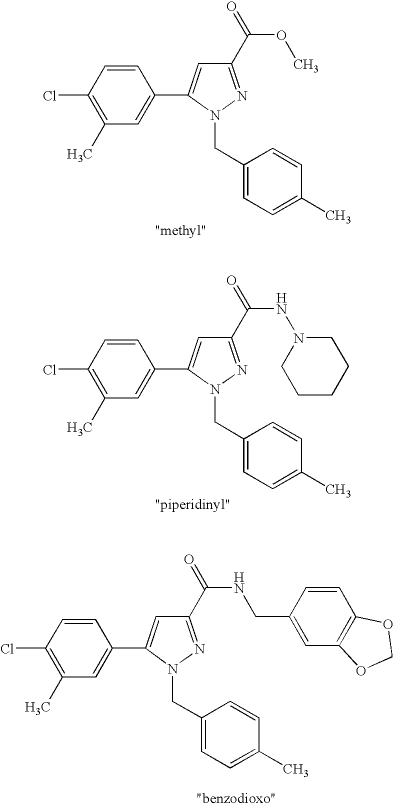

These analogs were studied using the Alamar Blue J774 murine macrophage viability assay, described above.

The viability assay data is shown in FIG. 8.

FIG. 8 is a graph of Alamar Blue J774 murine macrophage survival (% control) versus concentration of drug, for three analogs of SR144528: “methyl” (♦), “piperidinyl” (▪) (n=3), and “benzodioxo” (Δ).

The three analogs (“methyl”, “piperidinyl”, and “benzodioxo”) significantly inhibited J774 survival, with IC₅₀ values of about 9 μM, about 15 μM, and about 15 μM, respectively.

These results demonstrate that derivatives related to SR144528 are highly potent against macrophages, most notably the methyl ester. This demonstrates that both esters and amides having the SR144528 skeleton are useful as highly potent drugs against macrophages and related cells.

### Biological Example 7

Compound AM251 (dosage, 6 mg/kg) was investigated in vivo using ovariectomy induced bone loss, assessed using peripheral quantitative computed tomography, as described above.

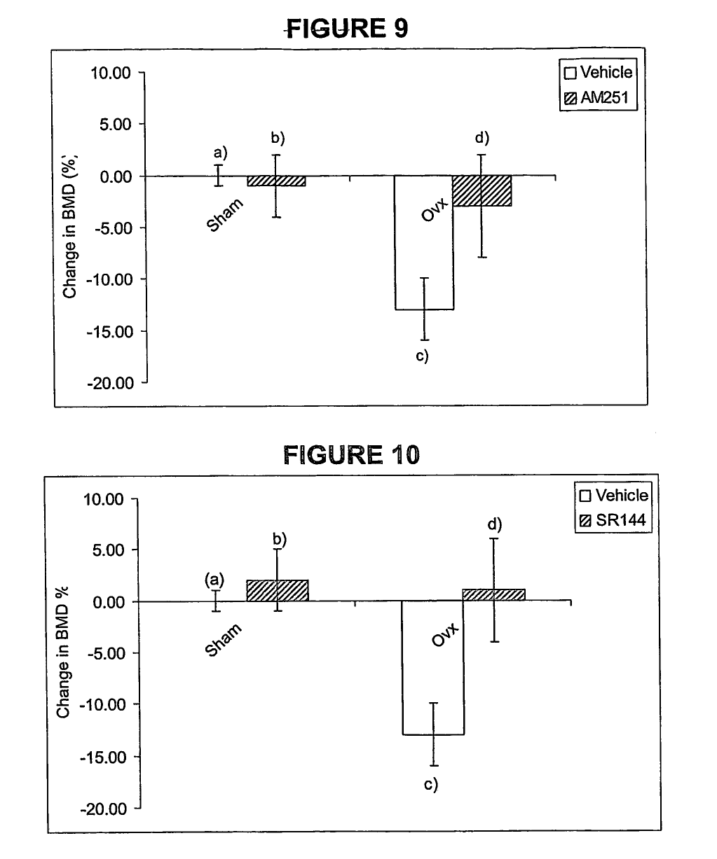

The ovariectomy-induced bone loss data are shown in FIG. 9.

FIG. 9 is a bar graph showing percent changes in trabecular density, for: (a) Sham operation, no drug; (b) Sham operation, AM251 (6 mg/kg); (c) OVX operation, no drug; and (d) OVX operation, AM251 (6 mg/kg).

The data demonstrates the ability of AM251 to reverse the effects of ovariectomy-induced bone loss, and show that AM251 is very effective at reversing the bone loss seen in this model for post-menopausal osteoporosis.

### Biological Example 8

Compound SR144528 (dosage, 6 mg/kg) was investigated in vivo using ovariectomy induced bone loss, assessed using peripheral quantitative computed tomography, as described above.

The ovariectomy-induced bone loss data are shown in FIG. 10.

FIG. 10 is a bar graph showing percent changes in trabecular density, for: (a) Sham operation, no drug; (b) Sham operation, SR144528 (“SR144”) (6 mg/kg); (c) OVX operation, no drug; and (d) OVX operation, SR144528 (“SR144”) (6 mg/kg).

The data demonstrates the ability of SR144528 to reverse the effects of ovariectomy-induced bone loss, and shows that SR144528 is very effective at reversing the bone loss seen in this model for post-menopausal osteoporosis.

### Biological Example 9

Compound SR144528 (dosage, 6 mg/kg) was investigated in vivo using ovariectomy induced bone loss, assessed using dual energy x-ray absorptiometry as described above.

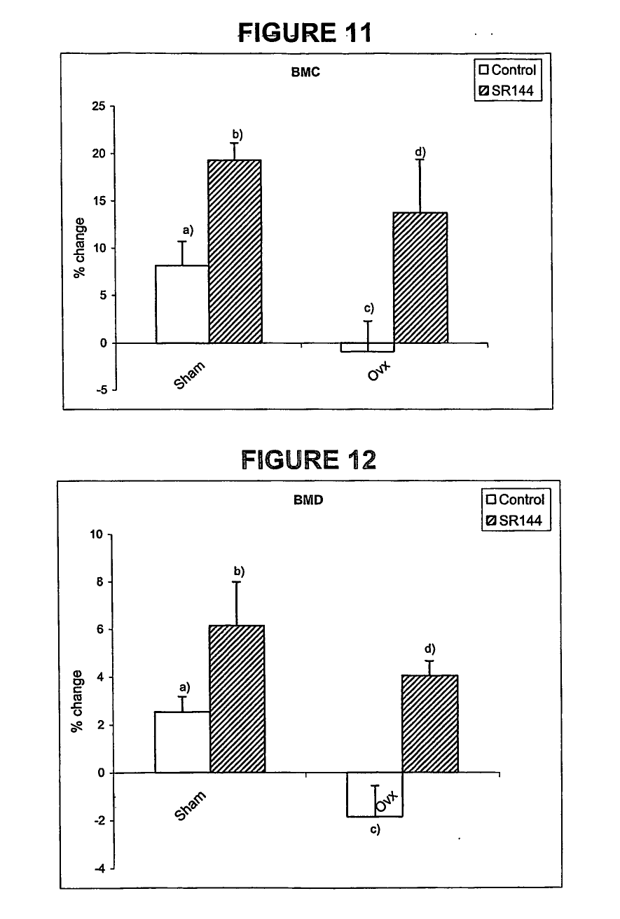

The ovariectomy-induced bone loss data are shown in FIGS. 11 and 12.

FIG. 11 is a bar graph showing percent changes in femoral bone mineral content, for: (a) Sham operation, no drug; (b) Sham operation, SR144528 (“SR144”) (6 mg/kg); (c) OVX operation, no drug; and (d) OVX operation, SR144528 (“SR144”) (6 mg/kg) as measured by dual energy x-ray absorptiometry.

FIG. 12 is a bar graph showing percent changes in femoral bone mineral density for (a) Sham operation, no drug; (b) Sham operation, SR144528 (“SR144”) (6 mg/kg); (c) OVX operation, no drug; and (d) OVX operation, SR144528 (“SR144”) (6 mg/kg) as measured by dual energy x-ray absorptiometry.

The data demonstrates the ability of SR144528 to reverse the effects of ovariectomy-induced bone loss at the femoral site, and shows that SR144528 is very effective at reversing the bone loss seen in this model for post-menopausal osteoporosis. Furthermore, it shows that SR144528 increases femoral BMC and BMD in a healthy subject, relative to control.

All of the above results demonstrate that both CB1 and CB2 inverse agonists are highly potent against osteoclasts and related cell types and have excellent potential in the treatment of osteoporosis and other bone disorders. In addition, compounds having a strong CB2 selectivity show little effect on osteoblasts, suggesting that CB2 inverse agonists, in particular, have excellent potential in the treatment of osteoporosis and other bone disorders.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention as described herein.

### REFERENCES

A number of patents and publications are cited herein in order to more fully describe and disclose the invention and the state of the art to which the invention pertains. Full citations for these references are provided herein. Each of these references is incorporated herein by reference in its entirety into the present disclosure.
Anantanarayan et al., 1999, “3(5)-heteroaryl substituted pyrazoles as p38 kinase inhibitors,” U.S. Pat. No. 5,932,576, granted Aug. 3, 1999.
Anantanarayan et al., 2002, “3(5)-heteroaryl substituted pyrazoles as p38 kinase inhibitors,” U.S. Pat. No. 6,335,336, granted Jan. 1, 2002.
Anantanarayan et al., 2003a, “3(5)-heteroaryl substituted pyrazoles as p38 kinase inhibitors,” U.S. Pat. No. 6,579,873, granted Jun. 17, 2003.
Anantanarayan et al., 2003b, “Substituted pyrazoles as p38 kinase inhibitors,” U.S. Pat. No. 6,525,059, granted Feb. 25, 2003.
Baldock P A, Sainsbury A, Couzens M, Enriquez R F, Thomas G P, Gardiner E M, Herzog H, 2002, “Hypothalamic Y2 receptors regulate bone formation,” J. Clin. Invest., Vol. 109, pp. 915-921.
Barth, F., et al., 1999, “3-pyrazolecarboxamide derivatives having cannabinoid receptor affinity,” U.S. Pat. No. 5,925,768 granted 20 Jul. 1999.
Barth, F., et al., 2002a, “3-Arylindole Derivatives and Their Use as CB2 Receptor Agonists,” published international (PCT) patent publication number WO 02/42269 published 30 May 2002.
Barth, F., et al., 2002b, “Pyrazolecarboxylic acid derivatives, their preparation, pharmaceutical compositions containing them”, U.S. Pat. No. 6,432,984 granted 13 Aug. 2002.
Barth, F., et al., 2003a, “Terphenyl derivatives, preparation thereof, compositions containing same,” published international patent application publication number WO 03/084943, published 16 Oct. 2003.
Barth, F., et al., 2003b, “Indole derivatives, method for preparing same and pharmaceutical compositions containing same,” published international patent application publication number WO 03/097597, published 27 Nov. 2003.
Bass, C E, et al., 2002, “SR-141716A-Induced Stimulation of Locomotor Activity: A Structure-Activity Relationship Study,” Pharmacology, Biochemistry and Behavior, Vol. 74, pp. 31-40.
Bender, P. E. et al., 1999, “Novel Cannabinoid Receptor Modulators,” international (PCT) patent publication number WO 99/26612 published 3 Jun. 1999.
Bender, P. E., et al., 1998, “Novel Cannabinoid Receptor Agonists,” international (PCT) patent publication number WO 98/41519 published 24 Sep. 1998.
Bidaut-Russell, M., Devane, W. A. and Howlett, A. C., 1990, “Cannabinoid receptors and modulation of cyclic AMP accumulation in the rat brain,” J. Neurochem., Vol. 55, pp. 21-26.
Blake et al., 1997, “Prevention of bone resorption,” U.S. Pat. No. 5,674,887, granted Oct. 7, 1997.
Bouaboula M, Desnoyer N, Carayon P, Combes T, Casellas P, 1999, “Gi protein modulation induced by a selective inverse agonist for the peripheral cannabinoid receptor CB2: implication for intracellular signalization cross-regulation,” Mol. Pharmacol., Vol. 55, pp. 473-480.
Bouaboula M, Perrachon S, Milligan L, Canat X, Rinaldi-Carmona M, Portier M, Barth F, Calandra B, Pecceu F, Lupker J, Maffrand J P, Le Fur G, Casellas P, 1997, “A selective inverse agonist for central cannabinoid receptor inhibits mitogen-activated protein kinase activation stimulated by insulin or insulin-like growth factor 1. Evidence for a new model of receptor/ligand interactions,” J. Biol. Chem., Vol. 272, pp. 22330-22339.
Bouaboula M, Poinot-Chazel C, Marchand J, Canat X, Bourrie B, Rinaldi-Carmona M, Calandra B, Le Fur G, Casellas P, 1996, “Signaling pathway associated with stimulation of CB2 peripheral cannabinoid receptor. Involvement of both mitogen-activated protein kinase and induction of Krox-24 expression,” Eur. J. Biochem., Vol. 237, pp. 704-711.
Breivogel C S, Griffin G, Di M, V, Martin B R, 2001, “Evidence for a new G protein-coupled cannabinoid receptor in mouse brain,” Mol. Pharmacol., Vol. 60, pp. 155-163.
Calignano A, La Rana G, Giuffrida A, Piomelli D, 1998, “Control of pain initiation by endogenous cannabinoids,” Nature, Vol. 394, pp. 277-281.
Chackalamannil et al., 2001, “Thrombin Receptor Antagonists,” international (PCT) patent publication number WO 01/96330 published 20 Dec. 2001.

Chambers T J, Magnus C J, 1982, “Calcitonin alters the behaviour of isolated osteoclasts,” J. Pathol., Vol. 136, pp. 2740.

Cheng Y, Prusoff W H, 1973, “Relationship between the inhibition constant (K1) and the concentration of inhibitor which causes 50 percent inhibition (150) of an enzymatic reaction,” Biochem. Pharmacol., Vol. 22, pp. 3099-3108.

Chesnut C H, Silverman S, Andriano K, Genant H, Gimona A, Harris S, Kiel D, LeBoff M, Maricic M, Miller P, Moniz C, Peacock M, Richardson P, Watts N, Baylink D, 2000, “A randomized trial of nasal spray salmon calcitonin in postmenopausal women with established osteoporosis: the prevent recurrence of osteoporotic fractures study,” Am. J. Med., Vol. 109, pp. 267-276.
Childers, S. R. and Deadwyler, S. A., 1996, “Role of cyclic AMP in the actions of cannabinoid receptors,” Biochemical Pharmacology, Vol. 52, pp. 819-827.
Clayton, N., Marshall, F. H., Bountra, C., O'Shaughnessy, C. T., 2002, “CB1 and CB2 cannabinoid receptors are implicated in inflammatory pain,” Pain, Vol. 96, pp. 253-260.
Compston J E, Vedi S, Croucher P I, Garrahan N J, and O'Sullivan M M. 1994, “Bone turnover in non-steroid treated rheumatoid arthritis,” Annals of the Rheumatic Diseases, Vol. 53, No. 3, pp. 163-166.
Conti, S., Costa, B., Colleoni, M., Parolaro, D., and Giagoni, G., 2002, “Antiinflammatory action of endocannabinoid palmitoylethanolamide and the synthetic cannabinoid nabilone in a model of acute inflammation in the rat,” Brit. J. Pharmacol., Vol. 135, pp. 181-187.
Coxon F P, Helfrich M H, Van't Hof R, Sebti S, Ralston S H, Hamilton A, Rogers M J, 2000, “Protein geranylgeranylation is required for osteoclast formation, function, and survival: inhibition by bisphosphonates and GGTI-298,” J. Bone Miner. Res., Vol. 15, pp. 1467-1476.
Craib S J, Ellington H C, Pertwee R G, Ross R A, 2001, “A possible role of lipoxygenase in the activation of vanilloid receptors by anandamide in the guinea-pig bronchus,” Br. J. Pharmacol., Vol. 134, pp. 30-37.
Croucher P I, Vedi S, Motley R J, Garrahan N J, Stanton M R, and Compston J E, 1993, “Reduced bone formation in patients with osteoporosis associated with inflammatory bowel disease,” Osteoporosis. Int., Vol. 3, No. 5, pp. 236-241.
Di M, V, Bisogno T, Melck D, Ross R, Brockie H, Stevenson L, Pertwee R, De Petrocellis L, 1998, “Interactions between synthetic vanilloids and the endogenous cannabinoid system,” FEBS Lett., Vol. 436, pp. 449-454.
Di M, V, De Petrocellis L, Fezza F, Ligresti A, Bisogno T, 2002, “Anandamide receptors,” Prostaglandins Leukot. Essent. Fatty Acids, Vol. 66, pp. 377-391.
Di M, V, De Petrocellis L, Sepe N, Buono A, 1996, “Biosynthesis of anandamide and related acylethanolamides in mouse J774 macrophages and N18 neuroblastoma cells,” Biochem. J., Vol. 316, pp. 977-984.
Didierlaurent et al., 1999, “Acid pyrazole derivatives, preparation method therefor, use thereof as drugs, novel use therefor, and pharmaceutical compositions containing such derivatives,” U.S. Pat. No. 5,942,622, granted Aug. 24, 1999.
Dragovich et al., 2003, “Antipicornaviral compounds and compositions, their pharmaceutical uses, and materials for their synthesis,” U.S. Pat. No. 6,514,997, granted Feb. 4, 2003.
Ducy P, Amling M, Takeda S, Priemel M, Schilling A F, Beil F T, Shen J, Vinson C, Rueger J M, Karsenty G, 2000, “Leptin inhibits bone formation through a hypothalamic relay: a central control of bone mass,” Cell, Vol. 100, pp. 197-207.
Dunford J E, Thompson K, Coxon F P, Luckman S P, Hahn F M, Poulter C D, Ebetino F H, Rogers M J, 2001, “Structure-activity relationships for inhibition of farnesyl diphosphate synthase in vitro and inhibition of bone resorption in vivo by nitrogen-containing bisphosphonates,” J. Pharmacol. Exp. Ther., Vol. 296, pp. 235-242.
Felder, C C, et al., 1998, “LY320135, a Novel Cannabinoid CB1 Receptor Antagonist, Unmasks Coupling of the CB1 Receptor to Stimulation of cAMP Accumulation,” Journal of Pharmacology and Experimental Therapeutics, Vol. 284, pp. 291-297.
Finke et al., 2003, “Substituted 2,3-diphenyl pyridines,” published international patent application publication number WO 03/082191, published 9 Oct. 2003.
Friary, R. J., et al., 2003, “Cannabinoid Receptor Ligands,” International (PCT) Patent Application publication number WO 03/042174 A1, published 22 May 2003.
Frith J C, Monkkonen J, Auriola S, Monkkonen H, Rogers M J, 2001, “The molecular mechanism of action of the antiresorptive and antiinflammatory drug clodronate: evidence for the formation in vivo of a metabolite that inhibits bone resorption and causes osteoclast and macrophage apoptosis,” Arthritis Rheum., Vol. 44, pp. 2201-2210.
Gallant et al., 1996, “New class of potent ligands for the human peripheral cannabinoid receptor,” Bioorg. Med. Chem. Lett., Vol. 6, pp. 2263.
Gareau, Y, et al., 1996, “Structure Activity Relationships of Tetrahydrocannabinol Analogues on Human Cannabinoid Receptors,” Bioorganic & Medicinal Chemistry Letters, Vol. 6, No. 2, pp. 189-194.
Garzon et al., 2003, “Bicyclic CB₂ cannabinoid receptor ligands,” published international patent application publication number WO 03/064359, published 7 Aug. 2003.
Gifford, A N, et al., 1997, “Effect of the Cannabinoid Receptor SPECT agent, AM 281, on Hippocampal Acetycholine Release from Rat Brain Slices,” Neuroscience Letters, Vol. 238, pp. 84-86.
Goutopoulos, A, Makriyannis, A., 2002, “From cannabis to cannabinergics: new therapeutic opportunities,” Pharmacology & Therapeutics, Vol. 95, pp. 103-117.
Green et al., 2003, “Pyrazole compositions useful as inhibitors of ERK,” U.S. Pat. No. 6,593,357, granted Jul. 15, 2003.
Guise, T A & Mundy G R, 1998, “Cancer and Bone”, Endocrine Reviews, Vol. 19, pp. 18-54.

Hagmann et al., 2003a, “Substituted imidazoles as cannabinoid receptor modulators,” published international patent application publication number WO 03/063781, published 7 Aug. 2003
Hagmann et al., 2003b, “Substituted aryl amides,” published international patent application publication number WO 03/087037, published 23 Oct. 2003.

Hale et al., 2003, “Pyrazole compositions useful as inhibitors of ERK,” U.S. Pat. No. 6,528,509, granted Mar. 4, 2003.
Hanson et al., 2000, “Pyrazole derivatives as p38 kinase inhibitors,” U.S. Pat. No. 6,087,381, granted Jul. 11, 2000.
Hanson et al., 2002, “Radio frequency ablation apparatus with remotely articulating and self-locking electrode wand,” U.S. Pat. No. 6,423,059, granted Jul. 23, 2002.
Hanson et al., 2003, “Pyrazole derivatives as p38 kinase inhibitors,” U.S. Pat. No. 6,503,930, granted Jan. 7, 2003.
Hanus L, Breuer A, Tchilibon S, Shiloah S, Goldenberg D, Horowitz M, Pertwee R G, Ross R A, Mechoulam R, Fride E, 1999, “HU-308: a specific agonist for CB(2), a peripheral cannabinoid receptor,” Proc. Natl. Acad. Sci. U.S.A., Vol. 96, pp. 14228-14233.
Hanus, L, et al., 1999, “HU-308: A Specific Agonist for CB2, a Peripheral Cannabinoid Receptor,” Proceedings of the National Academy of Science, Vol. 96, No. 25, pp. 14228-14223.
Huebner et al., 2001, “Estrogen receptor modulators,” U.S. Pat. No. 6,291,505, granted Sep. 18, 2001.
Huffman, J W, 2000, “The Search for Selective Ligands for the CB2 Receptor,” Current Pharmaceutical Design, Vol. 6, pp. 1323-1337.
Huffman, J W, et al., 1999, “3-(1′,1′-Dimethylbutyl)-1-deoxy-⁸Δ-THC and Related Compounds: Synthesis of Selective Ligands for the CB2 Receptor,” Bioorganic & Medicinal Chemistry, Vol. 7, pp. 2905-2914.
Huffman, J W, et al., 2001, “A Pyridone Analogue of Traditional Cannabinoids. A New Class of Selective Ligands for the CB2 Receptor,” Bioorganic & Medicinal Chemistry, Vol. 9, pp. 2863-2870.
Hughes, D E, Boyce, B F, 1997, “Apoptosis in bone physiology and disease,” Molecular Pathology, Vol. 50, pp. 132-137.
Inaba, T., et al., 2001, “2-Oxoquinoline Compounds and Medicinal Uses Thereof,” published European Patent Application EP 1 142 877 published 10 Oct. 2001.
Inaba, T., et al., 2003, “2-Oxoquinoline compounds and medicinal uses thereof,” U.S. Pat. No. 6,509,352 granted 21 Jan. 2003.
Iwamura, H, et al., 2001, “In Vitro and In Vivo Pharmacological Characterization of JTE-907, a Novel Selective Ligand for Cannabinoid CB2 Receptor,” Journal of Pharmacology and Experimental Therapeutics, Vol. 296, No. 2, pp. 420-425.
Kapadia et al., 2002, “Bis pyrazole-1H-pyrazole intermediates and their synthesis,” U.S. Pat. No. 6,492,529, granted Dec. 10, 2002.
Khanolkar et al., 2001, “Peripheral cannabinoid receptor (CB₂) selective ligands,” published international patent application publication number WO 01/28329, published 26 Apr. 2001.
Kolasa et al., 2002, “Sulfonylphenylpyrazole compounds useful as COX-2 inhibitors,” U.S. Pat. No. 6,472,416, granted Oct. 29, 2002.
Kong, Y Y, Yoshida, H, Sarosi, I, Tan, H L, Timms, E, Capparelli, C, et al., 1999, “OPGL is a key regulator of osteoclastogenesis, lymphocyte development and lymph-node organogenesis,” Nature, Vol. 397, pp. 315-323.
Kozak K R, Marnett L J, 2002, “Oxidative metabolism of endocannabinoids,” Prostaglandins Leukot. Essent. Fatty Acids, Vol. 66, pp. 211-220.
Kozlowski, J. A., et al., 2002, “Cannabinoid Receptor Ligands,” international (PCT) patent publication number WO 02/062750 published 15 Aug. 2002.
Kozlowski, J. A., et al., 2003, “Cannabinoid Receptor Ligands,” published US patent application number US 2003/0096844 A1, published 22 May 2003.
Krishnamurthy, M., Li, W., Moore, B. M., 2004, “Synthesis, biological evaluation, and structural studies on N1 and C5 substituted cycloalkyl analogues of the pyrazole class of CB1 and CB2 ligands,” Bioorganic and Medicinal Chemistry, Vol. 12, pp. 393-404.
Kruse et al., 2003a, “Novel 4,5-dihydro-1H-pyrazole derivatives having CB₁-antagonistic activity,” published international patent application publication number WO 03/026647, published 3 Apr. 2003.
Kruse et al., 2003b, “1H-Imidazole derivatives having CB₁ agonistic, CB₁ partial agonistic or CB₁-antagonistic activity,” published international patent application publication number WO 03/027076, published 3 Apr. 2003.
Lan, R, et al., 1999, “Structure-Activity Relationships of Pyrazole Derivatives as Cannabinoid Receptor Antagonists,” J. Med. Chem., Vol. 42, pp. 769-776.
Lange et al., 2003, “Thiazole derivatives having CB₁-antagonistic, agonistic or partial agonistic activity,” published international patent application publication number WO 03/078413, published 25 Sep. 2003.
Lichtman A H, Varvel S A, Martin B R, 2002, “Endocannabinoids in cognition and dependence,” Prostaglandins Leukot. Essent. Fatty Acids, Vol. 66, pp. 269-285.
Luckman S P, Coxon F P, Ebetino F H, Russell R G, Rogers M J, 1998, Heterocycle-containing bisphosphonates cause apoptosis and inhibit bone resorption by preventing protein prenylation: evidence from structure-activity relationships in J774 macrophages,” J. Bone Miner. Res., Vol. 13, pp. 1668-1678.
MacLennan S J, Reynen P H, Kwan J, Bonhaus D W, 1998, “Evidence for inverse agonism of SR141716A at human recombinant cannabinoid CB1 and CB2 receptors,” Br. J. Pharmacol., Vol. 124, pp. 619-622.
MacPherson H, Noble B S, Ralston S H, 1999, “Expression and functional role of nitric oxide synthase isoforms in human osteoblast-like cells,” Bone, Vol. 24, pp. 179-185.
Makriyannis, A., et al., 2003a, “Novel Pyrazole Analogs Acting on Cannabinoid Receptors,” published international (PCT) patent application publication no. WO 03/020217 published 13 Mar. 2003.
Makriyannis, A., et al., 2003b, “Heteroindanes: A New Class of Potent Cannibimimetic Ligands,” international (PCT) Patent Application publication number WO 03/035005 A2, published 1 May 2003.
Martin et al., 2003a, “Pyrazole cannabinoid agonist and antagonists,” U.S. Pat. No. 6,509,367 granted 21 Jan. 2003.
Martin et al., 2003b, “Cannabinoids,” published international patent application publication number WO 03/091189 (0-1797), published 6 Nov. 2003
Melck et al., 2000, “Suppression of nerve growth factor Trk receptors and prolactin receptors by endocannabinoids leads to inhibition of human breast and prostate cancer cell proliferation,” Endocrinol., Vol. 141, pp. 118.
Mittendorf, J., et al., 2002, “Aryl Sulfonamides and Analogues,” U.S. Pat. No. 6,469,054 B1 published 22 Oct. 2002.
Mundy, G R, 1996, Bone Remodelling and its disorders (2nd edition), London: Martin Dunitz.
Mussinu, J-M, et al., 2003, “Tricyclic Pyrazoles. Part 1: Synthesis and Biological Evaluation of Novel 1,4-Dihydroindeno[1,2-c]pyrazol-based Ligands for CB1 and CB2 Cannabinoid Receptors,” Bioorganic & Medicinal Chemistry, Vol. 11, pp. 251-263.
Nociari, M. N., et al., 1998, “A Novel one-step, highly sensitive fluorimetric assay to evaluate cell-mediated cytotoxicity,” Journal of Immunological Methods, Vol. 213, pp. 157-167.
Pacifici R, 1996, “Estrogen, cytokines, and pathogenesis of postmenopausal osteoporosis,” J. Bone Miner. Res., Vol. 11, pp. 1043-1051
Parolaro D, Massi P, Rubino T, Monti E, 2002, “Endocannabinoids in the immune system and cancer,” Prostaglandins Leukot. Essent. Fatty Acids, Vol. 66, pp. 319-332.
Pertwee R G, Ross R A, 2002, “Cannabinoid receptors and their ligands,” Prostaglandins Leukot. Essent. Fatty Acids, Vol. 66, pp. 101-121.
Pertwee, R G, 2001, “Cannabinoid Receptor Ligands,” Tocris Reviews, No. 16, April 2001 (publisher Tocris Cookson Inc., USA) (see also http://www.tocris.com, Technical Support and Resources, Life Science Reviews, Cannabinoid Receptor Ligands).
Raisz, L G, 1988, “Local and systemic factors in the pathogenesis of osteoporosis,” N. Engl. J. Med., Vol. 318, pp. 818-828.
Ralston, S H, 1997, “Science, Medicine and the Future: Osteoporosis,” Br. Med. J., Vol. 315, pp. 469472.
Rice A S, Farquhar-Smith W P, Nagy I, 2002, “Endocannabinoids and pain: spinal and peripheral analgesia in inflammation and neuropathy,” Prostaglandins Leukot. Essent. Fatty Acids, Vol. 66, pp. 243-256.
Rodan G A, Martin T J, 2000, “Therapeutic approaches to bone diseases,” Science, Vol. 289, pp. 1508-1514.
Rodan, G A, Harada, S, 1997, “The missing bone,” Cell, Vol. 89, pp. 677-680.
Rogers M J, Chilton K M, Coxon F P, Lawry J, Smith M O, Suri S, Russell R G, 1996, “Bisphosphonates induce apoptosis in mouse macrophage-like cells in vitro by a nitric oxide-independent mechanism,” J. Bone Miner. Res., Vol. 11, pp. 1482-1491.
Rogers M J, Frith J C, Luckman S P, Coxon F P, Benford H L, Monkkonen J, Auriola S, Chilton K M, Russell R G, 1999, “Molecular mechanisms of action of bisphosphonates,” Bone, Vol. 24, pp. 73S-79S.
Ross R A, Brockie H C, Stevenson L A, Murphy V L, Templeton F, Makriyannis A, Pertwee R G, 1999a, “Agonist-inverse agonist characterization at CB1 and CB2 cannabinoid receptors of L759633, L759656, and AM630,” Br. J. Pharmacol., Vol. 126, pp. 665-672.
Ross R A, Gibson T M, Brockie H C, Leslie M, Pashmi G, Craib S J, Di M, V, Pertwee R G, 2001, “Structure-activity relationship for the endogenous cannabinoid, anandamide, and certain of its analogues at vanilloid receptors in transfected cells and vas deferens,” Br. J. Pharmacol., Vol. 132, pp. 631-640.
Ross R A, Gibson T M, Stevenson L A, Saha B, Crocker P, Razdan R K, Pertwee R G, 1999b, “Structural determinants of the partial agonist-inverse agonist properties of 6′-azidohex-2′-yne-deltaβ-tetrahydrocannabinol at cannabinoid receptors,” Br. J. Pharmacol., Vol. 128, pp. 735-743.
Ross, R A, et al., 1999a, “Agonist-Inverse Agonist Characterization at CB1 and CB2 Cannabinoid Receptors of L759633, L759656 and AM630,” British Journal of Pharmacology, Vol. 126, pp. 665-672.
Ruiu, S., et al., 2003, “Synthesis and Characterization of NESS 0327: A Novel Putative Antagonist of the CB, Cannabinoid Receptor,” Journal of Pharmacology and Experimental Therapeutics, Vol. 306, pp. 363-370.
Sambrook P N and Reeve J, 1988, “Bone disease in rheumatoid arthritis,” Clin. Sci., Vol. 74, pp. 225-230.
Shim, J-Y, et al., 2002, “Molecular Interaction of the Antagonist N-(Piperidin-1-yl)-5-(4-Chlorophenyl)-1-(2,4-Dichlorophenyl)-4-Methyl-1H-Pyrazole-3-Carboxamide with the CB1 Cannabinoid Receptor,” J. Med. Chem., Vol. 45, pp. 1447-1459.
Smith, S. R., Denhart, G., Terminelli, C., 2001, “The anti-inflammatory activities of cannabinoid receptor ligands in mouse peritonitis models,” Eur. J. Pharmacol., Vol. 432, pp. 107-119.
Takahashi N, Akatsu T, Udagawa N, Sasaki T, Yamaguchi A, Moseley J M, Martin T J, Suda T, 1988, “Osteoblastic cells are involved in osteoclast formation,” Endocrinology, Vol. 123, pp. 2600-2602.
van't Hof R J, Ralston S H, 1997, “Cytokine-induced nitric oxide inhibits bone resorption by inducing apoptosis of osteoclast progenitors and suppressing osteoclast activity, J. Bone. Miner. Res., Vol. 12, pp. 1797-1804.
Vaulquelin et al., 2002, “New Insights into Insurmountable Antagonism,” Fundamental and Clinical Pharmacology,” Vol. 16, pp. 263-272.
Weier et al., 2003, “1,5-Diaryl substituted pyrazoles as p38 kinase inhibitors,” U.S. Pat. No. 6,509,361, granted Jan. 21, 2003.
Weier, R M, et al., 1999, “1,5-Diaryl Substituted Pyrazoles as P38 Kinase Inhibitors,” International (PCT) Patent Application publication no. WO 99/58523, published 18 Nov. 1999.
Wiley, J L, et al., 2001, “Novel Pyrazole Cannabinoids: Insights into CB1 Receptor Recognition and Activation,” J. Pharmacology and Experimental Therapeutics, Vol. 296, No. 3, pp. 1013-1022.
Wiley, J L, et al., 2002, “Resorcinol Derivatives: A Novel Template for the Development of Cannabinoid CB1/CB2 and CB2-Selective Agonists,” Journal of Pharmacology and Experimental Therapeutics, Vol. 301, No. 2, pp. 679-689.
Will R, Palmer R, Bhalla A K, Ring F, and Calin A, 1989, “Osteoporosis in early ankylosing spondylitis: a primary pathological event?,” Lancet, Vol. 2, Nos. 8678-8679, pp. 1483-1485.
Xiang, J. N., et al., 1998, “Novel Cannabinoid Receptor Modulators,” international (PCT) patent publication number WO 98/31227 published 23 Jul. 1998.
Yasuda, H, Shima, N, Nakagawa, N, Mochizuki, S I, Yano, K, Fujise, N, et al, 1998, “Identity of osteoclastogenesis inhibitory factor (OCIF) and osteoprotegerin (OPG): a mechanism by which OPG/OCIF inhibits osteoclastogenesis in vitro,” Endocrinology, Vol. 139, pp. 1329-1337.
Zygmunt P M, Petersson J, Andersson D A, Chuang H, Sorgard M, Di M, V, Julius D, Hogestatt E D, 1999, “Vanilloid receptors on sensory nerves mediate the vasodilator action of anandamide,” Nature, Vol. 400, pp. 452-457.

1. Use of a cannabinoid receptor inverse agonist or a cannabinoid receptor neutral antagonist for the manufacture of a medicament for the treatment of a bone disorder.

2-108. (canceled)
